(19) United States
(12) Patent Application Publication
(10) Pub. No.: US 2007/0128639 A1
(43) Pub. Date: Jun. 7, 2007

(54) Molecular profiling of cancer

(75) Inventors: Arul M. Chinnaiyan, Plymouth, MI (US); Sooryanarayana Lnu, Ann Arbor, MI (US)

Correspondence Address: MEDLEN & CARROLL, LLP, 101 HOWARD STREET, SUITE 350, SAN FRANCISCO, CA, 94105, US

(73) Assignee: Regents of the University of Michigan, Ann Arbor, MI (US)

(21) Appl. No.: 11/592,087

(22) Filed: Nov. 2, 2006

Related U.S. Application Data
(60) Provisional application No. 60/732,859, filed on Nov. 2, 2005.

(51) Int. Cl. C12Q 1/68; G01N 33/574

(52) U.S. Cl. 435/6; 435/7.23

(57) ABSTRACT

The present invention relates to compositions and methods for cancer diagnostics, including but not limited to, cancer markers. In particular, the present invention provides cancer markers useful in the diagnosis and characterization of prostate and breast cancers.

This application claims priority to provisional patent application Ser. No. 60/732,859, filed Nov. 2, 2005 which is herein incorporated by reference in its entirety.

This invention was made with government support under grant numbers P50CA69568, R01AG21404 and UO1 CA111275-01 awarded by the National Institutes of Health. The government has certain rights in the invention.

### FIELD OF THE INVENTION

The present invention relates to compositions and methods for cancer diagnostics, including but not limited to, cancer markers. In particular, the present invention provides cancer markers useful in the diagnosis and characterization of prostate and breast cancers.

### BACKGROUND OF THE INVENTION

Afflicting one out of nine men over age 65, prostate cancer (PCA) is a leading cause of male cancer-related death, second only to lung cancer (Abate-Shen and Shen, Genes Dev 14:2410 [2000]; Ruijter et al., Endocr Rev, 20:22 [1999]). The American Cancer Society estimates that about 184,500 American men will be diagnosed with prostate cancer and 39,200 will die in 2001.

Prostate cancer is typically diagnosed with a digital rectal exam and/or prostate specific antigen (PSA) screening. An elevated serum PSA level can indicate the presence of PCA. PSA is used as a marker for prostate cancer because it is secreted only by prostate cells. A healthy prostate will produce a stable amount—typically below 4 nanograms per milliliter, or a PSA reading of “4” or less—whereas cancer cells produce escalating amounts that correspond with the severity of the cancer. A level between 4 and 10 may raise a doctor's suspicion that a patient has prostate cancer, while amounts above 50 may show that the tumor has spread elsewhere in the body.

When PSA or digital tests indicate a strong likelihood that cancer is present, a transrectal ultrasound (TRUS) is used to map the prostate and show any suspicious areas. Biopsies of various sectors of the prostate are used to determine if prostate cancer is present. Treatment options depend on the stage of the cancer. Men with a 10-year life expectancy or less who have a low Gleason number and whose tumor has not spread beyond the prostate are often treated with watchful waiting (no treatment). Treatment options for more aggressive cancers include surgical treatments such as radical prostatectomy (RP), in which the prostate is completely removed (with or without nerve sparing techniques) and radiation, applied through an external beam that directs the dose to the prostate from outside the body or via low-dose radioactive seeds that are implanted within the prostate to kill cancer cells locally. Anti-androgen hormone therapy is also used, alone or in conjunction with surgery or radiation. Hormone therapy uses luteinizing hormone-releasing hormones (LH-RH) analogs, which block the pituitary from producing hormones that stimulate testosterone production. Patients must have injections of LH-RH analogs for the rest of their lives.

While surgical and hormonal treatments are often effective for localized PCA, advanced disease remains essentially incurable. Androgen ablation is the most common therapy for advanced PCA, leading to massive apoptosis of androgen-dependent malignant cells and temporary tumor regression. In most cases, however, the tumor reemerges with a vengeance and can proliferate independent of androgen signals.

The advent of prostate specific antigen (PSA) screening has led to earlier detection of PCA and significantly reduced PCA-associated fatalities. However, the impact of PSA screening on cancer-specific mortality is still unknown pending the results of prospective randomized screening studies (Etzioni et al., J. Natl. Cancer Inst., 91:1033 [1999]; Maattanen et al., Br. J. Cancer 79:1210 [1999]; Schroder et al., J. Natl. Cancer Inst., 90:1817 [1998]). A major limitation of the serum PSA test is a lack of prostate cancer sensitivity and specificity especially in the intermediate range of PSA detection (4-10 ng/ml). Elevated serum PSA levels are often detected in patients with non-malignant conditions such as benign prostatic hyperplasia (BPH) and prostatitis, and provide little information about the aggressiveness of the cancer detected. Coincident with increased serum PSA testing, there has been a dramatic increase in the number of prostate needle biopsies performed (Jacobsen et al., JAMA 274:1445 [1995]). This has resulted in a surge of equivocal prostate needle biopsies (Epstein and Potter J. Urol., 166:402 [2001]). Thus, development of additional serum and tissue biomarkers to supplement PSA screening is needed.

### SUMMARY OF THE INVENTION

The present invention relates to compositions and methods for cancer diagnostics, including but not limited to, cancer markers. In particular, the present invention provides cancer markers useful in the diagnosis and characterization of prostate and breast cancers.

For Example, in some embodiments, the present invention provides a method for characterizing prostate tissue in a subject, comprising: providing a prostate tissue sample from a subject; and detecting the level of expression of a cancer marker (e.g., E2 ubiquitin ligase, UBc9, the cytosolic phosphoprotein stathmin, the death receptor DR3, and the Aurora A kinase (STK15), KRIP1 (KAP-1), Dynamin, CDK7, LAP2, Myosin VI, ICBP90, ILP/XIAP, CamKK, JAM1, PICIn, or p23) in the sample, thereby characterizing the prostate tissue sample. In some embodiments, the detecting the level of expression of a cancer marker comprises detecting the presence of cancer marker mRNA (e.g., by exposing the cancer marker MRNA to a nucleic acid probe complementary to the cancer marker MRNA). In other embodiments, detecting the level of expression of a cancer marker comprises detecting the presence of a cancer marker polypeptide (e.g., by exposing the cancer marker polypeptide to an antibody specific to the cancer marker polypeptide and detecting the binding of the antibody to the cancer marker polypeptide). In some embodiments, the subject is a human subject. In some embodiments, the sample comprises tumor tissue. In some embodiments, characterizing the prostate tissue comprises identifying a stage of prostate cancer in the prostate tissue (e.g., prostate carcinoma or metastatic prostate carcinoma). In some embodiments, the method further comprises the step providing a prognosis to the subject (e.g., the risk of developing prostate cancer).

The present invention further provides a kit for characterizing prostate tissue in a subject, comprising: a reagent capable of (e.g., sufficient to) specifically detect the level of expression of a cancer marker (e.g., E2 ubiquitin ligase, UBc9, the cytosolic phosphoprotein stathmin, the death receptor DR3, and the Aurora A kinase (STK15), KRIP1 (KAP-1), Dynamin, CDK7, LAP2, Myosin VI, ICBP90, ILP/XIAP, CamKK, JAM1, PICIn, or p23); and optionally, instructions for using the kit for characterizing prostate tissue in the subject. In some embodiments, the reagent comprises a nucleic acid probe complementary to the cancer marker mRNA. In other embodiments, the reagent comprises an antibody that specifically binds to the cancer marker polypeptide. In some embodiments, the instructions comprise instructions required by the United States Food and Drug Administration for use in in vitro diagnostic products. In some embodiments, the kit comprises software that assists in the collection of, analysis of, interpretation of, and/or display of data or results generated by or from the reagents.

In still further embodiments, the present invention provides a method for characterizing breast tissue in a subject, comprising: providing a breast tissue sample from a subject; and detecting the level of expression of a cancer maker (e.g., CamKK, Myosin VI, Auroara A, exportin, BM28, CDK7, TIP60, or p16 INK 4a) in the sample, thereby characterizing the breast tissue sample. In some embodiments, the detecting the level of expression of a cancer marker comprises detecting the presence of cancer marker mRNA (e.g., by exposing the cancer marker mRNA to a nucleic acid probe complementary to the cancer marker mRNA). In other embodiments, detecting the level of expression of a cancer marker comprises detecting the presence of a cancer marker polypeptide (e.g., by exposing the cancer marker polypeptide to an antibody specific to the cancer marker polypeptide and detecting the binding of the antibody to the cancer marker polypeptide). In some embodiments, the subject is a human subject. In some embodiments, the sample comprises tumor tissue. In some embodiments, the method further comprises the step of providing a prognosis to the subject (e.g., the risk of developing breast cancer).

In yet other embodiments, the present invention provides a kit for characterizing breast tissue in a subject, comprising: a reagent capable of (e.g., sufficient to) specifically detect the level of expression of a cancer marker (e.g., CamKK, Myosin VI, Auroara A, exportin, BM28, CDK7, TIP60, or p16 INK 4a); and optionally, instructions for using the kit for characterizing breast tissue in the subject. In some embodiments, the reagent comprises a nucleic acid probe complementary to the cancer marker mRNA. In other embodiments, the reagent comprises an antibody that specifically binds to the cancer marker polypeptide. In some embodiments, the instructions comprise instructions required by the United States Food and Drug Administration for use in in vitro diagnostic products. In some embodiments, the kit comprises software that assists in the collection of, analysis of, interpretation of, and/or display of data or results generated by or from the reagents.

### DESCRIPTION OF THE FIGURES

FIG. 1 shows high-throughput immunoblot analysis to define proteomic alterations in prostate cancer progression. A, A flowchart of the general methodology employed to profile proteomic alterations in tissue extracts. B, Representative high-throughput immunoblots performed for pooled benign, clinically localized prostate cancer and metastatic prostate cancer tissues.

FIG. 2 shows tissue microarray analyses of protein markers deregulated in prostate cancer progression. A. Selected images of tissue microarray elements representing immunohistochemical analysis of proteins altered in prostate cancer progression. B, Cluster analysis of twenty proteins dysregulated in prostate cancer progression evaluated for in situ protein levels by tissue microarrays.

FIG. 3 shows integrative proteomic and transcriptomic analysis of prostate cancer progression. A, Color map of integrative analysis relating protein alterations to gene expression in clinically localized prostate cancer relative to benign prostate tissue. B, As in A except the integrative analysis was carried out between metastatic prostate cancer relative to clinically localized prostate cancer. C, Conventional immunoblot validation of selected proteins differentially expressed between metastatic prostate cancer and clinically localized prostate cancer.

FIG. 4 shows proteomic alterations in metastatic prostate cancer nominate gene predictors of cancer aggressiveness. A, A concordant 44-gene predictor was developed based on proteomic alterations that were concordant with gene expression (FIG. 3B) and subsequently evaluated for prognostic utility. B, The concordant 44-gene predictor and the refined concordant 9-gene predictor were evaluated in an independent prostate cancer profiling dataset. C, Same as A, except the concordant 44-gene predictor was evaluated in other solid tumors.

FIG. 5 shows integrative molecular analysis of cancer to identify gene predictors of clinical outcome.

FIG. 6 shows integrative genomic and proteomic analysis of pooled and individual prostate tissue extracts. FIG. 6A shows color maps of integrative analyses relating protein alterations observed in pooled tissues by immunoblotting and transcript alterations observed in the pooled and individual tissues by gene expression analyses. FIG. 6B shows color maps depicting integrative genomic and proteomic analysis of individual prostate tissue samples.

FIG. 7 shows validation of proteomic alterations in prostate cancer by conventional immunoblot analysis.

FIG. 8 shows high-resolution images from FIG. 2. FIG. 8A shows high resolution images of the staining shown in FIG. 2. FIG. 8B represents the cluster analysis of twenty proteins dysregulated in prostate cancer progression evaluated for in situ protein levels by tissue microarrays.

FIG. 9 shows high-resolution matrix maps described in FIG. 3A. A, Color map of integrative analysis relating protein alterations to gene expression in clinically localized prostate cancer relative to benign prostate tissue. B, As in A except the integrative analysis was carried out between metastatic prostate cancer relative to clinically localized prostate cancer.

FIG. 10 shows high-resolution matrix maps for proteomic alterations in metastatic prostate cancer nominate gene predictors of prostate cancer aggressiveness. A, A concordant 44-gene predictor was developed based on proteomic alterations that were concordant with gene expression (FIG. 3B) and subsequently evaluated for prognostic utility. B, The concordant 44-gene predictor was evaluated in an independent prostate cancer profiling dataset. C. Same as A, except the refined concordant 9-gene predictor was evaluated in the Yu et al. study. D. Same as B, except the refined concordant 9-gene predictor was evaluated by using the Glinsky et al. study as a validation dataset.

FIG. 11 shows high-resolution matrix maps described in FIG. 4C with the addition of the Van't Veer breast cancer profiling dataset. A, A concordant 44-gene predictor was developed based on proteomic alterations that were concordant with gene expression (FIG. 3B). B, The concordant 44-gene predictor was evaluated in an independent prostate cancer profiling dataset. C. Same as A, except the refined concordant 9-gene predictor was evaluated. D. Same as B, except the refined concordant 9-gene predictor was evaluated by using the Glinsky et al. study as a validation dataset.

FIG. 12 shows High-resolution matrix maps described in FIG. 5C with the addition of the Van't Veer breast cancer profiling dataset.

FIG. 13 shows an immunoblot of breast cancer markers.

FIG. 14 shows Table 9.

FIG. 15 shows Table 10.

### GENERAL DESCRIPTION

Multiple molecular alterations occur during cancer development. To begin to understand these processes with a systems perspective, there is a need to characterize and integrate these components. Experiments conducted during the course of development of the present invention integrated such disparate molecular data as RNA expression profiling and protein expression in prostate and breast cancer.

A high-throughput immunoblot approach was used to characterize proteomic alterations in human prostate cancer progression focusing on the transition of clinically localized disease to metastatic disease. This approach revealed over one hundred proteomic alterations in prostate cancer progression. Furthermore, these proteomic profiles were integrated with MRNA transcript data from independent expression profiling datasets. Proteins that were qualitatively concordant with gene expression could be used as a predictor of clinical outcome. In other words, this integrative approach revealed the presence of an “aggressive signature” in clinically localized prostate tumors.

Prostate cancer is a highly prevalent disease in older men of the Western world (Chan et al., J Urol 172, S13-16, 2004; Linton and Hamdy, Cancer Treat Rev 29, 151-160, 2003). Unlike other cancers, more men die with prostate cancer than from the disease (Albertsen et al., Jama 280, 975-980, 1998; Johansson et al., Jama 277, 467-471, 1997). Deciphering the molecular networks that distinguish progressive disease from nonprogressive disease sheds light into the biology of aggressive prostate cancer as well as leads to the identification of biomarkers that aid in the selection of patients that should be treated (Kumar-Sinha and Chinnaiyan, Urology 62 Suppl 1, 19-35, 2003). To begin to understand prostate cancer progression with a systems perspective, it is helpful to characterize and integrate the molecular components involved (Grubb et al., Proteomics 3, 2142-21462003; Hood et al., Science 306, 640-643, 2004; Paweletz et al., Oncogene 20, 1981-1989, 2001; Petricoin et al., J Natl Cancer Inst 94, 1576-1578, 2002). A number of groups have employed gene expression microarrays to profile prostate cancer tissues (Dhanasekaran et al., Nature 412, 822-826, 2001; Lapointe et al., Proc Natl Acad Sci USA 101, 811-816, 2004; LaTulippe et al., Cancer Res 62, 4499-4506, 2002; Luo et al., Cancer Res 61, 4683-4688, 2001; Luo et al., Mol Carcinog 33, 25-35, 2002b; Magee et al., Cancer Res 61, 5692-5696, 2001; Singh et al., Cancer Cell 1, 203-209, 2002; Welsh et al., Cancer Res 61, 5974-5978, 2001; Yu et al., J Clin Oncol 22, 2790-2799, 2004) as well as other tumors (Alizadeh et al., Nature 403, 503-511, 2000; Golub et al., Science 286, 531-537, 1999; Hedenfalk et al., N Engl J Med 344, 539-548, 2001; Perou et al., Nature 406, 747-752, 2000) at the transcriptome level but much less work has been done at the protein level. Proteins, as opposed to nucleic acids, represent the functional effectors of cancer progression and thus serve as therapeutic targets as well as markers of disease.

In experiments conducted during the course of development of the present invention, a high-throughput immunoblot approach was utilized to characterize proteomic alterations in human prostate cancer progression focusing on the transition from clinically localized prostate cancer to metastatic disease. Using an integrative approach, proteomic profiles with mRNA transcript data from several experiments were analyzed. The analyses also indicated that the proteins that were qualitatively concordant with gene expression could be used to define a multiplex gene predictor of clinical outcome.

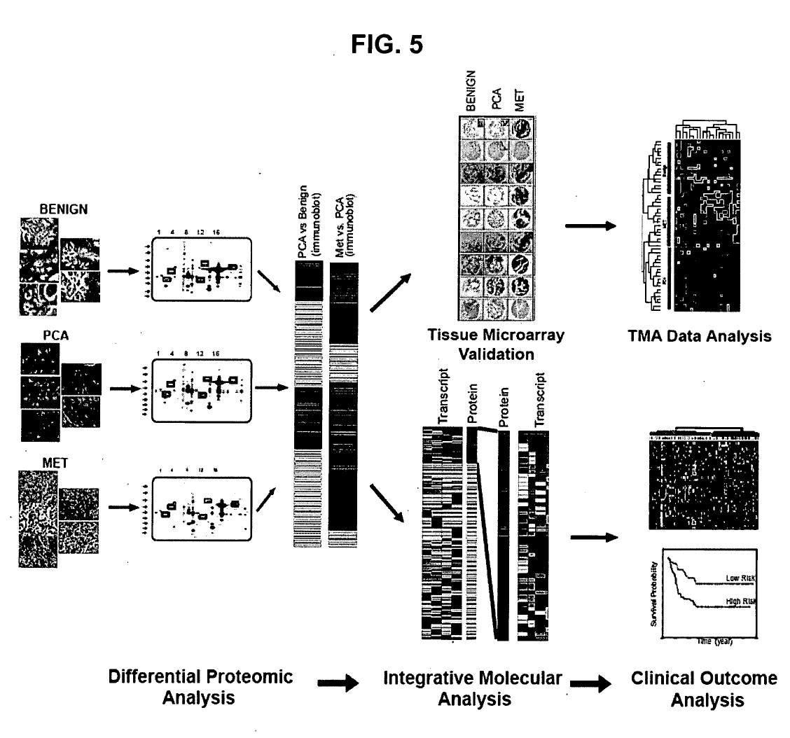

The present invention provides a general framework for the integrative analysis of protein and transcriptomic data from human tumors (FIG. 5). Proteomic profiling of prostate cancer progression identified over one hundred altered proteins in the transition from clinically localized to metastatic disease (a significant fraction of which were androgen regulated). While this approach was useful to integrate high-throughput immunoblot data, the general paradigm can also be applied to mass spectrometry or protein microarray based technologies. Differential proteins were then mapped to mRNA transcript levels to assess mRNA/protein concordance levels in a human disease state. Gene expression alterations that matched protein alterations qualitatively could be used as predictors of prostate cancer progression in clinically confined disease. Together, this shows that clinically aggressive prostate cancer bears a “signature” set of genes/proteins that is characteristic of metastatic disease. The observation that the concordant proteomic/genomic signature can be applied to other solid tumors shows commonalities in the undifferentiated state of advanced tumors.

### DEFINITIONS

To facilitate an understanding of the present invention, a number of terms and phrases are defined below:

The term “epitope” as used herein refers to that portion of an antigen that makes contact with a particular antibody.

When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

As used herein, the term “siRNAs” refers to small interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to, or substantially complementary to, a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “subject suspected of having cancer” refers to a subject that presents one or more symptoms indicative of a cancer (e.g., a noticeable lump or mass) or is being screened for a cancer (e.g., during a routine physical). A subject suspected of having cancer may also have one or more risk factors. A subject suspected of having cancer has generally not been tested for cancer. However, a “subject suspected of having cancer” encompasses an individual who has received an initial diagnosis (e.g., a CT scan showing a mass or increased PSA level) but for whom the stage of cancer is not known. The term further includes people who once had cancer (e.g., an individual in remission).

As used herein, the term “subject at risk for cancer” refers to a subject with one or more risk factors for developing a specific cancer. Risk factors include, but are not limited to, gender, age, genetic predisposition, environmental expose, previous incidents of cancer, preexisting non-cancer diseases, and lifestyle.

As used herein, the term “characterizing cancer in subject” refers to the identification of one or more properties of a cancer sample in a subject, including but not limited to, the presence of benign, pre-cancerous or cancerous tissue, the stage of the cancer, and the subject's prognosis. Cancers may be characterized by the identification of the expression of one or more cancer marker genes, including but not limited to, the cancer markers disclosed herein.

As used herein, the term “characterizing prostate tissue in a subject” refers to the identification of one or more properties of a prostate tissue sample (e.g., including but not limited to, the presence of cancerous tissue, the presence of pre-cancerous tissue that is likely to become cancerous, and the presence of cancerous tissue that is likely to metastasize). In some embodiments, tissues are characterized by the identification of the expression of one or more cancer marker genes, including but not limited to, the cancer markers disclosed herein.

As used herein, the term “cancer marker genes” refers to a gene whose expression level, alone or in combination with other genes, is correlated with cancer or prognosis of cancer. The correlation may relate to either an increased or decreased expression of the gene. For example, the expression of the gene may be indicative of cancer, or lack of expression of the gene may be correlated with poor prognosis in a cancer patient. Cancer marker expression may be characterized using any suitable method, including but not limited to, those described in the illustrative Examples below.

As used herein, the term “a reagent that specifically detects expression levels” refers to reagents used to detect the expression of one or more genes (e.g., including but not limited to, the cancer markers of the present invention). Examples of suitable reagents include but are not limited to, nucleic acid probes capable of specifically hybridizing to the gene of interest, PCR primers capable of specifically amplifying the gene of interest, and antibodies capable of specifically binding to proteins expressed by the gene of interest. Other non-limiting examples can be found in the description and examples below.

As used herein, the term “detecting a decreased or increased expression relative to non-cancerous prostate control” refers to measuring the level of expression of a gene (e.g., the level of mRNA or protein) relative to the level in a non-cancerous prostate control sample. Gene expression can be measured using any suitable method, including but not limited to, those described herein.

As used herein, the term “detecting a change in gene expression (e.g., cancer marker gene expression) in said prostate cell sample in the presence of said test compound relative to the absence of said test compound” refers to measuring an altered level of expression (e.g., increased or decreased) in the presence of a test compound relative to the absence of the test compound. Gene expression can be measured using any suitable method, including but not limited to, those described in the Examples below.

As used herein, the term “instructions for using said kit for detecting cancer in said subject” includes instructions for using the reagents contained in the kit for the detection and characterization of cancer in a sample from a subject. In some embodiments, the instructions further comprise the statement of intended use required by the U.S. Food and Drug Administration (FDA) in labeling in vitro diagnostic products.

As used herein, the term “prostate cancer expression profile map” refers to a presentation of expression levels of genes in a particular type of prostate tissue (e.g., primary, metastatic, and pre-cancerous prostate tissues). The map may be presented as a graphical representation (e.g., on paper or on a computer screen), a physical representation (e.g., a gel or array) or a digital representation stored in computer memory. Each map corresponds to a particular type of prostate tissue (e.g., primary, metastatic, and pre-cancerous) and thus provides a template for comparison to a patient sample. In preferred embodiments, maps are generated from pooled samples comprising tissue samples from a plurality of patients with the same type of tissue.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refer to a device that is able to read a program from a computer memory (e.g., ROM or other computer memory) and perform a set of steps according to the program.

As used herein, the term “stage of cancer” refers to a qualitative or quantitative assessment of the level of advancement of a cancer. Criteria used to determine the stage of a cancer include, but are not limited to, the size of the tumor, whether the tumor has spread to other parts of the body and where the cancer has spread (e.g., within the same organ or region of the body or to another organ).

As used herein, the term “providing a prognosis” refers to providing information regarding the impact of the presence of cancer (e.g., as determined by the diagnostic methods of the present invention) on a subject's future health (e.g., expected morbidity or mortality, the likelihood of getting cancer, and the risk of metastasis).

As used herein, the term “initial diagnosis” refers to results of initial cancer diagnosis (e.g. the presence or absence of cancerous cells). An initial diagnosis does not include information about the stage of the cancer of the risk of metastasis.

As used herein, the term “biopsy tissue” refers to a sample of tissue (e.g., prostate tissue) that is removed from a subject for the purpose of determining if the sample contains cancerous tissue. In some embodiment, biopsy tissue is obtained because a subject is suspected of having cancer. The biopsy tissue is then examined (e.g., by microscopy) for the presence or absence of cancer.

As used herein, the term “non-human animals” refers to all non-human animals including, but are not limited to, vertebrates such as rodents, non-human primates, ovines, bovines, ruminants, lagomorphs, porcines, caprines, equines, canines, felines, aves, etc.

As used herein, the term “gene transfer system” refers to any means of delivering a composition comprising a nucleic acid sequence to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system”refers to gene transfer systems comprising intact or altered viruses belonging to the family Adenoviridae.

As used herein, the term “site-specific recombination target sequences” refers to nucleic acid sequences that provide recognition sequences for recombination factors and the location where recombination takes place.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1 -methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region may be present in a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under ‘medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄ H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄ H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄ H₂O and 1.85 g/l EDTA, pH adjusted to 7.4 with NaOH), 0.1% SDS, 5× Denhardt's reagent [50×Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formanide in the hybridization solution, etc.) (see definition above for “stringency”).

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to at least a portion of another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (10 nucleotides, 20, 30, 40, 50, 100, 200, etc.).

As used herein, the term “amplification reagents” refers to those reagents (deoxyribonucleotide triphosphates, buffer, etc.), needed for amplification except for primers, nucleic acid template and the amplification enzyme. Typically, amplification reagents along with other reaction components are placed and contained in a reaction vessel (test tube, microwell, etc.).

As used herein, the terms “restriction endonucleases” and “restriction enzymes” refer to bacterial enzymes, each of which cut double-stranded DNA at or near a specific nucleotide sequence.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence minus one amino acid.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al., Molecular Cloning: A Laboratory Manual, Cold Spring Harbor Press, New York, pp 9.31-9.58 [1989]).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, et al., supra, pp 7.39-7.52 [1989]).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabeled antibodies.

The term “transgene” as used herein refers to a foreign gene that is placed into an organism by, for example, introducing the foreign gene into newly fertilized eggs or early embryos. The term “foreign gene” refers to any nucleic acid (e.g., gene sequence) that is introduced into the genome of an animal by experimental manipulations and may include gene sequences found in that animal so long as the introduced gene does not reside in the same location as does the naturally occurring gene.

As used herein, the term “vector” is used in reference to nucleic acid molecules that transfer DNA segment(s) from one cell to another. The term “vehicle” is sometimes used interchangeably with “vector.” Vectors are often derived from plasmids, bacteriophages, or plant or animal viruses.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “overexpression” and “overexpressing” and grammatical equivalents, are used in reference to levels of mRNA to indicate a level of expression approximately 3-fold higher (or greater) than that observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

As used herein, the term “selectable marker” refers to the use of a gene that encodes an enzymatic activity that confers the ability to grow in medium lacking what would otherwise be an essential nutrient (e.g. the HIS3 gene in yeast cells); in addition, a selectable marker may confer resistance to an antibiotic or drug upon the cell in which the selectable marker is expressed. Selectable markers may be “dominant”; a dominant selectable marker encodes an enzymatic activity that can be detected in any eukaryotic cell line. Examples of dominant selectable markers include the bacterial aminoglycoside 3′ phosphotransferase gene (also referred to as the neo gene) that confers resistance to the drug G418 in mammalian cells, the bacterial hygromycin G phosphotransferase (hyg) gene that confers resistance to the antibiotic hygromycin and the bacterial xanthine-guanine phosphoribosyl transferase gene (also referred to as the gpt gene) that confers the ability to grow in the presence of mycophenolic acid. Other selectable markers are not dominant in that their use must be in conjunction with a cell line that lacks the relevant enzyme activity. Examples of non-dominant selectable markers include the thymidine kinase (tk) gene that is used in conjunction with tk⁻ cell lines, the CAD gene that is used in conjunction with CAD-deficient cells and the mammalian hypoxanthine-guanine phosphoribosyl transferase (hprt) gene that is used in conjunction with hprt⁻ cell lines. A review of the use of selectable markers in mammalian cell lines is provided in Sambrook, J. et al., Molecular Cloning: A Laboratory Manual, 2nd ed., Cold Spring Harbor Laboratory Press, New York (1989) pp. 16.9-16.15.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using the screening methods of the present invention. In some embodiments of the present invention, test compounds include antisense compounds.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

### DETAILED DESCRIPTION OF THE INVENTION

The present invention relates to compositions and methods for cancer diagnostics, including but not limited to, cancer markers. In particular, the present invention provides cancer markers and cancer marker profiles associated with prostate and breast cancers. Accordingly, the present invention provides method of characterizing prostate and breast tissues, kits for the detection of markers, as well as drug screening and therapeutic applications.

I. Cancer Markers

The present invention provides markers whose expression is specifically altered in cancerous prostate and breast tissues. Such markers find use in the diagnosis and characterization of prostate and breast cancer.

A. Identification of Markers

Experiments conducted during the course of development of the present invention identified markers with altered expression levels in prostate cancer relative to normal prostate or in metastatic prostate cancer relative to local prostate cancer. Exemplary markers are described in the Figure and Tables herein. In some preferred embodiments, prostate cancer markers include, but are not limited to, E2 ubiquitin ligase, UBc9, the cytosolic phosphoprotein stathmin, the death receptor DR3, the Aurora A kinase (STK15), KRIP1(KAP-1), Dynamin, CDK7, LAP2, Myosin VI, ICBP90, ILP/XIAP, CamKK, JAM1, PICIn, or p23.

Further experiments conducted during the course of development of the present invention identified breast cancer markers. Exemplary markers include, but are not limited to, CamKK, Myosin VI, Auroara A, exportin, BM28, CDK7, TIP60, or 16 INK 4a.

B. Detection of Markers

In some embodiments, the present invention provides methods for detection of expression of cancer markers (e.g., prostate or breast cancer markers). In preferred embodiments, expression is measured directly (e.g., at the RNA or protein level). In some embodiments, expression is detected in tissue samples (e.g., biopsy tissue). In other embodiments, expression is detected in bodily fluids (e.g., including but not limited to, plasma, serum, whole blood, mucus, and urine). The present invention further provides panels and kits for the detection of markers. In preferred embodiments, the presence of a cancer marker is used to provide a prognosis to a subject. The information provided is also used to direct the course of treatment. For example, if a subject is found to have a marker indicative of a highly metastasizing tumor, additional therapies (e.g., hormonal or radiation therapies) can be started at a earlier point when they are more likely to be effective (e.g., before metastasis). In addition, if a subject is found to have a tumor that is not responsive to hormonal therapy, the expense and inconvenience of such therapies can be avoided.

The present invention is not limited to the markers described above. Any suitable marker that correlates with cancer or the progression of cancer may be utilized, including but not limited to, those described in the illustrative examples below. Additional markers are also contemplated to be within the scope of the present invention. Any suitable method may be utilized to identify and characterize cancer markers suitable for use in the methods of the present invention, including but not limited to, those described in illustrative Examples below. For example, in some embodiments, markers identified as being up or down-regulated in PCA using the gene expression microarray methods of the present invention are further characterized using tissue microarray, immunohistochemistry, Northern blot analysis, siRNA or antisense RNA inhibition, mutation analysis, investigation of expression with clinical outcome, as well as other methods disclosed herein.

In some embodiments, the present invention provides a panel for the analysis of a plurality of markers. The panel allows for the simultaneous analysis of multiple markers correlating with carcinogenesis and/or metastasis. For example, a panel may include markers identified as correlating with cancerous tissue, metastatic cancer, localized cancer that is likely to metastasize, pre-cancerous tissue that is likely to become cancerous, and pre-cancerous tissue that is not likely to become cancerous. Depending on the subject, panels may be analyzed alone or in combination in order to provide the best possible diagnosis and prognosis. Markers for inclusion on a panel are selected by screening for their predictive value using any suitable method, including but not limited to, those described in the illustrative examples below.

In other embodiments, the present invention provides an expression profile map comprising expression profiles of cancers of various stages or prognoses (e.g., likelihood of future metastasis). Such maps can be used for comparison with patient samples. Any suitable method may be utilized, including but not limited to, by computer comparison of digitized data. The comparison data is used to provide diagnoses and/or prognoses to patients.

1. Detection of RNA

In some preferred embodiments, detection of prostate or breast cancer markers (e.g., including but not limited to, those disclosed herein) is detected by measuring the expression of corresponding MRNA in a tissue sample (e.g., prostate tissue). mRNA expression may be measured by any suitable method, including but not limited to, those disclosed below.

In some embodiments, RNA is detection by Northern blot analysis. Northern blot analysis involves the separation of RNA and hybridization of a complementary labeled probe.

In still further embodiments, RNA (or corresponding cDNA) is detected by hybridization to a oligonucleotide probe). A variety of hybridization assays using a variety of technologies for hybridization and detection are available. For example, in some embodiments, TaqMan assay (PE Biosystems, Foster City, Calif.; See e.g., U.S. Pat. Nos. 5,962,233 and 5,538,848, each of which is herein incorporated by reference) is utilized. The assay is performed during a PCR reaction. The TaqMan assay exploits the 5′-3′ exonuclease activity of the AMPLITAQ GOLD DNA polymerase. A probe consisting of an oligonucleotide with a 5′-reporter dye (e.g., a fluorescent dye) and a 3′-quencher dye is included in the PCR reaction. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In yet other embodiments, reverse-transcriptase PCR (RT-PCR) is used to detect the expression of RNA. In RT-PCR, RNA is enzymatically converted to complementary DNA or “cDNA” using a reverse transcriptase enzyme. The cDNA is then used as a template for a PCR reaction. PCR products can be detected by any suitable method, including but not limited to, gel electrophoresis and staining with a DNA specific stain or hybridization to a labeled probe. In some embodiments, the quantitative reverse transcriptase PCR with standardized mixtures of competitive templates method described in U.S. Pat. Nos. 5,639,606, 5,643,765, and 5,876,978 (each of which is herein incorporated by reference) is utilized.

2. Detection of Protein

In other embodiments, gene expression of cancer markers is detected by measuring the expression of the corresponding protein or polypeptide. Protein expression may be detected by any suitable method. In some embodiments, proteins are detected by immunohistochemistry. In other embodiments, proteins are detected by their binding to an antibody raised against the protein. The generation of antibodies is described below.

Antibody binding is detected by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a prognosis based on the presence or absence of a series of proteins corresponding to cancer markers is utilized.

In other embodiments, the immunoassay described in U.S. Pat. Nos. 5,599,677 and 5,672,480; each of which is herein incorporated by reference.

3. Data Analysis

In some embodiments, a computer-based analysis program is used to translate the raw data generated by the detection assay (e.g., the presence, absence, or amount of a given marker or markers) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., likelihood of metastasis) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease.

4. Kits

In yet other embodiments, the present invention provides kits for the detection and characterization of cancer (e.g. prostate or breast cancer). In some embodiments, the kits contain antibodies specific for a cancer marker, in addition to detection reagents and buffers. In other embodiments, the kits contain reagents specific for the detection of mRNA or cDNA (e.g., oligonucleotide probes or primers). In preferred embodiments, the kits contain all of the components necessary to perform a detection assay, including all controls, directions for performing assays, and any necessary software for analysis and presentation of results.

5. In vivo Imaging

In some embodiments, in vivo imaging techniques are used to visualize the expression of cancer markers in an animal (e.g., a human or non-human mammal). For example, in some embodiments, cancer marker mRNA or protein is labeled using an labeled antibody specific for the cancer marker. A specifically bound and labeled antibody can be detected in an individual using an in vivo imaging method, including, but not limited to, radionuclide imaging, positron emission tomography, computerized axial tomography, X-ray or magnetic resonance imaging method, fluorescence detection, and chemiluminescent detection. Methods for generating antibodies to the cancer markers of the present invention are described below.

The in vivo imaging methods of the present invention are useful in the diagnosis of cancers that express the cancer markers of the present invention (e.g., prostate cancer). In vivo imaging is used to visualize the presence of a marker indicative of the cancer. Such techniques allow for diagnosis without the use of an unpleasant biopsy. The in vivo imaging methods of the present invention are also useful for providing prognoses to cancer patients. For example, the presence of a marker indicative of cancers likely to metastasize can be detected. The in vivo imaging methods of the present invention can further be used to detect metastatic cancers in other parts of the body.

In some embodiments, reagents (e.g., antibodies) specific for the cancer markers of the present invention are fluorescently labeled. The labeled antibodies are introduced into a subject (e.g., orally or parenterally). Fluorescently labeled antibodies are detected using any suitable method (e.g., using the apparatus described in U.S. Pat. No. 6,198,107, herein incorporated by reference).

In other embodiments, antibodies are radioactively labeled. The use of antibodies for in vivo diagnosis is well known in the art. Sumerdon et al., (Nucl. Med. Biol 17:247-254 [1990] have described an optimized antibody-chelator for the radioimmunoscintographic imaging of tumors using Indium-111 as the label. Griffin et al., (J Clin Onc 9:631-640 [1991]) have described the use of this agent in detecting tumors in patients suspected of having recurrent colorectal cancer. The use of similar agents with paramagnetic ions as labels for magnetic resonance imaging is known in the art (Lauffer, Magnetic Resonance in Medicine 22:339-342 [1991]). The label used will depend on the imaging modality chosen. Radioactive labels such as Indium-111, Technetium-99m, or Iodine-131 can be used for planar scans or single photon emission computed tomography (SPECT). Positron emitting labels such as Fluorine-19 can also be used for positron emission tomography (PET). For MRI, paramagnetic ions such as Gadolinium (III) or Manganese (II) can be used.

Radioactive metals with half-lives ranging from 1 hour to 3.5 days are available for conjugation to antibodies, such as scandium-47 (3.5 days) gallium-67 (2.8 days), gallium-68 (68 minutes), technetiium-99m (6 hours), and indium-111 (3.2 days), of which gallium-67, technetium-99m, and indium-111 are preferable for gamma camera imaging, gallium-68 is preferable for positron emission tomography.

A useful method of labeling antibodies with such radiometals is by means of a bifunctional chelating agent, such as diethylenetriaminepentaacetic acid (DTPA), as described, for example, by Khaw et al. (Science 209:295 [1980]) for In-111 and Tc-99m, and by Scheinberg et al. (Science 215:1511 [1982]). Other chelating agents may also be used, but the 1-(p-carboxymethoxybenzyl)EDTA and the carboxycarbonic anhydride of DTPA are advantageous because their use permits conjugation without affecting the antibody's immunoreactivity substantially.

Another method for coupling DPTA to proteins is by use of the cyclic anhydride of DTPA, as described by Hnatowich et al. (Int. J. Appl. Radiat. Isot. 33:327 [1982]) for labeling of albumin with In-111, but which can be adapted for labeling of antibodies. A suitable method of labeling antibodies with Tc-99m which does not use chelation with DPTA is the pretinning method of Crockford et al., (U.S. Pat. No. 4,323,546, herein incorporated by reference).

A preferred method of labeling immunoglobulins with Tc-99m is that described by Wong et al. (Int. J. Appl. Radiat. Isot., 29:251 [1978]) for plasma protein, and recently applied successfully by Wong et al. (J. Nucl. Med., 23:229 [1981]) for labeling antibodies.

In the case of the radiometals conjugated to the specific antibody, it is likewise desirable to introduce as high a proportion of the radiolabel as possible into the antibody molecule without destroying its immunospecificity. A further improvement may be achieved by effecting radiolabeling in the presence of the specific cancer marker of the present invention, to insure that the antigen binding site on the antibody will be protected. The antigen is separated after labeling.

In still further embodiments, in vivo biophotonic imaging (Xenogen, Almeda, Calif.) is utilized for in vivo imaging. This real-time in vivo imaging utilizes luciferase. The luciferase gene is incorporated into cells, microorganisms, and animals (e.g., as a fusion protein with a cancer marker of the present invention). When active, it leads to a reaction that emits light. A CCD camera and software is used to capture the image and analyze it.

II. Antibodies

The present invention provides isolated antibodies. In preferred embodiments, the present invention provides monoclonal antibodies that specifically bind to an isolated polypeptide comprised of at least five amino acid residues of the cancer markers described herein. These antibodies find use in the diagnostic methods described herein.

An antibody against a protein of the present invention may be any monoclonal or polyclonal antibody, as long as it can recognize the protein. Antibodies can be produced by using a protein of the present invention as the antigen according to a conventional antibody or antiserum preparation process.

The present invention contemplates the use of both monoclonal and polyclonal antibodies. Any suitable method may be used to generate the antibodies used in the methods and compositions of the present invention, including but not limited to, those disclosed herein. For example, for preparation of a monoclonal antibody, protein, as such, or together with a suitable carrier or diluent is administered to an animal (e.g., a mammal) under conditions that permit the production of antibodies. For enhancing the antibody production capability, complete or incomplete Freund's adjuvant may be administered. Normally, the protein is administered once every 2 weeks to 6 weeks, in total, about 2 times to about 10 times. Animals suitable for use in such methods include, but are not limited to, primates, rabbits, dogs, guinea pigs, mice, rats, sheep, goats, etc.

For preparing monoclonal antibody-producing cells, an individual animal whose antibody titer has been confirmed (e.g., a mouse) is selected, and 2 days to 5 days after the final immunization, its spleen or lymph node is harvested and antibody-producing cells contained therein are fused with myeloma cells to prepare the desired monoclonal antibody producer hybridoma. Measurement of the antibody titer in antiserum can be carried out, for example, by reacting the labeled protein, as described hereinafter and antiserum and then measuring the activity of the labeling agent bound to the antibody. The cell fusion can be carried out according to known methods, for example, the method described by Koehler and Milstein (Nature 256:495 [1975]). As a fusion promoter, for example, polyethylene glycol (PEG) or Sendai virus (HVJ), preferably PEG is used.

Examples of myeloma cells include NS-1, P3U1, SP2/0, AP-1 and the like. The proportion of the number of antibody producer cells (spleen cells) and the number of myeloma cells to be used is preferably about 1:1 to about 20:1. PEG (preferably PEG 1000-PEG 6000) is preferably added in concentration of about 10% to about 80%. Cell fusion can be carried out efficiently by incubating a mixture of both cells at about 20° C. to about 40° C., preferably about 30° C. to about 37° C. for about 1 minute to 10 minutes.

Various methods may be used for screening for a hybridoma producing the antibody (e.g., against a tumor antigen or autoantibody of the present invention). For example, where a supernatant of the hybridoma is added to a solid phase (e.g., microplate) to which antibody is adsorbed directly or together with a carrier and then an anti-immunoglobulin antibody (if mouse cells are used in cell fusion, anti-mouse immunoglobulin antibody is used) or Protein A labeled with a radioactive substance or an enzyme is added to detect the monoclonal antibody against the protein bound to the solid phase. Alternately, a supernatant of the hybridoma is added to a solid phase to which an anti-immunoglobulin antibody or Protein A is adsorbed and then the protein labeled with a radioactive substance or an enzyme is added to detect the monoclonal antibody against the protein bound to the solid phase.

Selection of the monoclonal antibody can be carried out according to any known method or its modification. Normally, a medium for animal cells to which HAT (hypoxanthine, aminopterin, thymidine) are added is employed. Any selection and growth medium can be employed as long as the hybridoma can grow. For example, RPMI 1640 medium containing 1% to 20%, preferably 10% to 20% fetal bovine serum, GIT medium containing 1% to 10% fetal bovine serum, a serum free medium for cultivation of a hybridoma (SFM-101, Nissui Seiyaku) and the like can be used. Normally, the cultivation is carried out at 20° C. to 40° C., preferably 37° C. for about 5 days to 3 weeks, preferably 1 week to 2 weeks under about 5% CO₂ gas. The antibody titer of the supernatant of a hybridoma culture can be measured according to the same manner as described above with respect to the antibody titer of the anti-protein in the antiserum.

Separation and purification of a monoclonal antibody (e.g., against a cancer marker of the present invention) can be carried out according to the same manner as those of conventional polyclonal antibodies such as separation and purification of immunoglobulins, for example, salting-out, alcoholic precipitation, isoelectric point precipitation, electrophoresis, adsorption and desorption with ion exchangers (e.g., DEAE), ultracentrifugation, gel filtration, or a specific purification method wherein only an antibody is collected with an active adsorbent such as an antigen-binding solid phase, Protein A or Protein G and dissociating the binding to obtain the antibody.

Polyclonal antibodies may be prepared by any known method or modifications of these methods including obtaining antibodies from patients. For example, a complex of an immunogen (an antigen against the protein) and a carrier protein is prepared and an animal is immunized by the complex according to the same manner as that described with respect to the above monoclonal antibody preparation. A material containing the antibody against is recovered from the immunized animal and the antibody is separated and purified.

As to the complex of the immunogen and the carrier protein to be used for immunization of an animal, any carrier protein and any mixing proportion of the carrier and a hapten can be employed as long as an antibody against the hapten, which is crosslinked on the carrier and used for immunization, is produced efficiently. For example, bovine serum albumin, bovine cycloglobulin, keyhole limpet hemocyanin, etc. may be coupled to an hapten in a weight ratio of about 0.1 part to about 20 parts, preferably, about 1 part to about 5 parts per 1 part of the hapten.

In addition, various condensing agents can be used for coupling of a hapten and a carrier. For example, glutaraldehyde, carbodiimide, maleimide activated ester, activated ester reagents containing thiol group or dithiopyridyl group, and the like find use with the present invention. The condensation product as such or together with a suitable carrier or diluent is administered to a site of an animal that permits the antibody production. For enhancing the antibody production capability, complete or incomplete Freund's adjuvant may be administered. Normally, the protein is administered once every 2 weeks to 6 weeks, in total, about 3 times to about 10 times.

The polyclonal antibody is recovered from blood, ascites and the like, of an animal immunized by the above method. The antibody titer in the antiserum can be measured according to the same manner as that described above with respect to the supernatant of the hybridoma culture. Separation and purification of the antibody can be carried out according to the same separation and purification method of immunoglobulin as that described with respect to the above monoclonal antibody.

The protein used herein as the immunogen is not limited to any particular type of immunogen. For example, a cancer marker of the.present invention (further including a gene having a nucleotide sequence partly altered) can be used as the immunogen. Further, fragments of the protein may be used. Fragments may be obtained by any methods including, but not limited to expressing a fragment of the gene, enzymatic processing of the protein, chemical synthesis, and the like.

III. Drug Screening

In some embodiments, the present invention provides drug screening assays (e.g., to screen for anticancer drugs). The screening methods of the present invention utilize cancer markers identified using the methods of the present invention. For example, in some embodiments, the present invention provides methods of screening for compound that alter (e.g., increase or decrease) the expression of cancer marker genes. In some embodiments, candidate compounds are antisense agents (e.g., oligonucleotides) directed against cancer markers. See below for a discussion of antisense therapy. In other embodiments, candidate compounds are antibodies that specifically bind to a cancer marker of the present invention.

In one screening method, candidate compounds are evaluated for their ability to alter cancer marker expression by contacting a compound with a cell expressing a cancer marker and then assaying for the effect of the candidate compounds on expression. In some embodiments, the effect of candidate compounds on expression of a cancer marker gene is assayed for by detecting the level of cancer marker MRNA expressed by the cell. MRNA expression can be detected by any suitable method. In other embodiments, the effect of candidate compounds on expression of cancer marker genes is assayed by measuring the level of polypeptide encoded by the cancer markers. The level of polypeptide expressed can be measured using any suitable method, including but not limited to, those disclosed herein.

Specifically, the present invention provides screening methods for identifying modulators, i.e., candidate or test compounds or agents (e.g., proteins, peptides, peptidomimetics, peptoids, small molecules or other drugs) which bind to cancer markers of the present invention, have an inhibitory (or stimulatory) effect on, for example, cancer marker expression or cancer markers activity, or have a stimulatory or inhibitory effect on, for example, the expression or activity of a cancer marker substrate. Compounds thus identified can be used to modulate the activity of target gene products (e.g., cancer marker genes) either directly or indirectly in a therapeutic protocol, to elaborate the biological function of the target gene product, or to identify compounds that disrupt normal target gene interactions. Compounds which inhibit the activity or expression of cancer markers are useful in the treatment of proliferative disorders, e.g., cancer, particularly metastatic (e.g., androgen independent) prostate cancer.

In one embodiment, the invention provides assays for screening candidate or test compounds that are substrates of a cancer markers protein or polypeptide or a biologically active portion thereof. In another embodiment, the invention provides assays for screening candidate or test compounds that bind to or modulate the activity of a cancer marker protein or polypeptide or a biologically active portion thereof.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin Science 249:404-406 [1990]; Cwirla et al., Proc. NatI. Acad. Sci. 87:6378-6382 [1990]; Felici, J. Mol. Biol. 222:301 [1991]).

In one embodiment, an assay is a cell-based assay in which a cell that expresses a cancer marker protein or biologically active portion thereof is contacted with a test compound, and the ability of the test compound to the modulate cancer marker's activity is determined. Determining the ability of the test compound to modulate cancer marker activity can be accomplished by monitoring, for example, changes in enzymatic activity. The cell, for example, can be of mammalian origin.

The ability of the test compound to modulate cancer marker binding to a compound, e.g., a cancer marker substrate, can also be evaluated. This can be accomplished, for example, by coupling the compound, e.g., the substrate, with a radioisotope or enzymatic label such that binding of the compound, e.g., the substrate, to a cancer marker can be determined by detecting the labeled compound, e.g., substrate, in a complex.

Alternatively, the cancer marker is coupled with a radioisotope or enzymatic label to monitor the ability of a test compound to modulate cancer marker binding to a cancer markers substrate in a complex. For example, compounds (e.g., substrates) can be labeled with ¹²⁵I, ³⁵S ¹⁴C or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a cancer marker substrate) to interact with a cancer marker with or without the labeling of any of the interactants can be evaluated. For example, a microphysiometer can be used to detect the interaction of a compound with a cancer marker without the labeling of either the compound or the cancer marker (McConnell et al. Science 257:1906-1912 [1992]). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and cancer markers.

In yet another embodiment, a cell-free assay is provided in which a cancer marker protein or biologically active portion thereof is contacted with a test compound and the ability of the test compound to bind to the cancer marker protein or biologically active portion thereof is evaluated. Preferred biologically active portions of the cancer markers proteins to be used in assays of the present invention include fragments that participate in interactions with substrates or other proteins, e.g., fragments with high surface probability scores.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FRET) (see, for example, Lakowicz et al., U.S. Pat. No. 5,631,169; Stavrianopoulos et al., U.S. Pat. No. 4,968,103; each of which is herein incorporated by reference). A fluorophore label is selected such that a first donor molecule's emitted fluorescent energy will be absorbed by a fluorescent label on a second, ‘acceptor’ molecule, which in turn is able to fluoresce due to the absorbed energy.

Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in 1 5 the assay should be maximal. An FRET binding event can be conveniently measured through standard fluorometric detection means well known in the art (e.g., using a fluorimeter).

In another embodiment, determining the ability of the cancer marker protein to bind to a target molecule can be accomplished using real-time Biomolecular Interaction Analysis (BIA) (see, e.g., Sjolander and Urbaniczky, Anal. Chem. 63:2338-2345 [1991] and Szabo et al. Curr. Opin. Struct. Biol. 5:699-705 [1995]). “Surface plasmon resonance” or “BIA” detects biospecific interactions in real time, without labeling any of the interactants (e.g., BlAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize cancer markers, an anti-cancer marker antibody or its target molecule to facilitate separation of complexed from non-complexed forms of one or both of the proteins, as well as to accommodate automation of the assay. Binding of a test compound to a cancer marker protein, or interaction of a cancer marker protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase-cancer marker fusion proteins or glutathione-S-transferase/target fusion proteins can be adsorbed onto glutathione Sepharose beads (Sigma Chemical, St. Louis, Mo.) or glutathione-derivatized microtiter plates, which are then combined with the test compound or the test compound and either the non-adsorbed target protein or cancer marker protein, and the mixture incubated under conditions conducive for complex formation (e.g., at physiological conditions for salt and pH). Following incubation, the beads or microtiter plate wells are washed to remove any unbound components, the matrix immobilized in the case of beads, complex determined either directly or indirectly, for example, as described above.

Alternatively, the complexes can be dissociated from the matrix, and the level of cancer markers binding or activity determined using standard techniques. Other techniques for immobilizing either cancer markers protein or a target molecule on matrices include using conjugation of biotin and streptavidin. Biotinylated cancer marker protein or target molecules can be prepared from biotin-NHS (N-hydroxy-succinimide) using techniques known in the art (e.g., biotinylation kit, Pierce Chemicals, Rockford, EL), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-IgG antibody).

This assay is performed utilizing antibodies reactive with cancer marker protein or target molecules but which do not interfere with binding of the cancer markers protein to its target molecule. Such antibodies can be derivatized to the wells of the plate, and unbound target or cancer markers protein trapped in the wells by antibody conjugation. Methods for detecting such complexes, in addition to those described above for the GST-immobilized complexes, include immunodetection of complexes using antibodies reactive with the cancer marker protein or target molecule, as well as enzyme-linked assays which rely on detecting an enzymatic activity associated with the cancer marker protein or target molecule.

Alternatively, cell free assays can be conducted in a liquid phase. In such an assay, the reaction products are separated from unreacted components, by any of a number of standard techniques, including, but not limited to: differential centrifugation (see, for example, Rivas and Minton, Trends Biochem Sci 18:284-7 [1993]); chromatography (gel filtration chromatography, ion-exchange chromatography); electrophoresis (see, e.g., Ausubel et al., eds. Current Protocols in Molecular Biology 1999, J. Wiley: New York.); and immunoprecipitation (see, for example, Ausubel et al., eds. Current Protocols in Molecular Biology 1999, J. Wiley: New York). Such resins and chromatographic techniques are known to one skilled in the art (See e.g., Heegaard J. Mol. Recognit 11:141-8 [1998]; Hageand Tweed J. Chromatogr. Biomed. Sci. Appl 699:499-525 [1997]). Further, fluorescence energy transfer may also be conveniently utilized, as described herein, to detect binding without further purification of the complex from solution.

The assay can include contacting the cancer markers protein or biologically active portion thereof with a known compound that binds the cancer marker to form an assay mixture, contacting the assay mixture with a test compound, and determining the ability of the test compound to interact with a cancer marker protein, wherein determining the ability of the test compound to interact with a cancer marker protein includes determining the ability of the test compound to preferentially bind to cancer markers or biologically active portion thereof, or to modulate the activity of a target molecule, as compared to the known compound.

To the extent that cancer markers can, in vivo, interact with one or more cellular or extracellular macromolecules, such as proteins, inhibitors of such an interaction are useful. A homogeneous assay can be used can be used to identify inhibitors.

For example, a preformed complex of the target gene product and the interactive cellular or extracellular binding partner product is prepared such that either the target gene products or their binding partners are labeled, but the signal generated by the label is quenched due to complex formation (see, e.g., U.S. Pat. No. 4,109,496, herein incorporated by reference, that utilizes this approach for immunoassays). The addition of a test substance that competes with and displaces one of the species from the preformed complex will result in the generation of a signal above background. In this way, test substances that disrupt target gene product-binding partner interaction can be identified. Alternatively, cancer markers protein can be used as a “bait protein” in a two-hybrid assay or three-hybrid assay (see, e.g., U.S. Pat. No. 5,283,317; Zervos et al., Cell 72:223-232 [1993]; Madura et al., J. Biol. Chem. 268.12046-12054 [1993]; Bartel et al., Biotechniques 14:920-924 [1993]; Iwabuchi et al., Oncogene 8:1693-1696 [1993]; and Brent WO94/10300; each of which is herein incorporated by reference), to identify other proteins, that bind to or interact with cancer markers (“cancer marker-binding proteins” or “cancer marker-bp”) and are involved in cancer marker activity. Such cancer marker-bps can be activators or inhibitors of signals by the cancer marker proteins or targets as, for example, downstream elements of a cancer markers-mediated signaling pathway.

Modulators of cancer markers expression can also be identified. For example, a cell or cell free mixture is contacted with a candidate compound and the expression of cancer marker mRNA or protein evaluated relative to the level of expression of cancer marker mRNA or protein in the absence of the candidate compound. When expression of cancer marker mRNA or protein is greater in the presence of the candidate compound than in its absence, the candidate compound is identified as a stimulator of cancer marker mRNA or protein expression. Alternatively, when expression of cancer marker mRNA or protein is less (i.e., statistically significantly less) in the presence of the candidate compound than in its absence, the candidate compound is identified as an inhibitor of cancer marker mRNA or protein expression. The level of cancer markers mRNA or protein expression can be determined by methods described herein for detecting cancer markers mRNA or protein.

A modulating agent can be identified using a cell-based or a cell free assay, and the ability of the agent to modulate the activity of a cancer markers protein can be confirmed in vivo, e.g., in an animal such as an animal model for a disease (e.g., an animal with prostate cancer or metastatic prostate cancer; or an animal harboring a xenograft of a prostate cancer from an animal (e.g., human) or cells from a cancer resulting from metastasis of a prostate cancer (e.g., to a lymph node, bone, or liver), or cells from a prostate cancer cell line.

This invention further pertains to novel agents identified by the above-described screening assays (See e.g., below description of cancer therapies). Accordingly, it is within the scope of this invention to further use an agent identified as described herein (e.g., a cancer marker modulating agent, an antisense cancer marker nucleic acid molecule, a siRNA molecule, a cancer marker specific antibody, or a cancer marker-binding partner) in an appropriate animal model (such as those described herein) to determine the efficacy, toxicity, side effects, or mechanism of action, of treatment with such an agent. Furthermore, novel agents identified by the above-described screening assays can be, e.g., used for treatments as described herein.

IV. Transgenic Animals Expressing Cancer Marker Genes

The present invention contemplates the generation of transgenic animals comprising an exogenous cancer marker gene of the present invention or mutants and variants thereof (e.g., truncations or single nucleotide polymorphisms). In preferred embodiments, the transgenic animal displays an altered phenotype (e.g., increased or decreased presence of markers) as compared to wild-type animals. Methods for analyzing the presence or absence of such phenotypes include but are not limited to, those disclosed herein. In some preferred embodiments, the transgenic animals further display an increased or decreased growth of tumors or evidence of cancer.

The transgenic animals of the present invention find use in drug (e.g., cancer therapy) screens. In some embodiments, test compounds (e.g., a drug that is suspected of being useful to treat cancer) and control compounds (e.g., a placebo) are administered to the transgenic animals and the control animals and the effects evaluated.

The transgenic animals can be generated via a variety of methods. In some embodiments, embryonal cells at various developmental stages are used to introduce transgenes for the production of transgenic animals. Different methods are used depending on the stage of development of the embryonal cell. The zygote is the best target for micro-injection. In the mouse, the male pronucleus reaches the size of approximately 20 micrometers in diameter that allows reproducible injection of 1-2 picoliters (pl) of DNA solution. The use of zygotes as a target for gene transfer has a major advantage in that in most cases the injected DNA will be incorporated into the host genome before the first cleavage (Brinster et al., Proc. Natl. Acad. Sci. USA 82:4438-4442 [1985]). As a consequence, all cells of the transgenic non-human animal will carry the incorporated transgene. This will in general also be reflected in the efficient transmission of the transgene to offspring of the founder since 50% of the germ cells will harbor the transgene. U.S. Pat. No. 4,873,191 describes a method for the micro-injection of zygotes; the disclosure of this patent is incorporated herein in its entirety.

In other embodiments, retroviral infection is used to introduce transgenes into a non-human animal. In some embodiments, the retroviral vector is utilized to transfect oocytes by injecting the retroviral vector into the perivitelline space of the oocyte (U.S. Pat. No. 6,080,912, incorporated herein by reference). In other embodiments, the developing non-human embryo can be cultured in vitro to the blastocyst stage. During this time, the blastomeres can be targets for retroviral infection (Janenich, Proc. Natl. Acad. Sci. USA 73:1260 [1976]). Efficient infection of the blastomeres is obtained by enzymatic treatment to remove the zona pellucida (Hogan et al., in Manipulating the Mouse Embryo, Cold Spring Harbor Laboratory Press, Cold Spring Harbor, N.Y. [1986]). The viral vector system used to introduce the transgene is typically a replication-defective retrovirus carrying the transgene (Jahner et al., Proc. Natl. Acad Sci. USA 82:6927 [1985]). Transfection is easily and efficiently obtained by culturing the blastomeres on a monolayer of virus-producing cells (Stewart, et al., EMBO J., 6:383 [1987]). Alternatively, infection can be performed at a later stage. Virus or virus-producing cells can be injected into the blastocoele (Jahner et al., Nature 298:623 [1982]). Most of the founders will be mosaic for the transgene since incorporation occurs only in a subset of cells that form the transgenic animal. Further, the founder may contain various retroviral insertions of the transgene at different positions in the genome that generally will segregate in the offspring. In addition, it is also possible to introduce transgenes into the germline, albeit with low efficiency, by intrauterine retroviral infection of the midgestation embryo (Jahner et al., supra [1982]). Additional means of using retroviruses or retroviral vectors to create transgenic animals known to the art involve the micro-injection of retroviral particles or mitomycin C-treated cells producing retrovirus into the perivitelline space of fertilized eggs or early embryos (PCT International Application WO 90/08832 [1990], and Haskell and Bowen, Mol. Reprod. Dev., 40:386 [1995]).

In other embodiments, the transgene is introduced into embryonic stem cells and the transfected stem cells are utilized to form an embryo. ES cells are obtained by culturing pre-implantation embryos in vitro under appropriate conditions (Evans et al., Nature 292:154 [1981]; Bradley et al., Nature 309:255 [1984]; Gossler et al., Proc. Acad. Sci. USA 83:9065 [1986]; and Robertson et al., Nature 322:445 [1986]). Transgenes can be efficiently introduced into the ES cells by DNA transfection by a variety of methods known to the art including calcium phosphate co-precipitation, protoplast or spheroplast fusion, lipofection and DEAE-dextran-mediated transfection. Transgenes may also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal (for review, See, Jaenisch, Science 240:1468 [1988]). Prior to the introduction of transfected ES cells into the blastocoel, the transfected ES cells may be subjected to various selection protocols to enrich for ES cells which have integrated the transgene assuming that the transgene provides a means for such selection. Alternatively, the polymerase chain reaction may be used to screen for ES cells that have integrated the transgene. This technique obviates the need for growth of the transfected ES cells under appropriate selective conditions prior to transfer into the blastocoel.

In still other embodiments, homologous recombination is utilized to knock-out gene function or create deletion mutants (e.g., truncation mutants). Methods for homologous recombination are described in U.S. Pat. No. 5,614,396, incorporated herein by reference.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); l or L (liters); ml (milliliters); μl (microliters); cm (centimeters); mm (millimeters); μm (micrometers); nm (nanometers); and ° C. (degrees Centigrade).

### Example 1

### A. Experimental Procedures

High-throughput Immunoblot Analysis

Tissues utilized were from the radical prostatectomy series at the University of Michigan and from the Rapid Autopsy Program, which are both part of University of Michigan Prostate Cancer Specialized Program of Research Excellence (S.P.O.R.E.) Tissue Core. Institutional Review Board approval was obtained to procure and analyze the tissues used in this study. To develop the tissue extract pools the following frozen tissue blocks were identified: 5 each of benign prostate tissues, clinically localized prostate cancer (3 were Gleason pattern 3+3, and 1 each of Gleason 3+4 and 4+3), and hormone-refractory metastatic tissues (liver, lymph node, lung, dura and soft tissue metastasis) (Shah et al., 2004, Cancer Res 64, 9209-9216). Based on examination of the frozen sections of each tissue block, specimens were grossly dissected maintaining at least 90% of the tissue of interest. Total proteins were extracted from each tissue by homogenizing samples in boiling lysis buffer (contains 10 mM Tris-HCI pH 7.5 containing 1% SDS and 100 μmolar sodium orthovanadate). The protein concentrations were determined by using Biorad DC (Detergent Compatible) protein assay kit (Biorad, Hercules, Calif.). Extracts from each of the 5 specimens were combined equally to establish a pool. One hundred micrograms of protein from each tissue extract pool was boiled in sample buffer and subjected to 4-15% preparative SDS-PAGE and transferred to PVDF (Amersham Biosciences Corp, Piscataway, N.J.). The membranes were incubated for 1 hour in blocking buffer (Tris-buffered saline with 0.1% Tween [TBS-T] and 5% nonfat dry milk).

Fifty-two antibodies and 4 control antibodies could be assessed in each Miniblotter system (Immunetics, Cambridge, Mass.). Antibodies (n=524) at various dilutions (60 μL total volume in TBS-T and 5% milk were loaded in the miniblotter system and incubated with the membranes for 2 hours. After washing three times with TBS-T buffer, the membranes were incubated with horseradish peroxidase-linked secondary IgG antibody (mouse, rabbit or goat depending on the primary antibody used) (Amersham Biosciences Corp, Piscataway, N.J.) at 1:5000 for 2 hour at room temperature. The signals were visualized with the ECL detection system (Amersham Pharmacia biotech, Piscataway, N.J.) and autoradiography.

To supplement the number of proteins analyzed, the same extracts were analyzed using two commercial service providers, BD and Kinexus. Power blot high-throughput immunoblots were carried out by BD biosciences (San Diego, Calif.) (Malakhov et al., 2003, J Biol Chem 278, 16608-16613). Briefly, samples were separated on a 4-15% gradient SDS-polyacrylamide gel and transferred to Immobilon-P membrane (Millipore, Bedford, Mass.). After transfer, the membrane is dried and re-wet in methanol. The membrane is then incubated for one hour with blocking buffer (LI-COR, Lincoln, Nebr. U.S.A.) and is clamped with a western blotting manifold that provides 40 channels across the membrane. In each channel, a complex antibody cocktail is added and allowed to hybridize for one hour at 37° C. The blot is removed from the manifold, washed and hybridized for 30 minutes at 37° C. with secondary goat anti-mouse conjugated to Alexa680 fluorescent dye (Molecular Probes, Eugene, Oreg.). The membrane was washed, dried and scanned using the Odyssey Infrared Imaging System (LI-COR, Lincoln, Nebr. U.S.A.). For phosphoptotein analyses samples were prepared according to the instructions provided by Kinexus, Inc. Signals from antibodies generating an immunoreactive band at the expected molecular weight were evaluated visually and quantitated by densitometry or scanned using the Odyssey Infrared Imaging System (LI-COR). From the immunoreactive bands assessed, visually qualified signals were selected for further validation. Visually qualified proteins that were over-expressed were coded red and given a value of 1, under-expressed proteins were coded blue and set at a value of −1, and white was used for unchanged proteins.

Conventional Immunoblot Validation

Validation immunoblots for selected proteins in different functional classes were carried out using 4-15% linear gradient SDS-PAGE gels. Tissue lysates from 3 to 4 benign, 5 clinically localized and 5 metastatic prostate cancers were separated on a SDSPAGE and transferred to PVDF membrane. The immunoblot was carried out using different antibodies and at specific dilutions.

Tissue Microarray Analysis (TMA)

A prostate cancer progression TMA composed of benign prostate tissue, clinically localized prostate cancer, and hormone refractory metastatic prostate cancer was developed. These cases came from well fixed radical prostatectomy specimens as described previously (Rubin et al., 2002, Jama 287, 1662-1670). Replicate tissue samples were placed in geographically distinct areas of the TMA in order to evaluate reproducibility within the same TMA based on location. Total 216 tissue samples were collected from 51 patients.

Pre-treatment conditions and incubation times were worked up for each antibody optimizing signal to noise ratio. The TMA was soaked in xylene overnight to remove adhesive tape used for its construction. Pre-treatments varied depending on the optimal conditions. Primary antibodies were incubated before washing. Secondary antimouse or anti-rabbit antibodies avidin-conjugated were applied before washing. Enyzmatic reaction was completed using a strepavidin biotin detection kit (DakoCytomation, Carpinteria, Calif.).

Protein expression was determined using a validated scoring method (Dhanasekaran et al., 2001, Nature 412, 822-826; Rubin et al., 2002, supra; Varambally et al., 2002, Nature 419, 624-629) where staining was evaluated for intensity and the percentage of cells staining positive. Benign epithelial glands and prostate cancer cells were scored for staining intensity on a 4 tiered system ranging from negative to strong expression. An estimate of the number of cells staining positive over background was evaluated for each 0.6 mm core. In cases where benign tissue and cancer were present, only one or the other tissue type was evaluated for purposes of analysis.

Hierarchical clustering on samples and proteins was carried out after data normalization. Measurements were averaged for duplicated samples in the same patient, base 2 log-transformed, and each protein was normalized so that its mean across all of samples equaled zero and the variance was 1.

Integrative Molecular Analysis

To map the antibodies and their respective protein targets, the official gene names were obtained from the NCBI Locuslink for antibody/protein lists. To complement protein levels, transcriptome data was assembled from 8 publicly available prostate cancer gene expression datasets (Dhanasekaran et al., 2001, supra; Lapointe et al., 2004, Proc Natl Acad Sci USA 101, 811-816; LaTulippe et al., 2002, Cancer Res 62, 4499-4506; Luo et al., 2001, Cancer Res 61, 4683-4688; Luo et al., 2002b, Mol Carcinog 33, 25-35; Singh et al., 2002, Cancer Cell 1, 203-209; Welsh et al., 2001, Cancer Res 61, 5974-5978; Yu et al., 2004, J Clin Oncol 22, 2790-2799) and each probe was mapped to Unigene Build #173 (Table S3). Expression values from multiple clones or probe sets mapping to the same Unigene Cluster ID were averaged. Each gene in each study was normalized across samples so that the mean equaled zero and the standard deviation equaled to 1. Missing data was imputed by the k-nearest neighbors (k=5) imputation approach (Troyanskaya et al., 2001, Bioinformatics 17, 520-525).

Eight prostate cancer profiling studies were included in the analysis of clinically localized prostate cancer relative to benign prostate tissue, while only 4 studies were included in the analysis of metastatic prostate cancer vs. localized prostate cancer due to the availability of metastatic samples in those studies. Genes that were only found in one-fourth of studies or less were excluded, leading to 483 genes involved in the former analysis and 494 involved in the latter analysis. A one-sided permutation t-test was conducted per gene per study using the multtest package in R 2.0. A gene was considered differentially expressed if its p-value was less than 0.05 without adjustment for multiple testing. An mRNA transcript alteration was considered “concordant” with a proteomic alteration if a majority of the microarray profiling studies (at least 50%) showed the same qualitative differential (increased, decreased, or unchanged) as the highthroughput immunoblot approach. The gene/proteins were then assigned to concordant and discordant groups based on this criterion.

Clinical Outcomes Analysis

Six different cancer profiling studies (Bhattacharjee et al., 2001, Proc Natl Acad Sci USA 98, 13790-13795; Freije et al., 2004, Cancer Res 64, 6503-6510; Glinsky et al., 2004, J Clin Invest 113, 913-923; Huang et al., 2003, Lancet 361, 1590-1596; van 't Veer et al., 2002, Nature 415, 530-536; Yu et al., 2004, supra) were used for evaluation of prognostic value of these concordant genes. Detailed study information is shown in Table 3. Average linkage hierarchical clustering using an uncentered correlation similarity metric was used to identify two main clusters of clinically localized prostate cancer samples based on the 44 concordant MRNA transcripts that were qualitatively concordant with protein expression in the Yu et al. (Yu et al., 2004, supra) study (only 44 out of 50 of the concordant signature were assessed on these arrays). Kaplan-Meier survival analysis of cluster-defined subgroups was then conducted and the log-rank test was used to calculate the statistical significance of difference between the two subgroups (SPSS 11.5). High-/low-risk labels were then assigned to each group. A permutation test was performed to evaluate the significance of this “lethal” concordant signature. 1000 random sets of 44 genes from the Yu et al. data set were selected and used to carry out 1000 independent clusterings of the primary prostate cancer samples. Each grouping was subjected each grouping to Kaplan-Meier survival analysis.

To validate the prognostic association of the 44-gene concordant signature, an independent (clinically localized) prostate cancer gene expression dataset from Glinsky et al. (Glinsky et al., 2004, supra) was used. The Yu et al. clustering functioned as the “training set” to define high-/low-risk groups. Each patient of the Glinsky et al. study was classified into one of the two groups based on k-nearest neighbor classification (k=3) using as the similarity metric the Pearson correlation coefficient in the space of the significant genes from the Yu et al. dataset. Each “test” sample was then classified into high-/low-risk group based on which cluster the majority of the test patient's nearest neighbors belonged. Kaplan-Meier survival curves were plotted for the two groupings. This “lethal” signature was then refined by reducing the number of genes involved. By using Yu et al. study as a training set, the concordant genes were ranked by univariate cox model. Again, the clustering procedure was used to identify two clusters based on the top number of genes (ranging from 5 to 44). The Glinsky et al. study was then used as a validation set to verify performance of the refined signature by k-nearest neighbors (k=3) prediction analysis.

The generality of this “lethal” signature was evaluated by using other solid tumor datasets. The signature was applied to two breast cancer (Huang et al., 2003, supra)-(van 't Veer et al., 2002, supra), one lung cancer (Bhattacharjee et al., 2001, supra) and one glioma (Freije et al., 2004, supra) gene expression study. Clustering was used to identify two main clusters for patients in each study and Kaplan-Meier survival analysis was conducted to evaluate the statistical significance of differences between survival curves.

Multivariable Analysis

A Cox proportional-hazards regression model was used to carry out the multivariate analysis. The dichotomized values of the 44-gene lethal signature, preoperative PSA, Gleason sum score from prostatectomy specimens, preoperative clinical stage, age, and status of surgical margins were included as covariates. The calculation was performed with the R 2.0 statistical package.

Pathway Analysis

To better understand the biological pathways at work in the concordant and discordant signature, the association of these genes with gene sets defined by Gene Ontology and Transfac analysis (Rhodes et al., 2005, Nat Genet 37, 579-583) was investigated. The overlap of the signature with each gene set was counted and the significance of the overlap was evaluated with Fisher's exact test.

### B. Results and Discussion

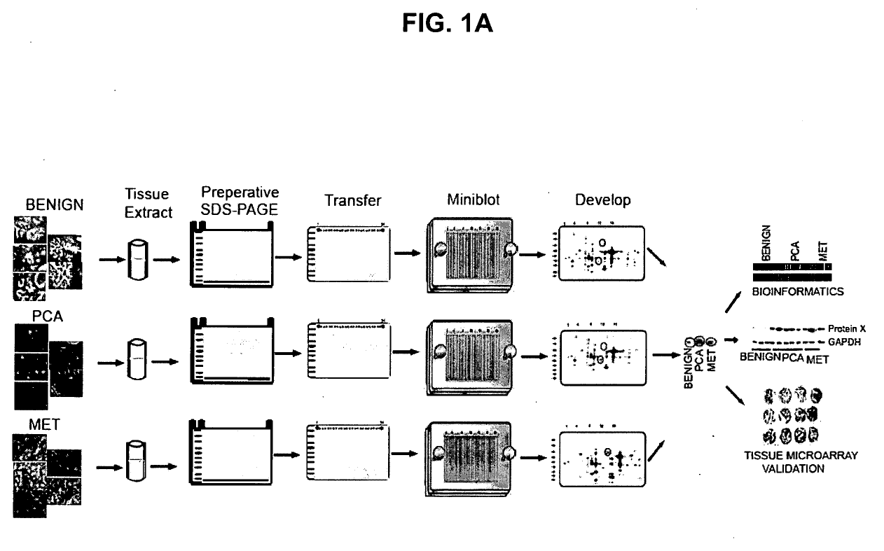

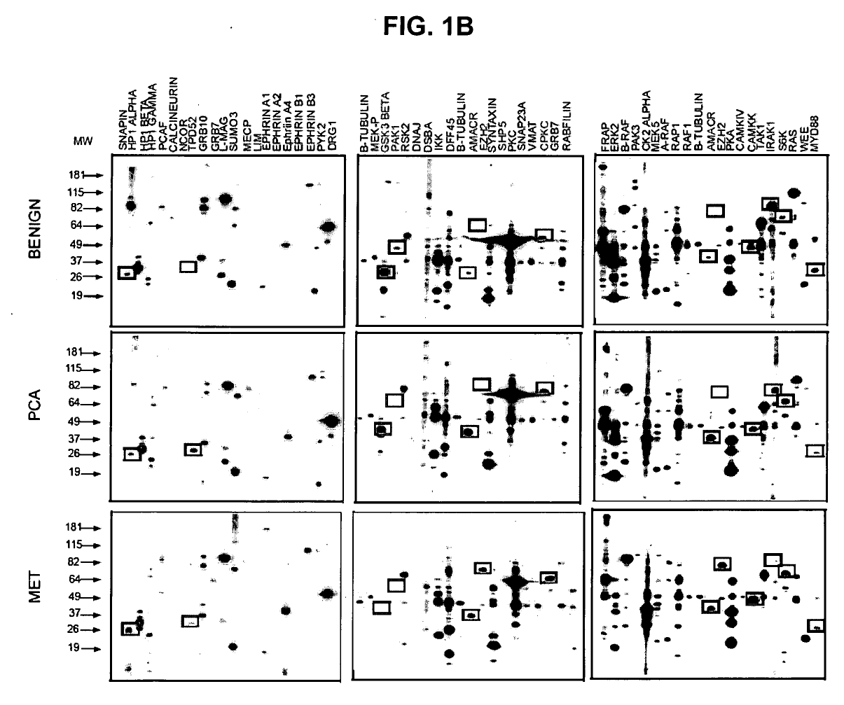

In order to derive a first approximation of the prostate cancer proteome, high-throughput immunoblot analysis was utilized. This method allowed for the screening of pooled tissue extracts for qualitative levels of hundreds of proteins (and post-translational modifications) using commercially available antibody reagents. The basic approach is illustrated in FIG. 1A. Extracts from five tissue specimens of benign prostate, clinically localized prostate cancer and metastatic prostate cancer from distinct patients were pooled. Each of the 3 pools of tissue extracts were run on preparative SDS-PAGE gels, transferred to PVDF, and incubated with 1484 antibodies using a miniblot apparatus. FIG. 1B displays representative data using the high-throughput immunoblot approach. Known proteomics alterations in prostate cancer progression such as EZH2 (Varambally et al., 2002, Nature 419, 624-629) and AMACR (Jiang et al., 2001, Am J Surg Pathol 25, 1397-1404; Luo et al., 2002a, Cancer Res 62, 2220-2226; Rubin et al., 2002, supra) are highlighted in red while novel associations such as GSK-3beta and IRAK1 are highlighted in green. To further increase the number of proteins analyzed, an analogous high-throughput immunoblot methodology provided by commercial services was utilized (See Methods). Thus, in total 1484 antibodies against 1354 distinct proteins or post-translational modifications were assessed. Of these antibodies, 521 detected a band of the expected molecular weight in at least one of the pooled extracts. Antibodies that did not detect the correct molecular weight protein product may represent lack of antibody sensitivity (or poor quality antibody) or absence of protein expression in prostate tissues.

To validate that the proteomic alterations identified by this screen occur in individual tissue extracts (as opposed to pooled extracts), 86 proteins were analyzed by conventional immunoblot analysis using 4-5 tissue extracts per class. In order to evaluate the proteomics alterations in situ, high-density tissue microarrays were utilzed.

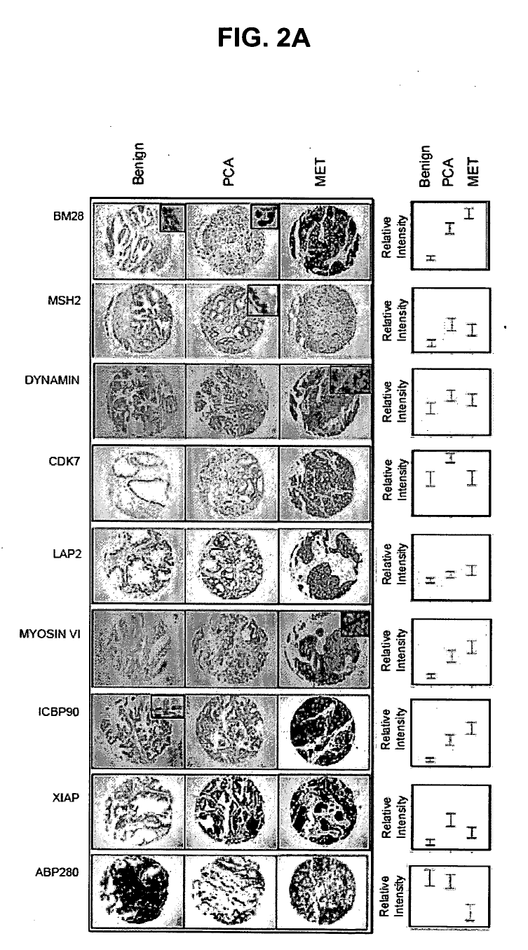

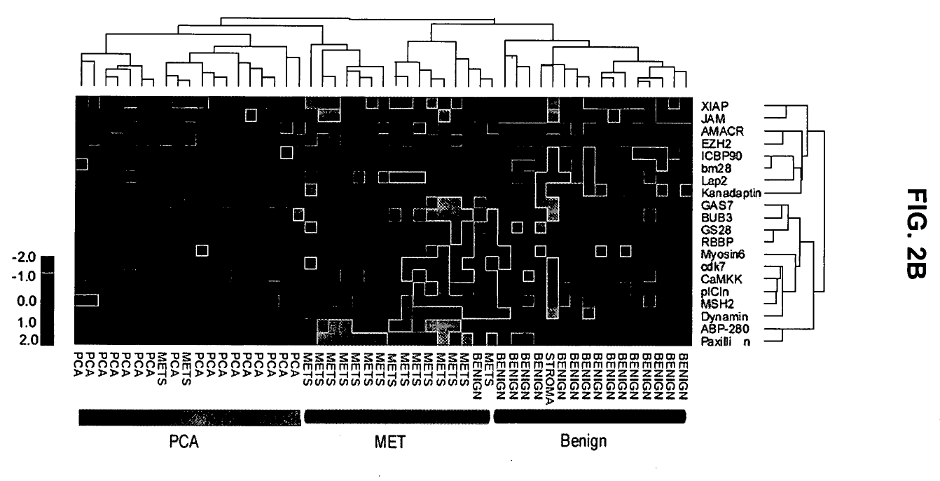

As only a subset of the identified proteins have antibodies that are compatible with immunohistochemical analysis, a single tissue microarray containing 216 specimens from 51 cases was stained using twenty of these IHC-compatible antibodies. Representative tissue microarray elements are shown in FIG. 2A. Each tissue microarray element was evaluated by a pathologist and scored for staining (scale of 1-4) as per cell type considered (e.g., epithelial, stromal etc. . . . ). Using an in situ technique such as evaluation by immunohistochemistry allowed us to distinguish stromal versus epithelial expressed proteins. In general, proteins that demonstrated a decrease in expression in the metastatic tumors most often were stromally expressed proteins. As the amount of stroma per unit area decreases with tumor progression, metastatic samples demonstrated a parallel decrease in protein expression of paxillin and ABP-280, among others. In order to visualize and cluster the tissue microarray data (Nielsen et al., 2003, Am J Pathol 163, 1449-1456), the qualitative evaluations were log transformed and normalized.

Similar to gene expression analyses (Eisen et al., 1998, Proc Natl Acad Sci USA 95, 14863-14868; Perou et al., 2000, Nature 406, 747-752), unsupervised hierarchical clustering of the data revealed that the in situ protein levels could be used to accurately classify prostate samples as benign, clinically localized prostate cancer, or metastatic disease (FIG. 2B).

This high-throughput immunoblotting of prostate extracts led to the identification of a several known and previously unknown proteomic alterations in prostate cancer. The proteomic alterations identified fall into a range of functional taxonomy including kinases and phosphatases, cell growth and apoptosis proteins, chromatin regulators, proteases, and proteins involved in cell structure and motility. For example, previous studies have shown that the anti-apoptosis protein, XIAP (Krajewska et al., 2003, Clin Cancer Res 9, 4914-4925), the racemase AMACR (Jiang et al., 2001, supra; Luo et al., 2002a, supra; Rubin et al., 2002, supra) and the Polycomb Group protein EZH2 (Varambally et al., 2002, supra) are dysregulated in prostate cancer progression. Novel associations (increases or decreases in protein expression) with prostate cancer progression identified by this screen include the E2 ubiquitin ligase UBc9, the cytosolic phosphoprotein stathmin, the death receptor DR3, and the Aurora A kinase (STK15), among others.

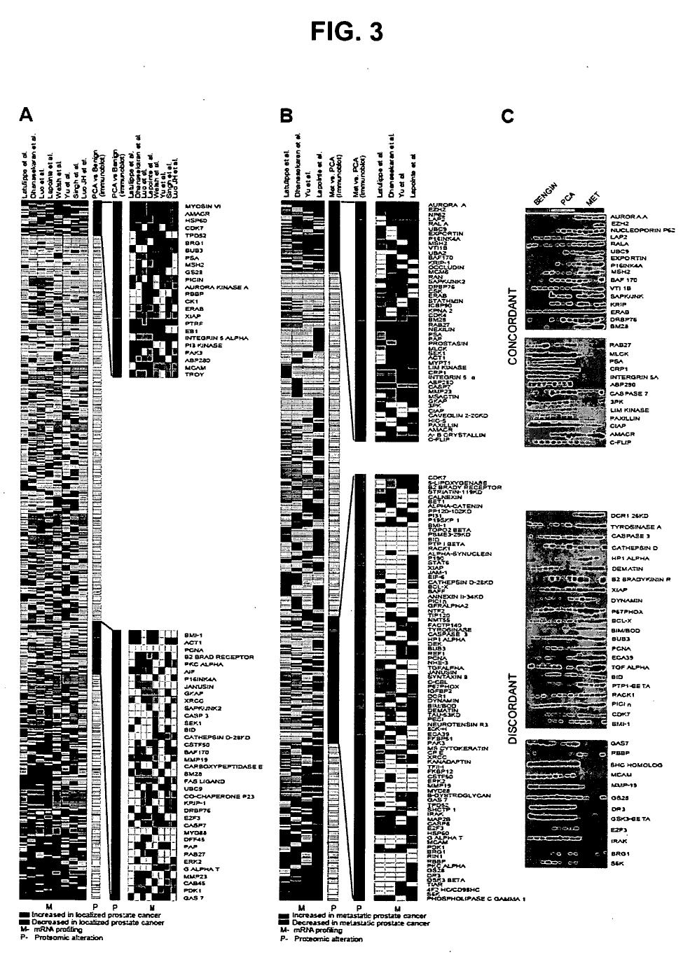

Having amassed this compendium of proteomic alterations in prostate cancer progression, the general concordance with the prostate cancer transcriptome was examined. An integrative model to incorporate qualitative proteomic alterations as assessed by high-throughput immunoblotting (but applicable to other proteomic technologies), with transcriptomic data derived from 8 prostate cancer gene expression studies was developed (FIG. 3). As both the genomic and proteomic approach involve analysis of grossly dissected tissues, this facilitates molecular comparisons to be made. The high-throughput immunoblot analysis of benign prostate, clinically localized prostate cancer and metastatic disease yielded 521 proteins of the expected molecular weight.

Immunoreactive bands in each of the three tissue extracts were assessed and comparisons were made between benign tissue and clinically localized prostate cancer (FIG. 3A) and between clinically localized prostate cancer and metastatic disease (FIG. 3B). Visually qualified proteins that were over-expressed were coded red, under-expressed proteins were coded blue, and unchanged proteins were coded white. Based on this analysis, 64 proteins were dysregulated in clinically localized prostate cancer relative to benign prostate tissue, while 156 proteins were dysregulated between metastatic disease relative to clinically localized prostate cancer.

The set of quantifiable proteins (n=521) was then mapped to the NCBI Locus link database to identify each corresponding gene. Data for mRNA was extracted for these genes using 8 publicly available prostate cancer gene expression data sets (Dhanasekaran et al., 2001, supra; Lapointe et al., 2004, supra; LaTulippe et al., 2002, supra; Luo et al., 2001, Cancer Res 61, 4683-4688; Luo et al., 2002b, supra; Singh et al., 2002, supra; Welsh et al., 2001, supra; Yu et al., 2004, supra). Over 90% of the genes were represented in at least one microarray study allowing for integrative analysis to be performed. Eight of the prostate profiling studies made a comparison between clinically localized prostate cancer and benign tissue, while only four of these made a comparison between clinically localized disease and metastatic disease. Genes that can only be found in one-fourth of studies or less were excluded, leading to 483 genes involved in the former comparison and 494 involved in the latter comparison. Since over and under-expressed genes were assessed separately, a one-sided t test was conducted per each gene per each profiling study (See Methods). As with the proteomic approach, comparisons between benign and clinically localized prostate cancer (FIG. 3A) and localized disease and metastatic disease (FIG. 3B) were made. If an mRNA transcript was significantly over-expressed in a particular study it was coded red, under-expressed transcripts were coded blue, and white was used for unchanged transcripts.

FIG. 3 presents the integrative proteomic and genomic analysis of prostate cancer progression. An mRNA transcript alteration was considered “concordant” with a proteomic alteration if a majority of the microarray profiling studies (at least 50%) showed the same qualitative differential (increased, decreased, or unchanged) as the highthroughput immunoblot approach. According to these criteria, 290 (60.0%) out of 483 mRNA transcripts were concordant with protein levels in clinically localized prostate cancer relative to benign prostate tissue. Similarly, 293 (59.3%) out of 494 mRNA transcripts were concordant with protein levels in metastatic prostate cancer relative to clinically localized disease. Thus, similar to studies done in yeast (Griffin et al., 2002, Mol Cell Proteomics 1, 323-333; Washburn et al., 2003, Proc Natl Acad Sci USA 100, 3107-3112), bacteria (Baliga et al., 2002, Proc Natl Acad Sci USA 99, 14913-14918), and cell lines (Tian et al., 2004, Mol Cell Proteomics 3, 960-969), there was only weak concordance between protein and mRNA levels in prostate cancer progression.

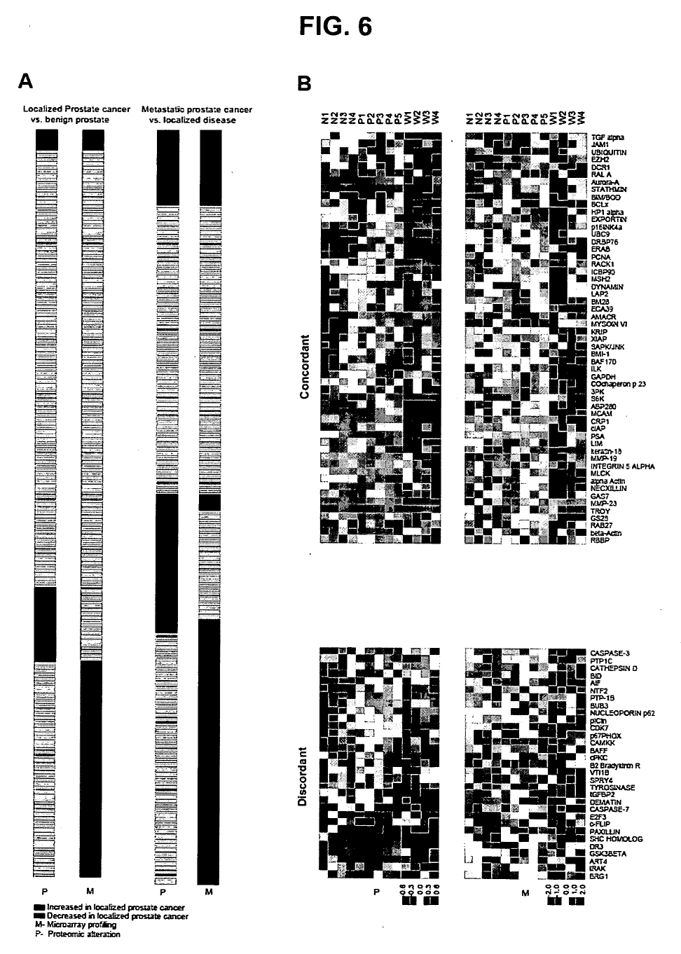

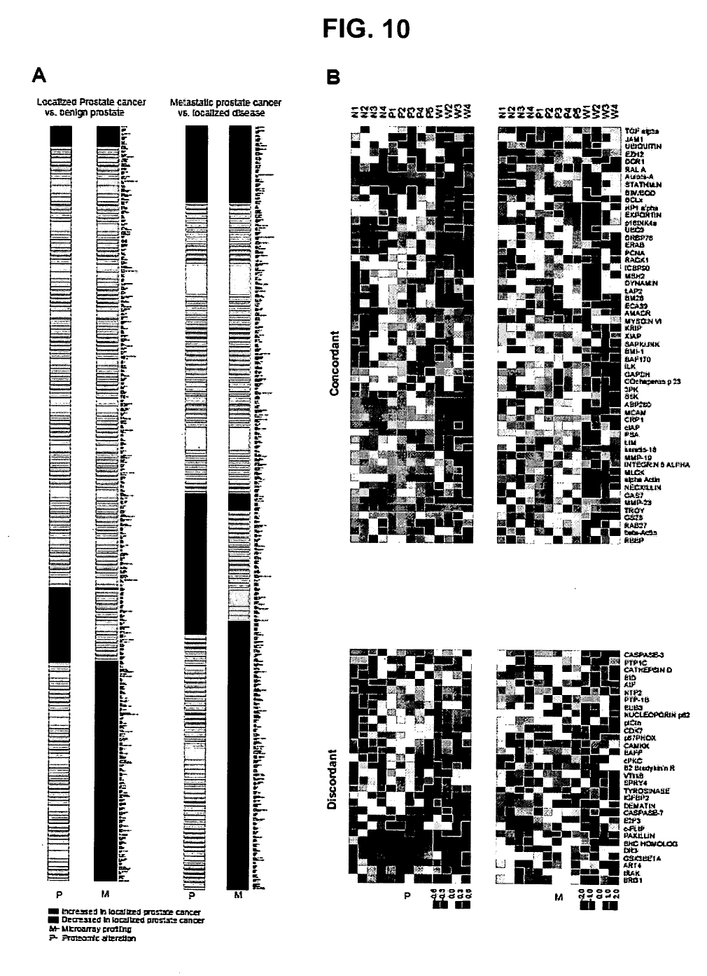

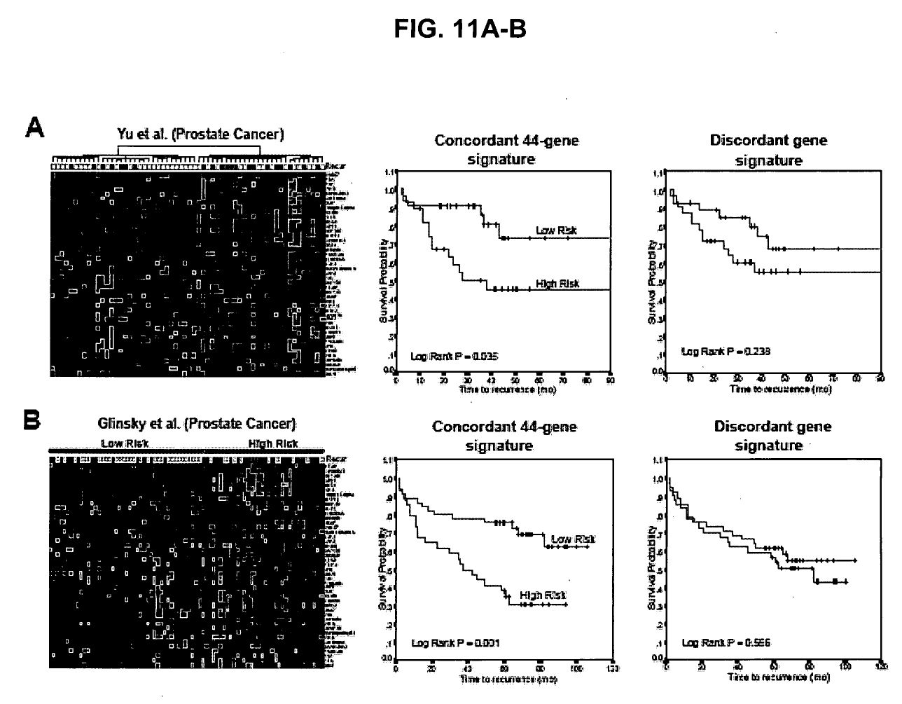

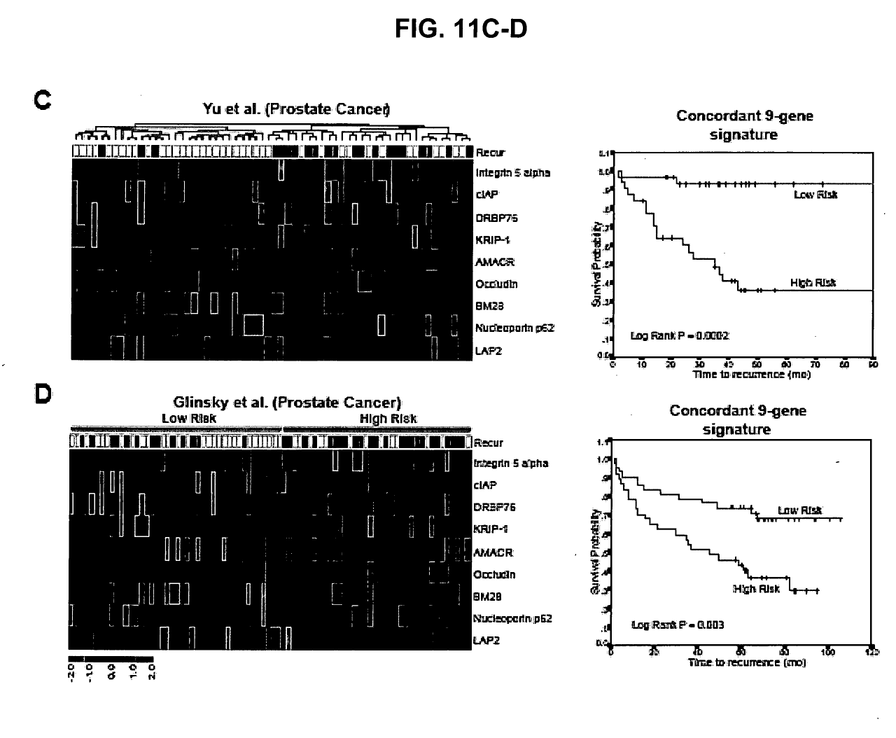

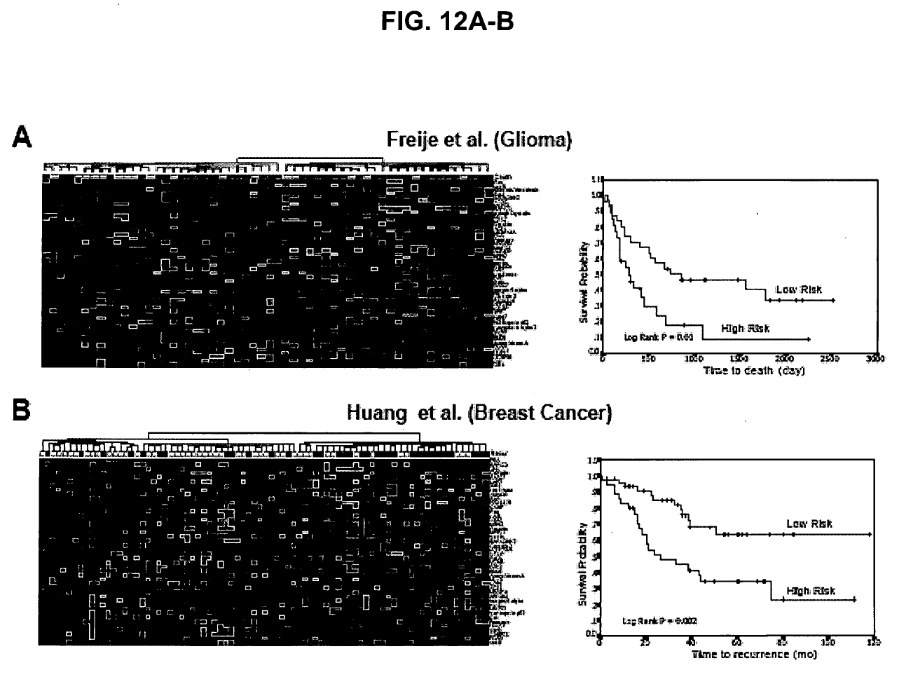

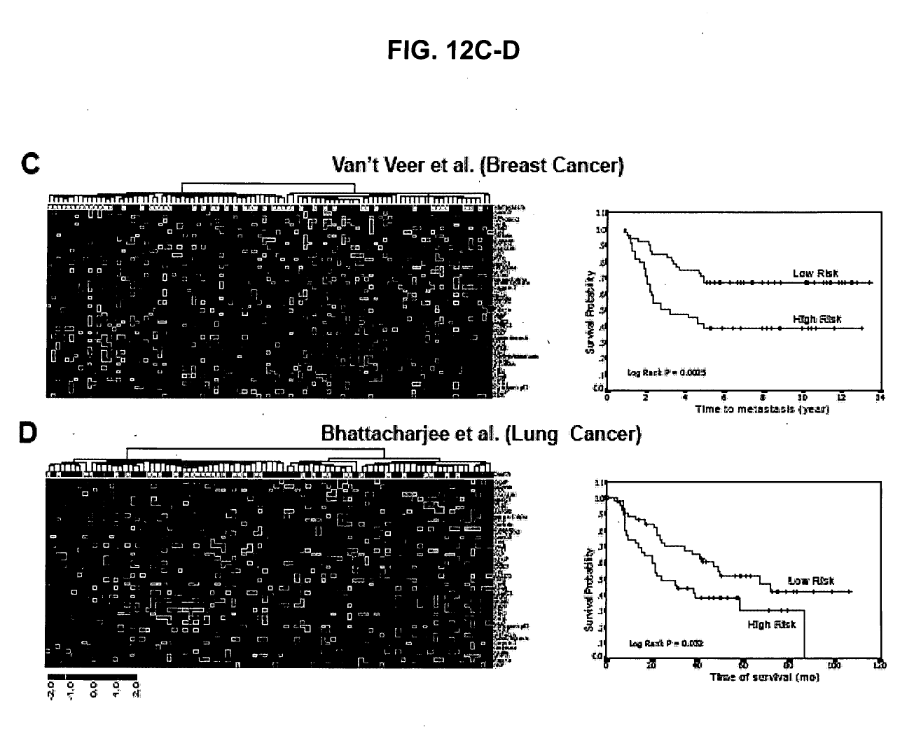

To further explore the poor concordance observed between protein and metadata from transcriptomic analyses, the pooled samples were profiled as well as the individual samples that comprised the pools on Affymetrix HG-U133 plus 2 microarrays. The same integrative analysis was carried out to examine the concordant relationship between the protein alterations observed in the pooled tissues by immunoblotting and transcript alterations observed in the corresponding pooled and individual tissues. The individual samples were included in order to calculate statistical significance for transcript alterations. Similar or even lower concordance was observed between protein and transcript (61.91% concordance in clinically localized prostate cancer relative to benign prostate tissue, and 47.96 % for metastatic prostate cancer relative to clinically localized disease, FIG. 6A, FIG. 10A).

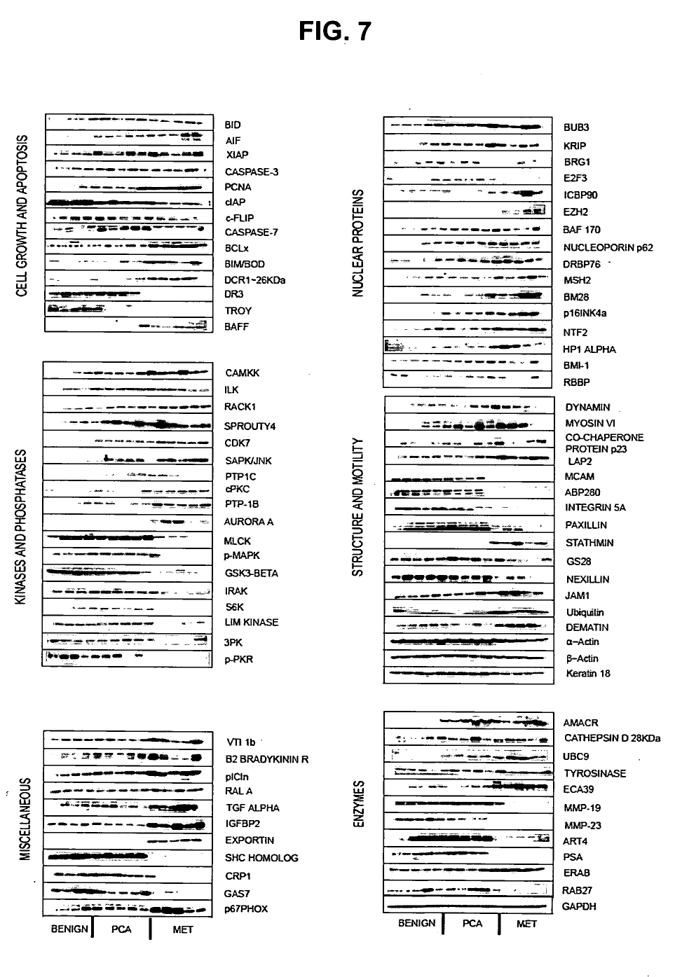

The protein and mRNA concordance in individual samples was also investigated. The 86 proteins identified as outliers in the larger high-throughput screen (see FIG. 7) were utilized. The immunoblot intensities were semi-quantitated and correlation coefficients were calculated for each protein (see Experimental Procedures). A total 55 out of 86 proteins were observed to a have a positive correlation with mRNA, which led to 64.0% concordance between proteins and transcripts (FIG. 6B). On sub classification, a concordance of 54.7% and 66.3% in case of localized prostate cancer relative to benign prostate tissues and the metastatic disease relative to localized prostate cancer respectively was observed.

This proteomic screen identified proteins that are altered from benign prostate to clinically localized prostate cancer and a distinct set of alterations between clinically localized disease to metastatic disease. The transition from clinically localized to metastatic disease was next investigated. As the metastatic tissues analyzed in this study are androgen-independent (Shah et al., 2004, Cancer Res 64, 9209-9216), and by contrast the clinically localized tumors are generally androgen-dependent, it was evaluated whether there was an enrichment of androgen-regulated proteomic alterations discovered by the screen. Androgen regulated genes (ARGs) are essential for the normal development of the prostate as well as the pathogenesis of prostate cancer (Culig et al., 1998, Prostate 35, 63-70; Koivisto et al., 1998, Nat Med 4, 844-847; Mooradian et al., 1987, Endocr Rev 8, 1-28). Velasco et al. developed a meta-analysis of ARGs, which represents a cross-comparison of 4 gene expression (DePrimo et al., 2002, Genome Biol 3, RESEARCH0032; Nelson et al., 2002, Proc Natl Acad Sci U S A 99, 11890-11895; Segawa et al., 2002, Oncogene 21, 8749-8758; Velasco et al., 2004, Endocrinology 145, 3913-3924) and 2 SAGE datasets (Waghray et al., 2001, Proteomics 1, 1327-1338; Xu et al., 2001, Int J Cancer 92, 322-328). ARGs were then defined as a union of these 6 datasets, all of which represented functional induction of mRNA transcript by androgen in vitro. 27 out of the 150 protein alterations (exclusive of post-translational modifications) identified as being differential between metastatic and clinically localized disease were designated as androgen-regulated by the Velasco et al (Velasco et al., 2004, supra) ARG compendium.

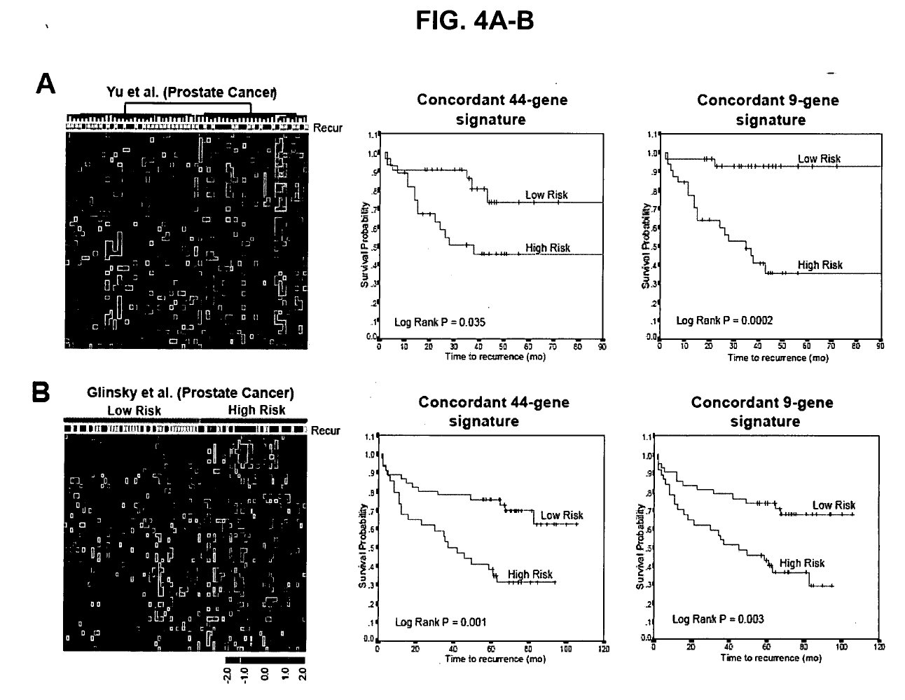

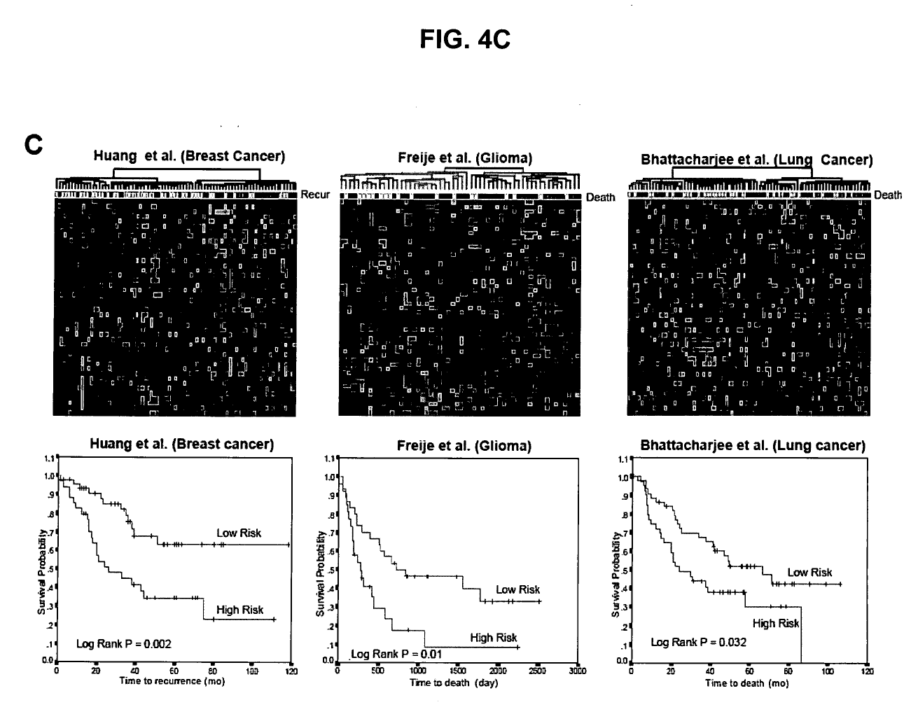

To demonstrate that this finding is statistically significant, random sets of 150 genes were selected from the Yu et al. (Yu et al., 2004, supra) or the Glinsky et al. (Glinsky et al., 2004, supra) prostate cancer profiling studies. It was found that the chance of selecting 27 ARGs was minimal (p<0.0001 for the Yu et al. and p<0.001 for Glinsky et al.). Thus, androgenregulated proteins are significantly enriched in the differential comparison between androgen-dependent and independent prostate cancer. Out of the 156 proteomic alterations identified between metastatic and localized prostate cancer, 50 were concordant with mRNA transcript and 90 were discordant with mRNA transcript (FIG. 3B, left panel). Many of these proteomic alterations were validated on individual tissue extracts to confirm the high-throughput immunoblot analysis (FIG. 3C). EZH2, a Polycomb group protein previously characterized as being over-expressed in aggressive prostate and breast cancer (Kleer et al., 2003, Proc Natl Acad Sci USA 100, 11606-11611; Varambally et al., 2002, supra) was one of the 50 proteins identified as being concordantly over-expressed in metastatic tissues at the mRNA and protein level (FIGS. 3B and 3C). As EZH2 was a member of this 50 gene concordant signature, it was hypothesized that proteomic alterations that distinguish metastatic prostate cancer from clinically localized disease could serve as a multiplex “lethal” signature of prostate cancer progression when applied to clinically localized disease (i.e., “more aggressive” genes would be expressed in progressive prostate cancer). Prostate cancer gene expression datasets that monitored over 85% of the genes in the concordant genomic/proteomic signature were identified that included biochemical recurrence information (time to PSA recurrence), as well as reported on a reasonable cohort of clinically localized specimens (n>50). The prostate cancer gene expression datasets that fulfilled these criteria were carried out by Yu et al. (Yu et al., 2004, supra) and Glinsky et al. (Glinsky et al., 2004, supra), both of which represent Affymetrix oligonucleotide datasets and each of which measured 44 out of the 50 genes in the concordant signature. Prediction models were built with the Yu et al. data set and the performance was tested on the Glinsky et al. data set. Utilizing an approach described earlier (Ramaswamy et al., 2003, Nat Genet 33, 49-54), unsupervised hierarchical clustering in the space of this 44-gene concordant signature resulted in two main clusters of individuals in the Yu et al. study (FIG. 4A). Kaplan-Meier (KM) survival analysis of the clusters indicated that the two groups of individuals are significantly different based on time to recurrence status (P=0.035, FIG. 4A). When the 90 discordant genes (mRNA transcripts that are not qualitatively concordant with protein levels) were used, it was found that these signatures did not generate a clinical outcome distinction (P=0.238). By permutation test, it was observed that random sets of 44 genes did not generate such prognostic distinctions, indicating that the concordant signature could not be achieved by chance. To assess the validity of this concordant 44-gene signature, the Glinsky et al. study was used as an independent test set (FIG. 4B). Each of the samples in the Glinsky dataset were classified as high- or low-risk based on a k-nearest neighbor (k-NN) model developed using the Yu et al. study as a training set (k=3). Based on the class predictions derived from the concordant signature, KM survival analysis revealed a significant difference in survival based on the risk stratification (P=0.001, FIG. 4B). This was not the case with the discordant signature when applied to the Glinsky et al. sample set (P=0.556). Multivariate Cox proportional-hazards regression analysis of the risk of recurrence was carried out on the Glinsky et al. validation set. Table 1 shows that the concordant signature predicted recurrence independently of the other clinical parameters such as surgical margin status, Gleason sum, and pre-operative PSA. With an overall hazard ratio of 3.66 (95% CI: 1.36-7.02, P<0.001), it was by far the strongest predictor of prostate cancer recurrence in the model.

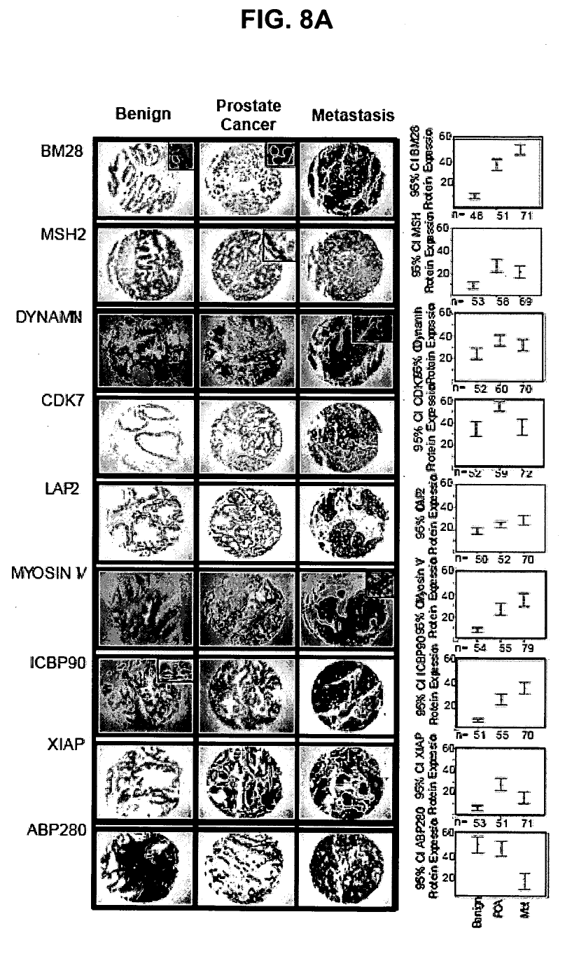

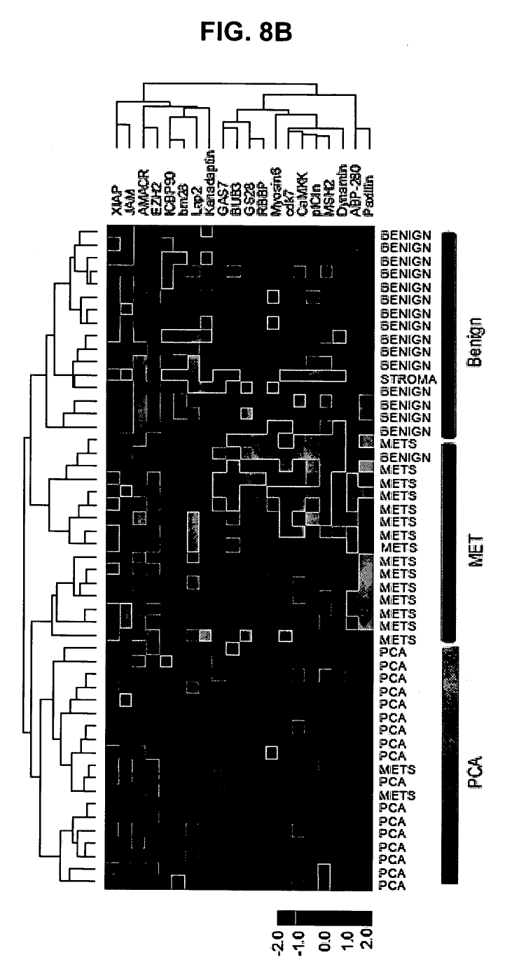

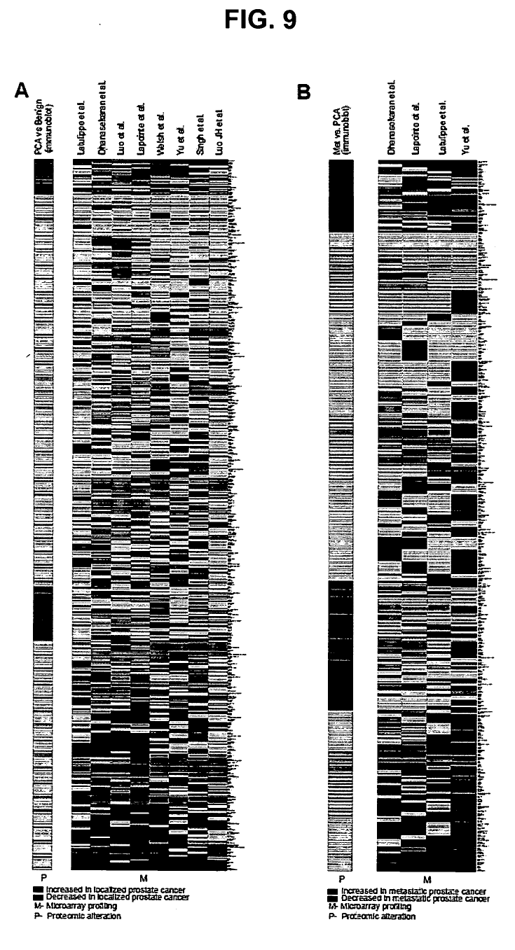

Next, the 44-gene concordant signature of prostate cancer progression was refined by reducing the number of genes required. By using the Yu et al. study as a training set, the 44 concordant genes were ranked by a univariate cox model. The same clustering procedure was employed to identify two clusters based on the top number of genes ranging from a minimum of 5 to a maximum of 44. Based on this iterative analysis, 9 genes were identified that demarcated two main clusters that differed most significantly by KM survival analysis (FIG. 4A). The Glinsky et al. study was again used as an independent validation set confirming that the 9-gene concordant signature identified two groups of individuals which differed significantly based on recurrence (FIG. 4B, FIG. 8). Together, this integrative analysis shows that MRNA transcripts that correlate with protein levels in metastatic prostate cancer can be used as gene predictors of progression in clinically localized disease.

Next, the generality of the larger 44-gene concordant signature of aggressiveness in other solid tumors was investigated. Four tumor profiling datasets from the Oncomine compendium (Rhodes et al., 2004, Neoplasia 6, 1-6) were identified that fulfilled the same criteria that were used in the prostate cancer analyses. In 95 primary breast adenocarcinomas (van 't Veer et al., 2002, Nature 415, 530-536), tumors bearing the 44-gene lethal proteomics signature were more likely to progress to metastasis than those lacking this signature (P=0.0025. A similar result was observed in 80 primary breast infiltrating ductal carcinomas (Huang et al., 2003, Lancet 361, 1590-1596) (P=0.002, FIG. 4C). This result was also observed in a series of 84 primary lung adenocarcinomas (Bhattacharjee et al., 2001, supra) (P=0.03; FIG. 4C) and 56 gliomas (Freije et al., 2004, Cancer Res 64, 6503-6510) (P=0.01; FIG. 4C). The smaller 9-gene model was only effective in discriminating prognostic classes in the glioma study (P=0.016) but not in the other solid tumors. This shows that the 9-gene model is specific for prostate cancer while the 44-gene model has more universal applicability. It should be understood that subsets of these groups also find use, as well as groups that add, subtract, or substitute one or more markers.

Taken together, the results of this example show that the lethal proteomic/genomic signature identified by the integrative analysis of metastatic prostate cancer has utility in the prognostication of clinically localized solid tumors in general. While these proteomic alterations can serve as a multiplex biomarker of cancer aggressiveness, they may also shed light into the biology of neoplastic progression. As proteins, rather than RNA transcripts, are the primary effectors of the cell, they play the central and most distal role in the functional pathways to cancer.

EZH2, which was previously have shown to have a role in prostate cancer progression (Varambally et al., 2002, supra), is a member of this concordant genomic/proteomics signature. For example, this screen identified Aurora-A kinase (STK15) as being overexpessed in metastatic prostate cancer as well as being a member of the 44-gene concordant signature. This serine-threonine kinase has been shown to be amplified in a number of human cancers (Jeng et al., 2004, Clin Cancer Res 10, 2065-2071; Neben et al., 2004, Cancer Res 64, 3103-3111), play a key role in G2/M cell cycle progression (Hirota et al., 2003, Cell 114, 585-598), and inhibit p53 (Katayama et al., 2004, Nat Genet 36, 55-62), among other functions. Another cancer regulatory molecule in the 44-gene concordant signature was KRIP1 (KAP-1), which is known to repress transcription via binding the methyltransferase SETDB1 (Schultz et al., 2002, Genes Dev 16, 919-932).

TABLE 1
Multivariable Proportional-Hazards Analysis of the Risk of
Recurrence as A First Event on the Glinsky et. al. Validation Set

| | Hazard Ratio | |
|---|---|---|
| Variable | (95% CI) | P Value |
| High-Risk signature (vs. | 3.66 (1.77-7.59) | <0.001 |
| low-risk signature) | | |
| PSA | 1.04 (1.00-1.09) | 0.043 |
| Gleason Sum Score | | |
| Score >7 (vs. score <=7) | 1.73 (0.79-3.76) | 0.17 |
| Tumor Stage | | |
| Stage T2 (vs. stage T1) | 0.85 (0.42-1.75) | 0.67 |
| Age | 1.06 (1.00-1.13) | 0.06 |
| Surgical Margins | | |
| Positive (vs. negative) | 2.18 (0.92-5.18) | 0.08 |

TABLE 2

| | | | Total | Number of samples | | |
|---|---|---|---|---|---|---|
| Authors | Journal | Array type | genes | Benign | Localized | Metastatic |
| Dhanasekaran, SM. | Nature, 412: 822 | cDNA | 9984 | 19 | 14 | 20 |
| et al. | | | | | | |
| Luo, J., et al. | Cancer | cDNA | 6500 | 9 | 16 | 0 |
| | Research, | | | | | |
| | 61: 4683 | | | | | |
| Lapointe, J., et | PNAS., | cDNA | 19124 | 41 | 61 | 9 |
| al. | 101(3): 811 | | | | | |
| Singh, D., et | Cancer Cell, | Affy HG- | 12626 | 50 | 52 | 0 |
| al. | 1(2): | U95Av2 | | | | |
| | 203, 2002 | | | | | |
| Welsh, JB., et | Cancer | Affy HG- | 12626 | 9 | 23 | 1 |
| al. | Research, | U95A | | | | |
| | 61: 5974 | | | | | |
| Latulippe, E., | Cancer | Affy HG-U95 | 62840 | 3 | 23 | 9 |
| et al. | Research, | | | | | |
| | 62: 4499 | | | | | |
| Luo, JH., et al. | Mol. Carcinog., | Affy HG- | 12626 | 15 | 15 | 0 |
| | 33(1): 25 | U95A | | | | |
| Yu, YP., et al. | J. Clin. Oncol., | Affy HG- | 37690 | 23 | 66 | 25 |
| | 22(14): 2790 | U95Av2, B, C | | | | |

TABLE 3

| Cancer | | | # of | |
|---|---|---|---|---|
| Type | Authors | Journal | genes* | Sample description |
| Prostate | Yu, YP., et | J. Clin. Oncol. | 44 | 21 patients had recurrence and 39 |
| | al. | 22(14): 2790 | | remained recurrence-free. |
| Prostate | Glinsky, | J. Clin. Invest. | 44 | 37 patients with recurrent and 42 |
| | GV., | 113(6): 913 | | patients |
| | et al. | | | with nonrecurrent disease |
| Glioma | Freije, WA., | Cancer Res. | 49 | 38 patients were dead and 18 were alive |
| | et al. | 64(18): 6503 | | |
| Breast | Huang, E et | Lancet. | 44 | 34 patients had recurrence and 46 |
| | al. | 361(9369): 1590 | | remained recurrence-free |
| Breast | Van't Veer, | Nature. | 48 | 45 patients advanced to metastasis and |
| | LJ. et. al. | 415(6871): 530 | | 51 samples haven't develop distant |
| | | | | metastases after 5 years |
| Lung | Bhattacharjee | PNAS. | 44 | 48 patients were dead and 36 were alive |
| | A et al. | 98(24): 13790 | | |

*Due to different microarray platforms, some genes were missed in particular studies.

TABLE 4
Androgen regulated genes among proteomic/genomic alterations between metastatic
prostate cancer and localized prostate cancer

| | | | | | | | | |
|---|---|---|---|---|---|---|---|---|
| | | | Segawa | Velasco | | Deprimo | | |
| | Protein | | et | et | Nelson et | et | | Androgen- |
| Unigene ID | Name | Gene Name | al. | al. | al | al. | Xu et al | Regulation* |

Concordant Genes

| | | | | | | | | |
|---|---|---|---|---|---|---|---|---|
| Hs.10842 | Ran | RAN | | | | | ✓ | + |
| Hs.134106 | Sek1 | MAP2K4 | ✓ | | | | | |
| Hs.154103 | Lim kinase | LIM | | | ✓ | | | + |
| Hs.157367 | Exportin | XPO1 | | | | | ✓ | + |
| Hs.171280 | ERAB | HADH2 | ✓ | | | | | |
| Hs.171952 | Occludin | OCLN | | | | ✓ | | − |
| Hs.171995 | PSA | KLK3 | ✓ | ✓ | ✓ | ✓ | | + |
| Hs.234521 | 3PK | MAPKAPK3 | ✓ | ✓ | | | | |
| Hs.236030 | BAF170 | SMARCC2 | ✓ | ✓ | | | | |
| Hs.256583 | DRBP76 | ILF3 | ✓ | | | | | + |
| Hs.298530 | RAB27 | RAB27A | ✓ | ✓ | | | | + |
| Hs.388677 | PAP | ACPP | | ✓ | | | | − |
| Hs.433612 | KRIP-1 | TRIM28 | | | | | ✓ | − |
| Hs.444118 | MCM6 | MCM6 | ✓ | | | | | |
| Hs.446336 | PAXILLIN | PXN | | ✓ | | | | − |

Discordant Genes

| | | | | | | | | |
|---|---|---|---|---|---|---|---|---|
| Hs.101174 | Tau-53 kD | MAPT | | ✓ | | | | − |
| Hs.15250 | PECI | PECI | | | | ✓ | | + |
| Hs.162089 | TPD52 | TPD52 | | | ✓ | ✓ | | + |
| Hs.167 | MAP2B | MAP2 | | ✓ | | | | |
| Hs.184298 | CDK7 | CDK7 | | ✓ | | | | + |
| Hs.324473 | ERK2 | MAPK1 | ✓ | | | | | − |
| Hs.406013 | Ms Cytokeratin | KRT18 | ✓ | | | ✓ | | − |
| Hs.408507 | TFII-I | GTF2I | | ✓ | | | | − |
| Hs.418004 | PTP I beta | PTPN1 | ✓ | | | | | + |
| Hs.511397 | MCAM | MCAM | ✓ | | | | | − |
| Hs.7557 | FKBP51 | FKBP5 | ✓ | ✓ | ✓ | | ✓ | + |
| Hs.79037 | HSP60 | HSPD1 | | ✓ | | | | + |

*“+” represents that the gene is androgen up-regulated; “−” represents that the gene is androgen down-regulated.
Significance were determined by student t-test on our published dataset (Dhanasekaran SM et al. FASEB J. 2005; 19(2): 243-5).

### Example 2

### Description of Selected Proteomic Alterations Identified by this Study

This Example describes functional taxonomy of the proteomic alterations characterized in Varambally et al, “Integrative Proteomic and Genomic Alterations of Prostate Cancer Progression.” Results are shown in Table 5.

TABLE 5

| | Localized | | Metastatic | | |
|---|---|---|---|---|---|
| Altered | Prostate Cancer | | Prostate Cancer | | |
| Proteins | RNA | Protein | RNA | Protein | Description |

Kinases and Phosphatases

| | | | | | |
|---|---|---|---|---|---|
| STK15 | + | U | + | + | Aurora-A is a centrosome-associated oncogenic |
| (Aurora-A) | | | | | kinase that has been implicated in the control of |
| | | | | | mitosis. Overexpression of Aurora-A has been |
| | | | | | shown |
| | | | | | to result in chromosomal aberration, genomic |
| | | | | | instability and tumorigenesis. This kinase is |
| | | | | | known to |
| | | | | | be amplified in a number of human cancers and |
| | | | | | tumor cell lines (Jeng et al., 2004) (Neben et al., |
| | | | | | 2004). Aurora-A regulates the p53 pathway by |
| | | | | | inducing increased degradation of p53, leading to |
| | | | | | aberrant checkpoint responses and facilitating |
| | | | | | oncogenic transformation of cells (Katayama et al., |
| | | | | | 2004) and has been shown to play a key role in |
| | | | | | G2/M |
| | | | | | progression (Hirota et al., 2003). |
| Glycogen | + | U | U | − | GSK3 beta is a key regulator of signaling |
| Synthase | | | | | pathways |
| kinase | | | | | that involves cellular responses to Wnt, receptor |
| (GSK) | | | | | tyrosine kinases, and G-protein-coupled receptors. |
| 3 beta | | | | | It |
| | | | | | also plays a central role in a wide range of cellular |
| | | | | | processes, such as glycogen metabolism, cell cycle |
| | | | | | regulation and proliferation. The activity of GSK3 |
| | | | | | beta is regulated by phosphorylation on |
| | | | | | tyrosine/serine residues (Kim and Kimmel, 2000) |
| | | | | | (Doble and Woodgett, 2003) (Patel et al., 2004). |
| | | | | | Studies have indicated that GSK-3 beta may |
| | | | | | function |
| | | | | | as a repressor of AR-mediated transactivation and |
| | | | | | cell growth (Mazor et al., 2004). It is a |
| | | | | | downstream |
| | | | | | substrate of PI3K/Akt and phosphorylation and |
| | | | | | inactivation of GSK-3 beta results in the nuclear |
| | | | | | accumulation of beta-catenin (Sharma et al., |
| | | | | | 2002). |
| | | | | | Beta-catenin is known to act as a coactivator of |
| | | | | | AR. |

Nuclear Proteins

| | | | | | |
|---|---|---|---|---|---|
| Heterochromatin | U | U | U | + | Recruitment of mammalian HP1 to a euchromatic |
| protein 1 | | | | | promoter was shown to establish a silenced state |
| (HP1) alpha | | | | | (Ayyanathan et al., 2003) |
| BRG1 | + | + | + | − | Brahma related group protein forms complexes |
| | | | | | with |
| | | | | | HP1 alpha to induce a silenced state (Nielsen et |
| | | | | | al., |
| | | | | | 2002). |
| KRIP1 | U | + | + | + | KRAB-A interacting protein 1 (KRIP-1) represses |
| | | | | | transcription via binding to methyl transferase |
| | | | | | SETDB1 (Schultz et al., 2002) |
| ICBP90 | U | U | + | + | ICBP90 binds to one of the inverted CCAAT |
| (Inverted | | | | | boxes of |
| CCAAT box | | | | | the topoisomerase II alpha (TopoII alpha) gene |
| | | | | | promoter (Hopfner et al., 2000) and has been |
| | | | | | shown to |
| Binding | | | | | be regulated by E2F-1 (Unoki et al., 2004) the |
| Protein of 90 kDa) | | | | | functional significance of which is yet to be |
| | | | | | understood. ICBP90 shares structural homology |
| | | | | | with |
| | | | | | several other proteins, including Np95, which is |
| | | | | | known to function as core histones specific E3 |
| | | | | | ubiquitin ligase (Citterio et al., 2004) |
| BUB3 | + | + | U | + | BUB3 is a part of a large multi-protein kinetochore |
| | | | | | complex believed to be a key component of the |
| | | | | | checkpoint regulatory pathway(Logarinho et al., |
| | | | | | 2004). |
| BM28, | U | + | + | + | BM28 along with other minichromosome |
| minichromosome | | | | | maintenance |
| maintenance | | | | | proteins play an essential role in initiation and |
| 2 (MCM2) | | | | | regulation of eukaryotic DNA replication (Eward |
| | | | | | et al., 2004). BM28 was previously reported to be |
| | | | | | dysregulated in malignant prostate glands (Meng |
| | | | | | et al., 2001). It has been proposed that BM28 |
| | | | | | expression is an independent predictor of disease- |
| | | | | | free survival after definitive local therapy and |
| | | | | | finds use as a |
| | | | | | molecular marker for clinical outcome in prostate |
| | | | | | cancer (Meng et al., 2001). |
| P16INK4a | U | + | + | + | P16 INK4a strongly binds to cdk4 and cdk6 to |
| (Cyclin- | | | | | inhibit their ability to interact with cyclin D. It was |
| dependent | | | | | shown to be upregulated in high-grade prostatic |
| | | | | | epithelial neoplasia (Henshall et al., 2001). |
| MSH2 | + | + | + | + | Loss of mismatch repair function leads to the |
| mutS (E. coli) | | | | | accumulation of errors that normally occur during |
| homolog 2 | | | | | DNA replication. Mismatch repair genes are |
| | | | | | involved |
| | | | | | in repairing these errors. MSH2 is shown to be |
| | | | | | upregulated in prostate cancer (Velasco et al., |
| | | | | | 2002) |

Enzymes

| | | | | | |
|---|---|---|---|---|---|
| MMP19 | − | + | − | − | Among various enzymes, characterized in cancer, |
| | | | | | proteases remain the best studied group for their |
| | | | | | role |
| | | | | | in promoting and facilitating the spread of |
| | | | | | malignancy. MMP19 has been shown to be |
| | | | | | downregulated during transformation and |
| | | | | | dedifferentiation of breast epithelia (Djonov et al., |
| | | | | | 2001) |
| MMP23 | U | − | − | − | See MMP19. |
| Cathepsin D | U | + | U | + | Cathepsin D, a carboxyl protease that has been |
| | | | | | implicated as an important factor in tumor cell |
| | | | | | invasion, is known to be upregulated in prostate |
| | | | | | cancer (Cherry et al., 1998). |
| Ubc9 | U | + | + | + | SUMOylation of proteins which involves the |
| | | | | | covalent |
| | | | | | attachment of a SUMO (small ubiquitin-related |
| | | | | | modifier) to substrate proteins is known to play a |
| | | | | | key |
| | | | | | role in protein targeting and/or stability(Lin et al., |
| | | | | | 2003) (Hilgarth et al., 2004). The SUMO-1- |
| | | | | | conjugating enzyme Ubc9 is known to interact |
| | | | | | with |
| | | | | | androgen receptor(Poukka et al., 1999; Poukka et |
| | | | | | al., |
| | | | | | 2000). (AR), a ligand-activated transcription |
| | | | | | factor, |
| | | | | | belonging to the steroid receptor superfamily and |
| | | | | | modulates its transcriptional regulatory |
| | | | | | activity(Callewaert et al., 2004). |

Structural proteins

| | | | | | |
|---|---|---|---|---|---|
| Paxillin | + | U | − | − | Paxillin is a LIM domain containing protein. It is |
| | | | | | known to regulate the androgen receptor activity |
| | | | | | by |
| | | | | | binding and targeting the androgen receptor to the |
| | | | | | nuclear matrix(Kasai et al., 2003). |
| LAP 2 beta | U | U | + | + | Lamina-associated polypeptide (LAP) 2 beta is |
| | | | | | known to function in initiation of DNA replication |
| Dematin | − | U | − | + | Demantin is a cytoskeletal protein that bundles |
| | | | | | actin filaments in a phosphorylation-dependent |
| | | | | | manner. It also interacts with Ras-guanine |
| | | | | | nucleotide exchange factor Ras-GRF2 and is |
| | | | | | implicated in modulation of mitogen-activated |
| | | | | | protein kinase pathways (Lutchman et al., 2002) |
| Dynamin | U | U | U | + | Dynamin is a cytosolic protein with a key role in |
| | | | | | clathrin-mediated endocytosis (Fournier et al., |
| | | | | | 2003) |
| ABP280 | − | − | − | − | ABP280, an actin-binding cytoskeletal protein |
| (filamin A) | | | | | promote orthogonal branching of actin filaments |
| | | | | | and link actin filaments to membrane |
| | | | | | glycoproteins (Loy et al., 2003). And It is known |
| | | | | | to anchor various transmembrane proteins to the |
| | | | | | actin cytoskeleton and serves as a scaffold for a |
| | | | | | wide range of cytoplasmic signaling proteins. Like |
| | | | | | Paxillin, ABP280 FLNa interferesd with AR |
| | | | | | interdomain interactions and |
| | | | | | competesd with the coactivator transcriptional |
| | | | | | intermediary factor 2 to specifically down-regulate |
| | | | | | AR function. Like Paxillin, ABP280 is also know |
| | | | | | to |
| | | | | | regulate the androgen receptor activity by |
| | | | | | intefering with the interdomain interactions (Loy |
| | | | | | et al., 2003). |
| Myosin VI | + | + | − | − | Myosin VI plays a role in E-cadherin mediated |
| | | | | | border cell migration(Geisbrecht and Montell, |
| | | | | | 2002). It has been recently shown that Myosin VI |
| | | | | | plays a major role in invasion in of ovarian cancer |
| | | | | | (Yoshida et al., 2004). |
| Stathmin | + | U | + | + | Stathmin also known as OP-18 is a cytosolic |
| | | | | | phosphoprotein proposed to act as a relay |
| | | | | | integrating diverse cell signaling pathways, |
| | | | | | notably during the control of cell growth and |
| | | | | | differentiation. It is one of the key regulators of |
| | | | | | cell division known for its ability to destabilize |
| | | | | | microtubules in a phosphorylation-dependent |
| | | | | | manner (Curmi et al., 1999). |
| JAM-1 | U | U | U | + | JAM-1 is localized at the tight junctions of |
| | | | | | epithelial and endothelial cells and is involved in |
| | | | | | the regulation of junctional integrity and |
| | | | | | permeability. They are glycoproteins characterized |
| | | | | | by two immunoglobulin folds (VH- and C2-type) |
| | | | | | in the extracellular domain. This protein was |
| | | | | | implicated in the regulation of tight junctions and |
| | | | | | leukocyte transmigration. This protein was |
| | | | | | implicated in the regulation of tight junctions and |
| | | | | | leukocyte transmigration (Mandell et al., 2004) |

Apoptotic regulators

| | | | | | |
|---|---|---|---|---|---|
| XIAP | + | + | U | + | XIAP(X chromosome-linked inhibitor of apoptosis |
| | | | | | protein) is a potent inhibitor of apoptosis. It is a |
| | | | | | downstream target of Akt. Phosphorylation of |
| | | | | | XIAP by Akt protects it from degradation resulting |
| | | | | | in increased cell survival. and undergoes |
| | | | | | phosphorylation and stabilization by protecting |
| | | | | | from ubiquitination and degradation (Dan et al., |
| | | | | | 2004) |
| AIF | − | + | U | + | Apoptosis inducing (AIF) is ubiquitously |
| | | | | | factor |
| | | | | | expressed protein involved in the induction of |
| | | | | | apoptosis. The AIF precursor is synthesized in the |
| | | | | | cytosol and is imported into mitochondria. AIF is |
| | | | | | known to translocates through the outer |
| | | | | | mitochondrial membrane to the cytosol and to the |
| | | | | | nucleus upon apoptosis inducing signals(Daugas et |
| | | | | | al., 2000). |
| BID | U | + | U | + | BID is a BH3-only member of the Bcl-2 family |
| | | | | | that regulates cell death at the level of |
| | | | | | mitochondrial membranes. It has been shown that |
| | | | | | BID plays a role in tumor suppression (Zinkel et |
| | | | | | al., 2003). |
| BIM | U | U | U | + | Bim is a pro-apoptotic protein and is known to |
| | | | | | cause degenerative disorders when overexpressed |
| | | | | | (Bouillet et al., 2001) |
| BAFF, B- | U | U | U | + | BAFF is critical regulator for survival of normal B |
| lymphocyte | | | | | cells. Increased BAFF expression has been shown |
| stimulator | | | | | in non-Hodgkin lymphoma, as tumors transform to |
| (BLyS) | | | | | a more aggressive phenotype (Novak et al., 2004). |

* “+” or “−” denotes increased or decreased expression in localized prostate cancer relative to benign prostate tissue or metastatic prostate cancer relative to localized prostate cancer. “U” denotes that the expression level is unchanged or needs to be further verified due to the inconsistency across gene expression profiling studies.

In order to understand the biological pathways at work in the altered genes/proteins, pathway enrichment was analyzed using ONCOMINE analyses tools (Rhodes et al., 2004). Such analyses of the concordant genes revealed that there was a disproportionate number of genes with conserved E2F1 binding sites in their promoters (n=7, odds ratio [OR]=23.8, P<0.0001), genes localized to chromatin (n=3, OR=0.0053, P=0.005), and genes involved in the cell cycle (n=4, OR=9.1, P=0.006). The down-regulated lethal signature had a disproportionate number of Zn-binding proteins (n=4, OR=109.0, P<0.0001), genes involved in proteolysis (n=4, OR=12.1, P=0.0005), and genes involved in signal transduction (n=6, OR=7.6, P=0.001). Similarly pathway analysis of the discordant signature for enrichment of particular processes revealed that the discordant genes/proteins included a disproportionate number proteins localized in the cytosol (odds ratio=8.9, p-value=5.7e-5) and proteins that function in the apoptosis pathway (odds ratio=6.9, p-value=1.3e-4).

### Example 3

### Further Tissue Microarray Analysis

In order to further confirm the proteomic alterations as well as to investigate any clinical significance and diagnostic values of the novel markers, high throughput tissue microarray analyses was performed. Staining was done on a fraction of the markers identified by highthroughput screening of prostate tissue lysates that had IHC-compatible antibodies. The immunostaining patterns varied greatly. Results for a select group of proteins are presented in FIGS. 2A and 8A. BM28 demonstrates nuclear expression in the basal cells of benign prostate glands (inset first panel) and both localized (inset middle) and metastatic prostate cancers. The staining intensity is usually moderate to strong when positive by immunohistochemistry. The percentage of epithelial cells staining positive for BM28 increases with tumor progression. The staining pattern is similar to that seen with Ki-67, the proliferation marker. MSH2 protein expression is nuclear and strongest in prostate cancer samples. However, as previously reported, there is variable expression in localized prostate cancer (Velasco et al., 2002, Endocrinology 145, 3913-3924).

This is the first study that demonstrates MSH2 expression in a subset of metastatic prostate tumors. Some of the metastatic tumors did not demonstrate MSH2 expression. These findings are not associated with germline mutations and therefore the biologic significance of these alterations is unknown.

Dynamin demonstrates a cytoplasmic and membranous expression pattern. Protein expression parallels prostate cancer progression. Expression is seen in benign prostate tissues but tends to be more diffuse and intense in localized tumors and metastatic tumors. The metastatic tumors demonstrate less membranous staining (inset right side) as compared to the localized tumors. CDK7 protein expression is seen in the nucleus of benign prostate, atrophy, localized prostate cancer, hormone sensitive and hormone refractory prostate cancer. The staining patterns can be generalized as follows. CDK7 shows the strongest and most uniform expression in clinically localized prostate cancer. The analysis does not quantify the total number of cells per unit area. Because tumor cells are more densely packed, one would expect that tissue extracts would demonstrate higher expression in localized prostate cancer and metastatic tumors. LAP2 demonstrates exclusively nuclear expression. The expression is seen in benign prostate tissue in the larger ductal structures and in basal cells. The strongest expression in the benign samples is in the ducts. Tumors demonstrate variable levels of nuclear expression. The association with prostate cancer progression may in part be due to the quantity of nuclei per unit area as opposed to significant differences in protein expression due to neoplastic transformation. Myosin VI demonstrates membranous and cytoplasmic protein expression. There is a trend towards higher expression with prostate cancer progression.

ICBP90 demonstrates intense nuclear expression that corresponds with tumor progression. ICBP90, when expressed, is moderate to strong. The extent of expression or percentage of cells increased in populations of tumor cells as compared to benign and atrophic prostate glands. The greatest expression was seen in hormone refractory tumors. Also, in the population of clinically localized tumors, ICBP90 expression was most extensive in tumors with a cribriform growth pattern (Gleason pattern 4), suggesting higher tumor grade. ILP/XIAP is expressed in the cytoplasm of neoplastic prostate epithelial cells and to a significantly lesser degree in the benign epithelial cells. Strong expression was seen in a few bony metastatic tumors. In general, the hormone naive metastatic tumors had lower expression as compared to the hormone refractory tumors.

CamKK demonstrates cytoplasmic and nuclear protein expression with a slight increase going from benign prostate tissue to metastatic prostate cancer. However, examples of high expression in benign and low expression in metastatic samples could be found.

JAM1 demonstrates membranous expression, seen strongest and most consistently in hormone refractory prostate cancer. Hormone naive metastatic prostate cancer has weak protein expression. Expression can also be seen in benign prostate tissue and localized prostate cancer. The expression of JAM1 may also be affected by the number of epithelial cells per unit area. PICIn demonstrates both nuclear and cytoplasmic protein expression. The nuclear expression is weak to moderate in benign prostate tissue and can also be seen in neoplastic tissues. The cytoplasmic protein expression increases with prostate cancer progression. The highest cytoplasmic expression is seen in metastatic PCA. A significant subset of metastatic tumors did not show strong expression. Co-chaperone protein p23 expression is predominantly cytoplasmic with some nuclear staining detected in some cases but always with cytoplasmic expression. Overall protein expression appears most consistently high in localized prostate cancer. In general, weak expression was seen in benign prostate tissue. Localized prostate cancer had more diffuse moderate to strong cytoplasmic staining. Metastatic tumors demonstrated strong protein expression as often as having no detectable protein expression.

The changes in the staining intensity are depicted in FIG. 2A and FIG. 8. Most of the cancer and metastatic tissues show higher staining intensity. The tissue microarray staining of 20 of the markers were analyzed by unsupervised clustering. Clustering of tissue microarrays has been reported earlier (Nielsen et al., 2003, supra). Similar to gene expression analyses (Eisen et al., 1998, supra; Perou et al., 2000, supra), unsupervised hierarchical clustering of the data revealed that the in situ protein levels could be used to classify prostate samples as benign, clinically localized prostate cancer, or metastatic disease (FIG. 8B). The greatest overall increase in expression is seen in the clinically localized tumors. Most of the metastatic samples and prostate cancer tissues cluster together as can be seen in FIG. 8B.

Additional markers validated by tissue microarray include ABP280, AMACR, BM28, CamKK, CDK7, Dynamin, EZH2, GS28, ICBP90, JAM1, Kanadaptin, LAP2, MSH2, Myosin VI, PAXILLIN, pICIn, RBBP, XIAP, BUB3, and GAS7.

Additional markers validated by immunoblot include, CAMKK, CASPASE 3, CASPASE 7, CATHEPSIN D, CDK7, C-FLIP, cIAP1, CO-chaperone protein p23, CPKC, CRP1, DcR1, DEMATIN, DR3, DRBP76, DYNAMIN, E2F3, ECA39, ERAB, EXPORTIN, EZH2, GAS7, GS28, GSK3-BETA, HP1 ALPHA, ICBP90, IGFBP2, ILK, INTEGRIN 5ALPHA, IRAK, JAM1, KRIP, LAP2, LIM-KINASE, MCAM, MLCK, MMP-19, MMP-23, MSH2, MYOSIN VI, NEXILIIN, NTF2, NUCLEOPORIN P62, P16INK4A, P67phox, PAXILLIN, PCNA, PICIN, P-MAPK, p-PKR, PRO-CASPASE7, PSA, PTP1-BETA, PTP1C, RAB27, RACK1, RAL A, RBBP, S6K, SAPK/JNK, SHC HOMOLOG, SPROUTY4, Stathmin/OP18, TGF alpha, TROY, TRYROSINASE, UBc9, Vti1B, and XIAP.

Table 6 shows genes with altered expression in benign versus prostate cancer 5 identified using the proteomics analysis methods of the present invention. A (+) in the Blot column indicates proteins that are upregulated in prostate cancer relative to benign prostate, while a (−) indicates proteins that are down regulated.

TABLE 6

| | | | |
|---|---|---|---|
| | | Gene | |
| UG_ClusterID | Protein Name | Symbol | Blot |

Benign Vs Prostate Cancer

| | | | |
|---|---|---|---|
| Hs.118483 | Myosin VI | MYO6 | + |
| Hs.49598 | AMACR | AMACR | + |
| Hs.79037 | HSP60 | HSPD1 | + |
| Hs.184298 | CDK7 | CDK7 | + |
| Hs.162089 | TPD52 | TPD52 | + |
| Hs.78202 | BRG1 | SMARCA4 | + |
| Hs.418533 | BUB3 | BUB3 | + |
| Hs.171995 | PSA | KLK3 | + |
| Hs.440394 | MSH2 | MSH2 | + |
| Hs.124436 | GS28 | GOSR1 | + |
| Hs.417369 | plCln | CLNS1A | + |
| Hs.250822 | Aurora kinase A | STK6 | + |
| Hs.16003 | RBBP | RBBP4 | + |
| Hs.318381 | CK1 | CSNK1A1 | + |
| Hs.171280 | ERAB | HADH2 | + |
| Hs.356076 | XIAP | BIRC4 | + |
| Hs.380403 | BMI-1 | PCGF4 | + |
| Hs.437508 | ACT1 | C6orf4 | + |
| Hs.78996 | PCNA | PONA | + |
| Hs.250882 | B2 Bradykinin receptor | BDKRB2 | + |
| Hs.349611 | PKC alpha | PRKCA | + |
| Hs.76364 | AIF | AIF1 | + |
| Hs.421349 | p16INK4A | CDKN2A | + |
| Hs.54433 | Janusin | TNR | + |
| Hs.290270 | GKAP | DLGAP1 | + |
| Hs.98493 | XRCC | XRCC1 | + |
| Hs.348446 | SAPK/JNK2 | MAPK9 | + |
| Hs.141125 | Casp 3 | CASP3 | + |
| Hs.134106 | Sek1 | MAP2K4 | + |
| Hs.300825 | BID | BID | + |
| Hs.121575 | Cathepsin D-28 kD | CTSD | + |
| Hs.172865 | CSTF50 | CSTF1 | + |
| Hs.236030 | BAF170 | SMARCC2 | + |
| Hs.154057 | MMP19 | MMP19 | + |
| Hs.75360 | Carboxypeptidase E | CPE | + |
| Hs.57101 | BM28 | MCM2 | + |
| Hs.2007 | FAS ligand | TNFSF6 | + |
| Hs.302903 | Ubc9 | UBE2I | + |
| Hs.355693 | co-chaperone protein p23 | TEBP | + |
| Hs.433612 | KRIP-1 | TRIM28 | + |
| Hs.256583 | DRBP76 | ILF3 | + |
| Hs.1189 | E2F3 | E2F3 | + |
| Hs.9216 | Casp7 | CASP7 | + |
| Hs.82116 | MYD88 | MYD88 | − |
| Hs.484782 | DFF45 | DFFA | − |
| Hs.388677 | PAP | ACPP | − |
| Hs.298530 | RAB27 | RAB27A | − |
| Hs.324473 | ERK2 | MAPK1 | − |
| Hs.241431 | G alpha t | GNAO1 | − |
| Hs.211819 | MMP23 | MMP23B | − |
| Hs.42806 | Cab45 | Cab45 | − |
| Hs.433611 | PDK1 | PDK1 | − |
| Hs.226133 | GAS 7 | GAS7 | − |
| Hs.437191 | PTRF | PTRF | − |
| Hs.408754 | EB1 | MAPRE1 | − |
| Hs.149609 | Integrin 5 alpha | ITGA5 | − |
| Hs.6241 | PI3 Kinase | PIK3R1 | − |
| Hs.390616 | PAK3 | PAK3 | − |
| Hs.195464 | ABP280 | FLNA | − |
| Hs.511397 | MCAM | MCAM | − |
| Hs.334174 | TROY | TNFRSF19 | − |
| Hs.437191 | PTRF | PTRF | − |
| Hs.408754 | EB1 | MAPRE1 | − |
| Hs.149609 | Integrin 5 alpha | ITGA5 | − |
| Hs.6241 | PI3 Kinase | PIK3R1 | − |
| Hs.390616 | PAK3 | PAK3 | − |
| Hs.195464 | ABP280 | FLNA | − |
| Hs.511397 | MCAM | MCAM | − |
| Hs.334174 | TROY | TNFRSF19 | − |

Table 7 shows proteins with altered expression in metastatic prostate cancer vs. local prostate cancer identified using the proteomics analysis methods of the present invention. A (+) in the Blot column indicates proteins that are upregulated in metastatic prostate cancer relative to local prostate cancer, while a (−) indicates proteins that are down regulated.

TABLE 7

| | | | |
|---|---|---|---|
| | | Gene | |
| UG_ClusterID | Protein Name | Symbol | Blot |

Prostate Cancer Vs Metastatic tumors

| | | | |
|---|---|---|---|
| Hs.250822 | Aurora kinase A | STK6 | + |
| Hs.444082 | EZH2 | EZH2 | + |
| Hs.528342 | Nucleoporin p62 | NUP62 | + |
| Hs.11355 | LAP2 | TMPO | + |
| Hs.6906 | Ral A | RALA | + |
| Hs.302903 | Ubc9 | UBE2I | + |
| Hs.157367 | Exportin | XPO1 | + |
| Hs.421349 | p16INK4A | CDKN2A | + |
| Hs.440394 | MSH2 | MSH2 | + |
| Hs.419995 | Vti1B | VTI1B | + |
| Hs.511739 | Uba2 | UBA2 | + |
| Hs.236030 | BAF170 | SMARCC2 | + |
| Hs.433612 | KRIP-1 | TRIM28 | + |
| Hs.171952 | Occludin | OCLN | + |
| Hs.444118 | MCM6 | MCM6 | + |
| Hs.10842 | Ran | RAN | + |
| Hs.348446 | SAPK/JNK2 | MAPK9 | + |
| Hs.256583 | DRBP76 | ILF3 | + |
| Hs.77793 | Csk | CSK | + |
| Hs.171280 | ERAB | HADH2 | + |
| Hs.209983 | Stathmin/Metablastin | STMN1 | + |
| Hs.108106 | ICBP90 | UHRF1 | + |
| Hs.159557 | Karyopherin alpha 2 | KPNA2 | + |
| Hs.95577 | Cdk4 | CDK4 | + |
| Hs.57101 | BM28 | MCM2 | + |
| Hs.184298 | CDK7 | CDK7 | + |
| Hs.89499 | 5-Lipoxygenase | ALOX5 | + |
| Hs.250882 | B2 Bradykinin receptor | BDKRB2 | + |
| Hs.258538 | Striatin-119 kD | STRN | + |
| Hs.155560 | Calnexin | CANX | + |
| Hs.23103 | Bet1 | BET1 | + |
| Hs.254321 | alpha-Catenin | CTNNA1 | + |
| Hs.166011 | pp120-102 kD | CTNND1 | + |
| Hs.437495 | PI31 | PSMF1 | + |
| Hs.171626 | p19Skp 1 | SKP1A | + |
| Hs.380403 | BMI-1 | PCGF4 | + |
| Hs.282346 | TOPO2 beta | TOP2B | + |
| Hs.152978 | Psme3-29 kD | PSME3 | + |
| Hs.300825 | BID | BID | + |
| Hs.418004 | PTP I beta | PTPN1 | + |
| Hs.5662 | RACK1 | GNB2L1 | + |
| Hs.76930 | alpha-Synuclein | SNCA | + |
| Hs.409546 | p190 | ARHGAP5 | + |
| Hs.437475 | Stat6 | STAT6 | + |
| Hs.356076 | XIAP | BIRC4 | + |
| Hs.506845 | JAM-1 | F11R | + |
| Hs.5215 | EIF-6 | ITGB4BP | + |
| Hs.121575 | Cathepsin D-28 kD | CTSD | + |
| Hs.305890 | BCL-x | BCL2L1 | + |
| Hs.270737 | BAFF | TNFSF13B | + |
| Hs.462864 | Annexin II-34 kD | ANXA2 | + |
| Hs.417369 | plCln | CLNS1A | + |
| Hs.441202 | GFRalpha2 | GFRA2 | + |
| Hs.356630 | NTF2 | NUTF2 | + |
| Hs.512638 | TIP120 | TIP120A | + |
| Hs.355861 | Nmt55 | NONO | + |
| Hs.271225 | FACTp140 | SUPT16H | + |
| Hs.2053 | Tyrosinase | TYR | + |
| Hs.141125 | Casp 3 | CASP3 | + |
| Hs.389182 | HP1 alpha | CBX5 | + |
| Hs.110713 | DEK | DEK | + |
| Hs.418533 | BUB3 | BUB3 | + |
| Hs.73722 | Ref1 | APEX1 | + |
| Hs.78996 | PCNA | PCNA | + |
| Hs.123044 | NHE-3 | SLC9A3 | + |
| Hs.170009 | TGFalpha | TGFA | + |
| Hs.54433 | Janusin | TNR | + |
| Hs.380938 | Syntaxin 8 | STX8 | + |
| Hs.156637 | c-Cbl | CBLC | + |
| Hs.949 | p67PHOX | NCF2 | + |
| Hs.433326 | IGFBP2 | IGFBP2 | + |
| Hs.119684 | DcR1 | TNFRSF10C | + |
| Hs.436132 | Dynamin | DNM1 | + |
| Hs.84063 | BIM/BOD | BCL2L11 | + |
| Hs.274122 | Dematin | EPB49 | + |
| Hs.101174 | Tau-53 kD | MAPT | + |
| Hs.15250 | PECI | PECI | + |
| Hs.394609 | Neurotensin Receptor 3-117 kD | SORT1 | + |
| Hs.512640 | 80K-H | PRKCSH | + |
| Hs.438993 | ECA39 | BCAT1 | + |
| Hs.7557 | FKBP51 | FKBP5 | − |
| Hs.390616 | PAK3 | PAK3 | − |
| Hs.406013 | Ms Cytokeratin | KRT18 | − |
| Hs.75360 | Carboxypeptidase E | CPE | − |
| Hs.98493 | XRCC | XRCC1 | − |
| Hs.306000 | Kanadaptin | SLC4A1AP | − |
| Hs.408507 | TFII-I | GTF2I | − |
| Hs.374638 | FKBP12 | FKBP1A | − |
| Hs.172865 | CSTF50 | CSTF1 | − |
| Hs.324473 | ERK2 | MAPK1 | − |
| Hs.154057 | MMP19 | MMP19 | − |
| Hs.82116 | MYD88 | MYD88 | − |
| Hs.76111 | Beta-Dystroglycan | DAG1 | − |
| Hs.226133 | GAS 7 | GAS7 | − |
| Hs.162089 | TPD52 | TPD52 | − |
| Hs.433795 | SHC transforming protein 1 | SHC1 | − |
| Hs.182018 | IRAK | IRAK1 | − |
| Hs.167 | MAP2B | MAP2 | − |
| Hs.243491 | Casp8 | CASP8 | − |
| Hs.1189 | E2F3 | E2F3 | − |
| Hs.79037 | HSP60 | HSPD1 | − |
| Hs.241431 | G alpha t | GNAO1 | − |
| Hs.511397 | MCAM | MCAM | − |
| Hs.433611 | PDK1 | PDK1 | − |
| Hs.78202 | BRG1 | SMARCA4 | − |
| Hs.1030 | RIN1 | RIN1 | − |
| Hs.16003 | RBBP | RBBP4 | − |
| Hs.349611 | PKC alpha | PRKCA | − |
| Hs.124436 | GS28 | GOSR1 | − |
| Hs.299558 | DR3 | TNFRSF25 | − |
| Hs.282359 | GSK3 beta | GSK3B | − |
| Hs.335786 | TIAR | TIAL1 | − |
| Hs.79748 | 4F2 hc/CD98HC | SLC3A2 | − |
| Hs.86858 | S6K | RPS6KB1 | − |
| Hs.268177 | Phospholipase C gamma 1 | PLCG1 | − |
| Hs.298530 | RAB27 | RAB27A | − |
| Hs.22370 | Nexilin | NEXN | − |
| Hs.171995 | PSA | KLK3 | − |
| Hs.388677 | PAP | ACPP | − |
| Hs.75799 | Prostasin | PRSS8 | − |
| Hs.386078 | Myosin light chain kinase(MLCK) | MYLK | − |
| Hs.134106 | Sek1 | MAP2K4 | − |
| Hs.437508 | ACT1 | C6orf4 | − |
| Hs.377908 | MYPT1 | PPP1R12A | − |
| Hs.154103 | Lim kinase | LIM | − |
| Hs.108080 | CRP1 | CSRP1 | − |
| Hs.149609 | Integrin 5 alpha | ITGA5 | − |
| Hs.195464 | ABP280 | FLNA | − |
| Hs.9216 | Casp7 | CASP7 | − |
| Hs.211819 | MMP23 | MMP23B | − |
| Hs.1288 | MSActin | ACTA1 | − |
| Hs.290270 | GKAP | DLGAP1 | − |
| Hs.234521 | 3PK | MAPKAPK3 | − |
| Hs.289107 | CIAP | BIRC2 | − |
| Hs.139851 | Caveolin 2-20 kD | CAV2 | − |
| Hs.25511 | Hic-5 | TGFB1I1 | − |
| Hs.446336 | PAXILLIN | PXN | − |
| Hs.49598 | AMACR | AMACR | − |
| Hs.408767 | alphaB Crystallin | CRYAB | − |
| Hs.355724 | c-FLIP | CFLAR | − |

Table 8 shows cell compartments and statistical analyses of 20 proteins used for tissue microarray analysis. One-way ANOVA with post hoc tests was conducted for each protein. Population variances were examined by Levene test. Tukey's HSD test was used to control the Type I error rate if the population variances were equivalent, otherwise, the Games-Howell procedure was used.

TABLE 8

| | | | | | | | | Corcondant Analysis | |
|---|---|---|---|---|---|---|---|---|---|
| | | | | | | | | with the | |
| | | Multiple Comparisons1 | | | | | | transcript expression | |
| Cell | | Benign vs. PCA Benign | | | | | | | PCA vs. |
| Protein | Compartment | vs. Met MET PCA | | | | | vs. | Benign PCA vs. | MET |
| DYNAMIN | Epithelium | | | | | | | ✓ | ✓ |
| AMACR | Epithelium | * | | * | | | | ✓ | |
| CDK7 | Epithelium | * | | | | * | | ✓ | ✓ |
| PICIN | Epithelium | | * | | | | * | ✓ | |
| XIAP | Epithelium | | * | | | | | ✓ | ✓ |
| MSH2 | Epithelium | | | | | | | | |
| CAMKK | Epithelium | | * | | | | * | | |
| BUB3 | Epithelium | | | | | | | | ✓ |
| GS28 | Epithelium | | | | | | | ✓ | ✓ |
| RBBP | Epithelium | | * | | | | * | ✓ | |
| GAS7 | Epithelium | | | | | | | | ✓ |
| EZH2 | Epithelium | | * | | * | | | | |
| ICBP90 | Epithelium | | * | | * | | | | |
| JAM | Epithelium | | | | | | | ✓ | ✓ |
| LAP2 | Epithelium | | | | | | | ✓ | |
| BM28 | Epithelium | | * | | * | | | ✓ | |
| MYOSIN6 | Epithelium | | * | | * | | | ✓ | ✓ |
| KANADAPTIN | Epithelium | | * | | | | | | |
| ABP-280 | Stroma | | | | * | | * | | ✓ |
| PAXILIN | Stroma | | | | * | | * | ✓ | ✓ |

1Benign: benign prostate tissue; PCA: clinically localized prostate cancer; MET: metastatic prostate cancer. One-way ANOVA with post hoc tests was conducted for each protein. Population variances were examined by Levene test. The Tukey's HSD test was used to control the Type I error rate if the population variances were equivalent, otherwise, the Games-Howell procedure was used instead. The analysis was carried out in SPSS 11.5.
*: The protein is significant at 0.05 level;
✓: The IHC result of the protein is concordant with its transcript expressions when examined by same procedure used in the integrative molecular analysis.

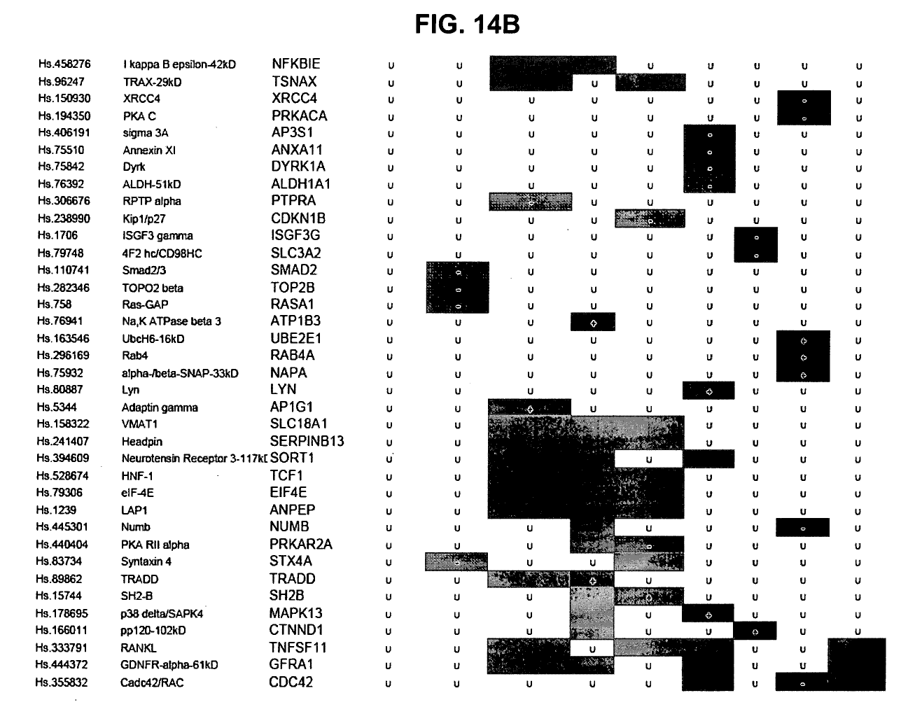

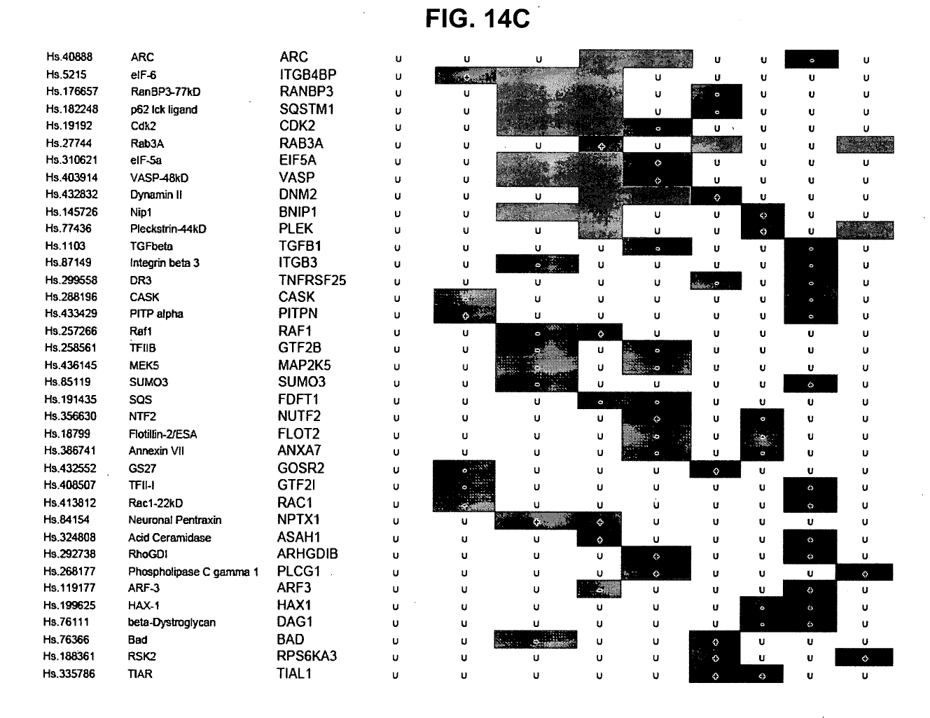

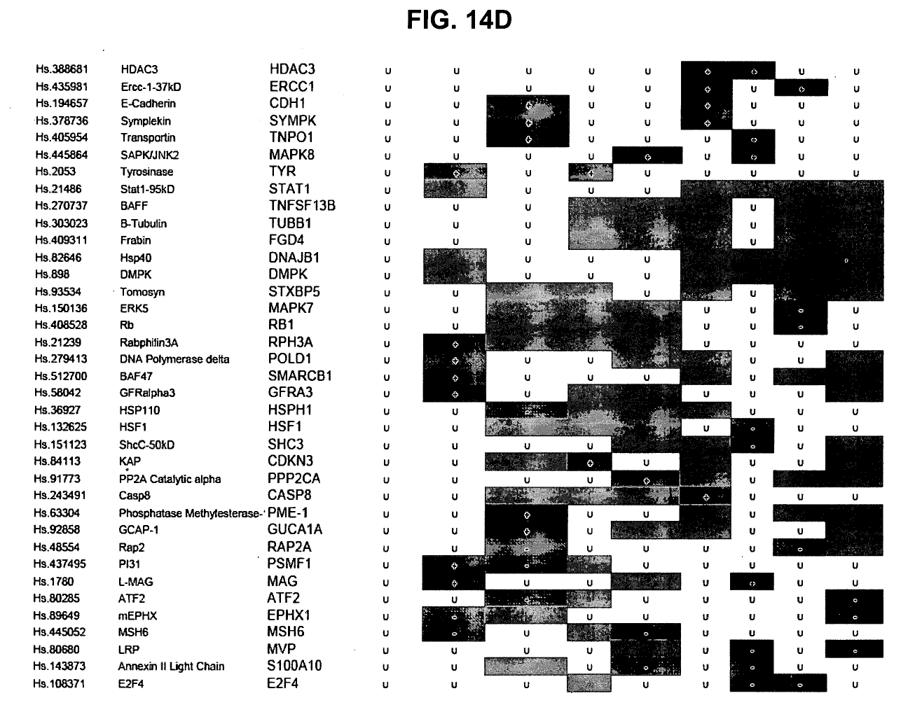

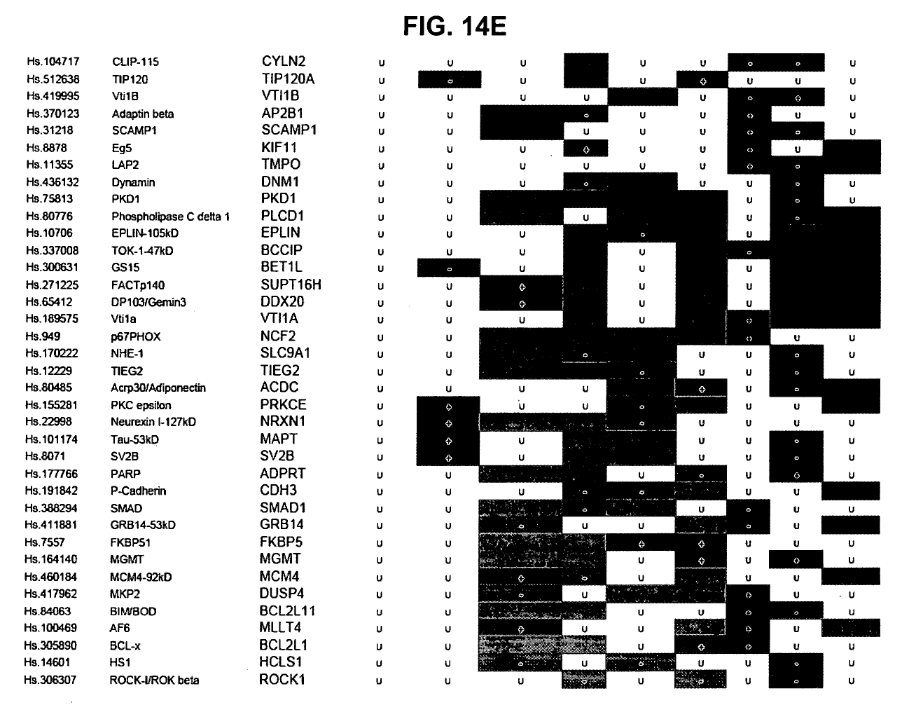

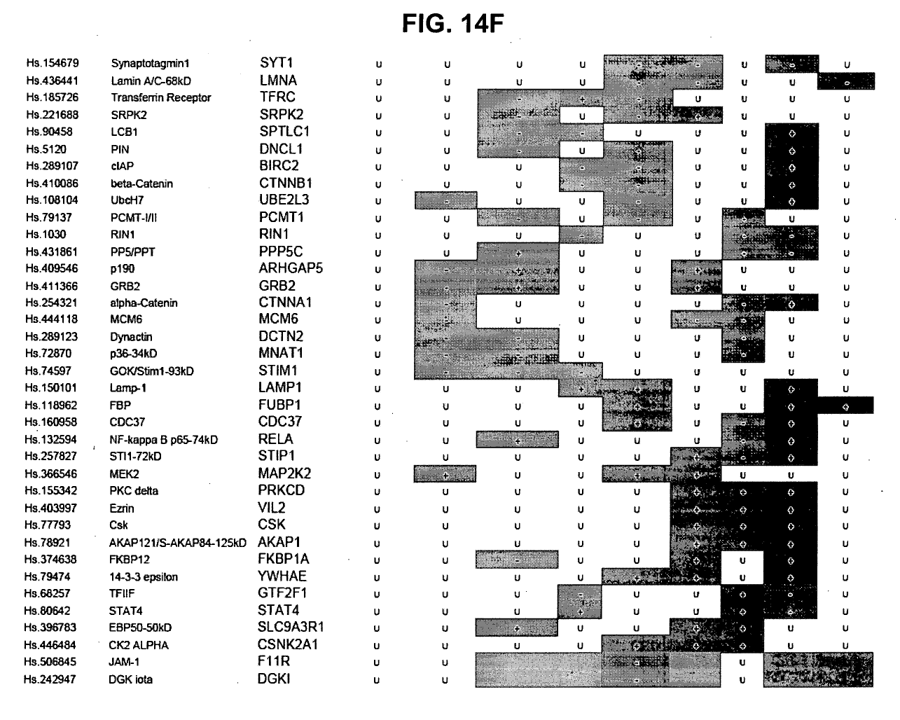

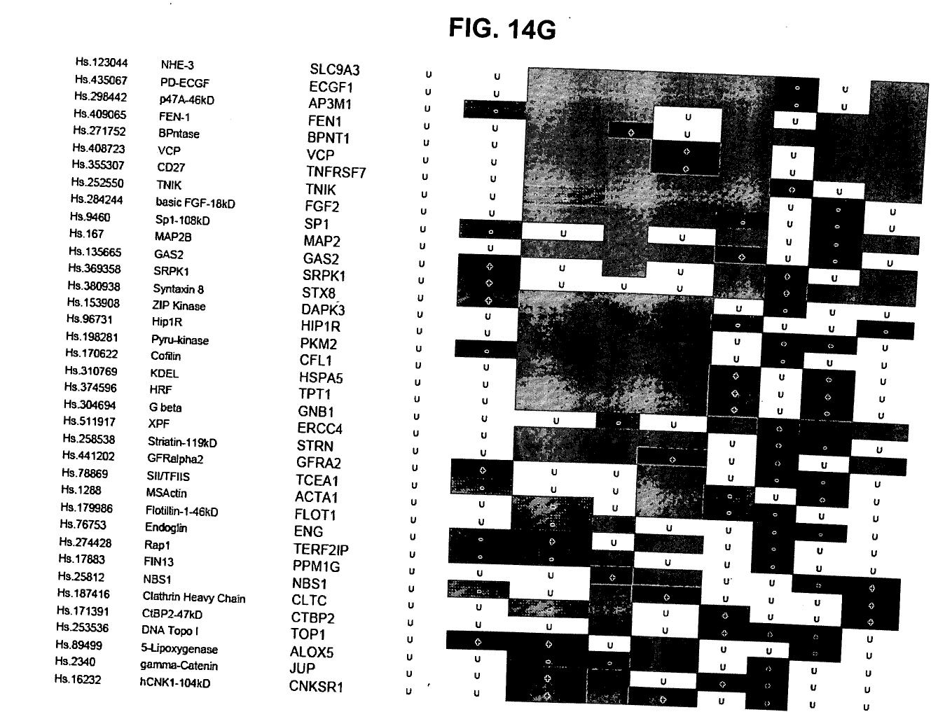

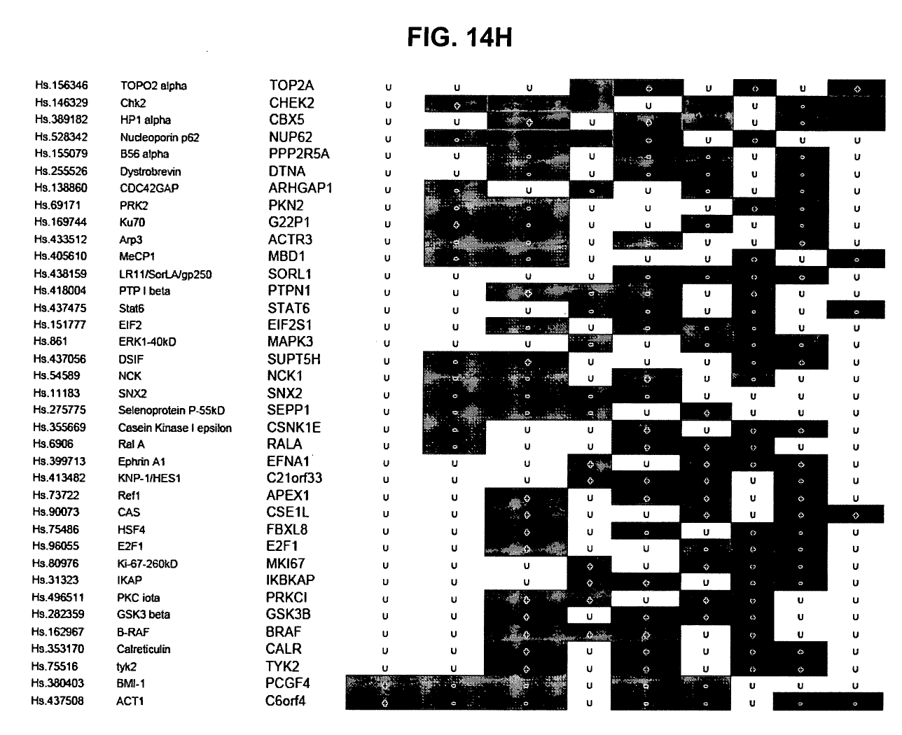

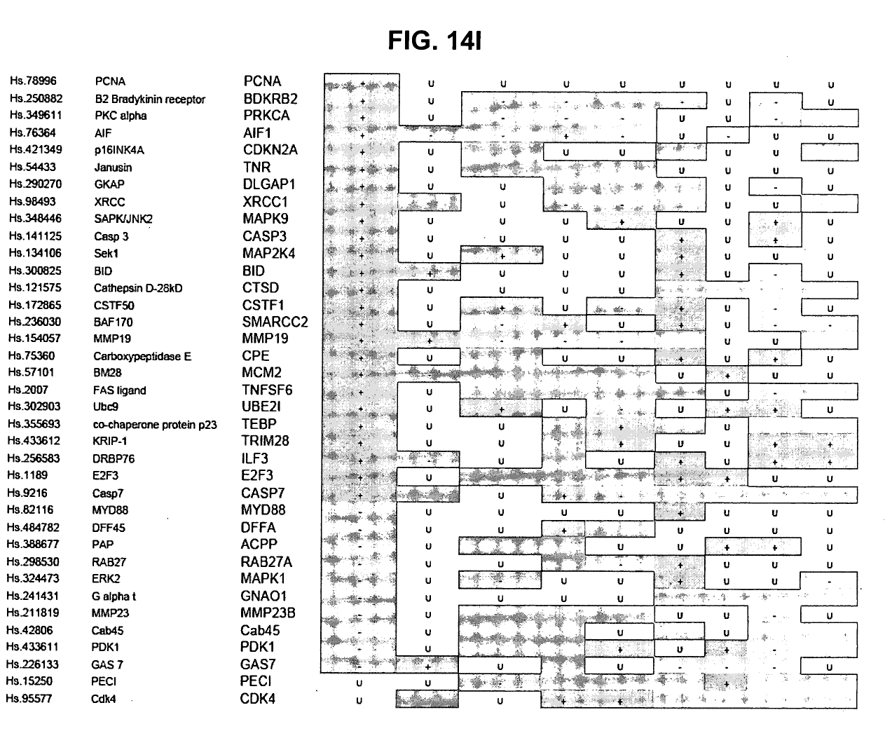

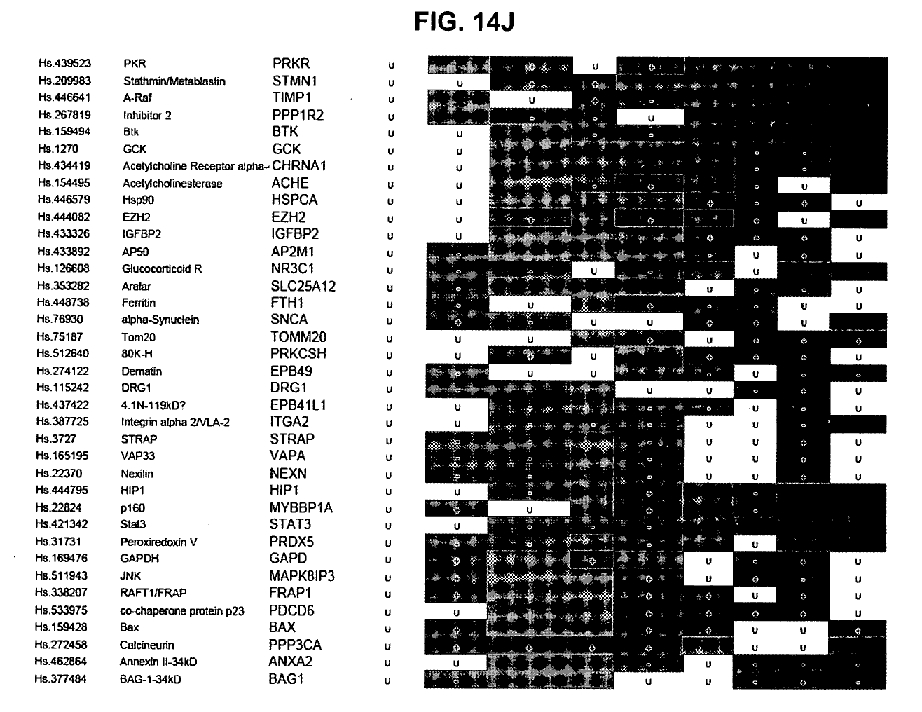

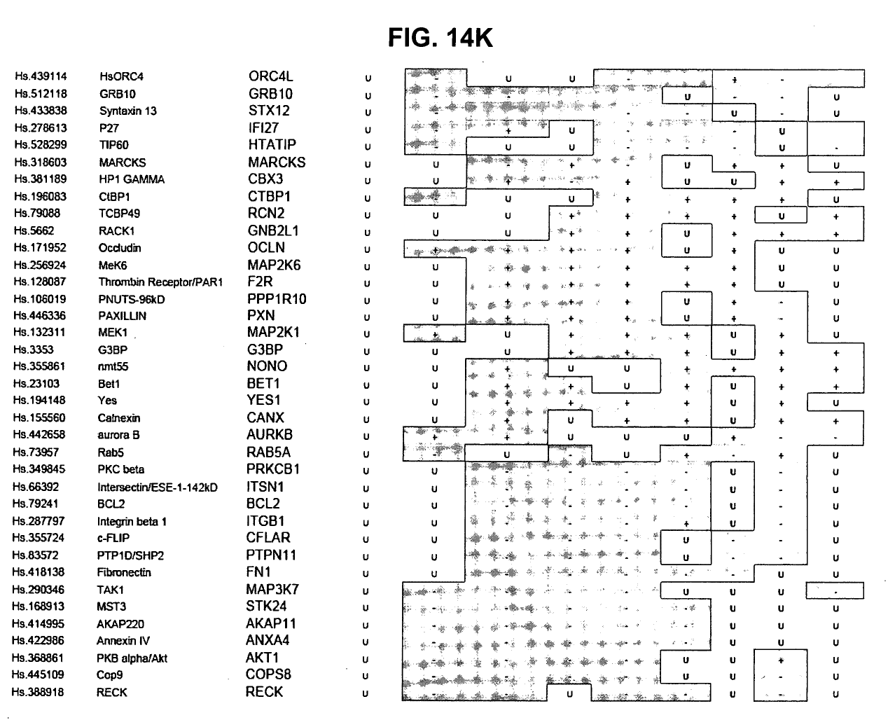

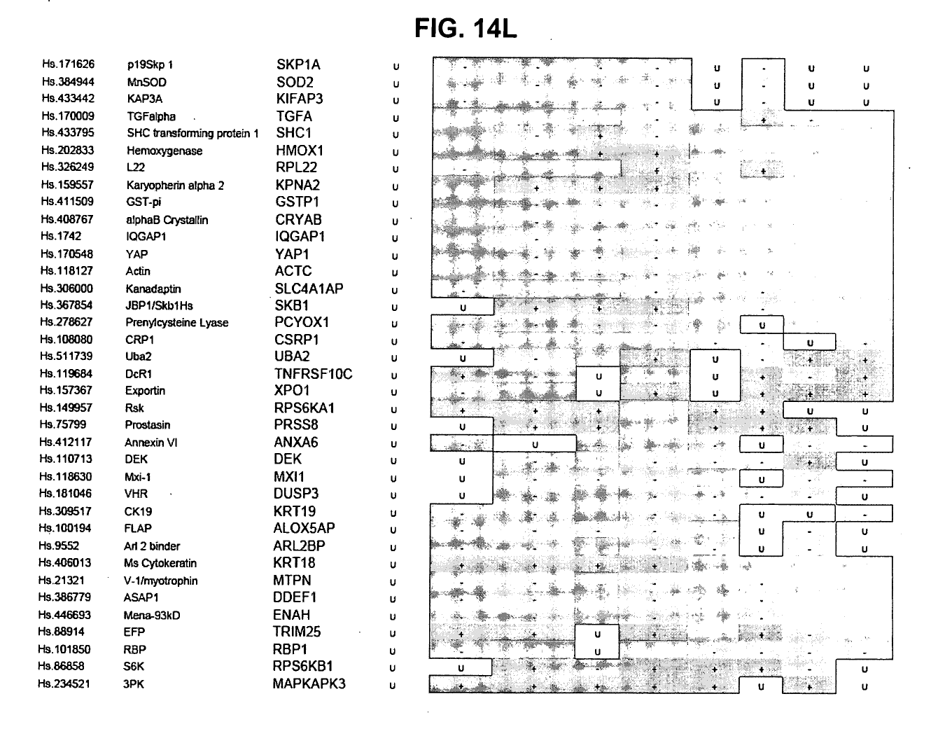

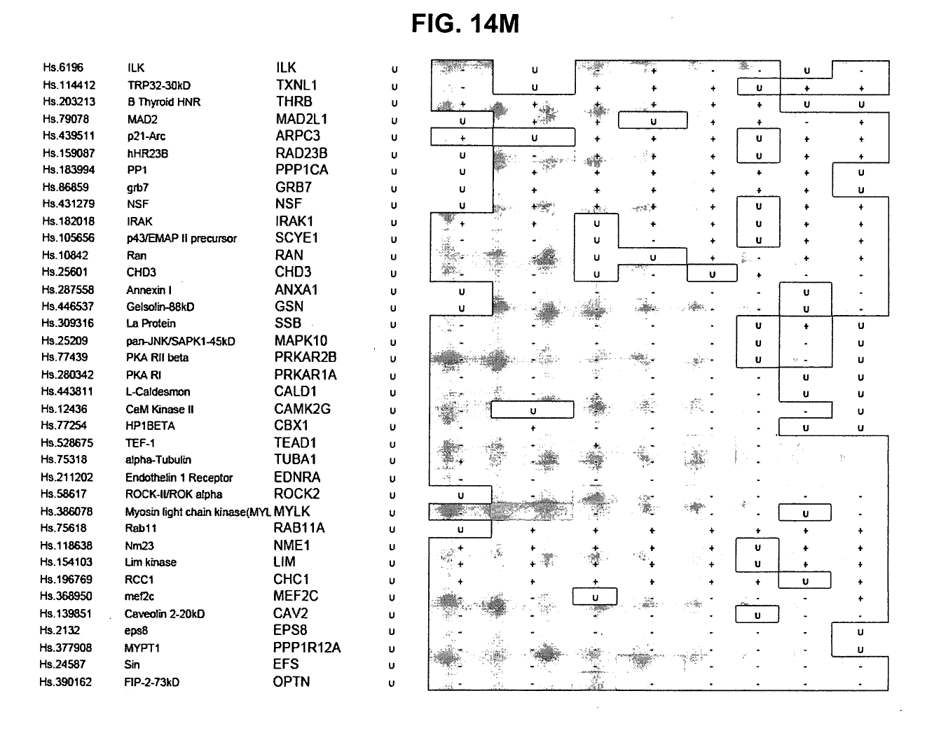

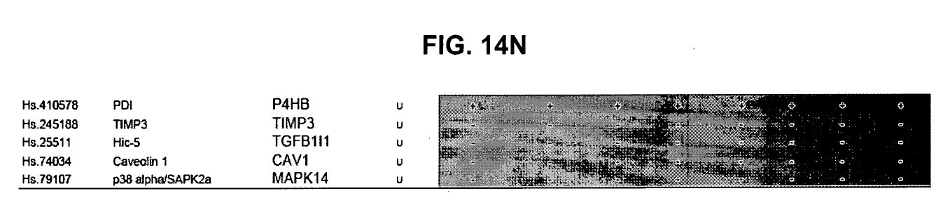

Table 9 (FIG. 14) shows proteomics alterations mapped to gene expression in clinically localized prostate cancer relative to benign prostate tissue. * “+”, “U”, or “−” denotes that the corresponding protein is increased, unchanged, or decreased respectively in clinically localized prostate cancer relative to benign prostate.

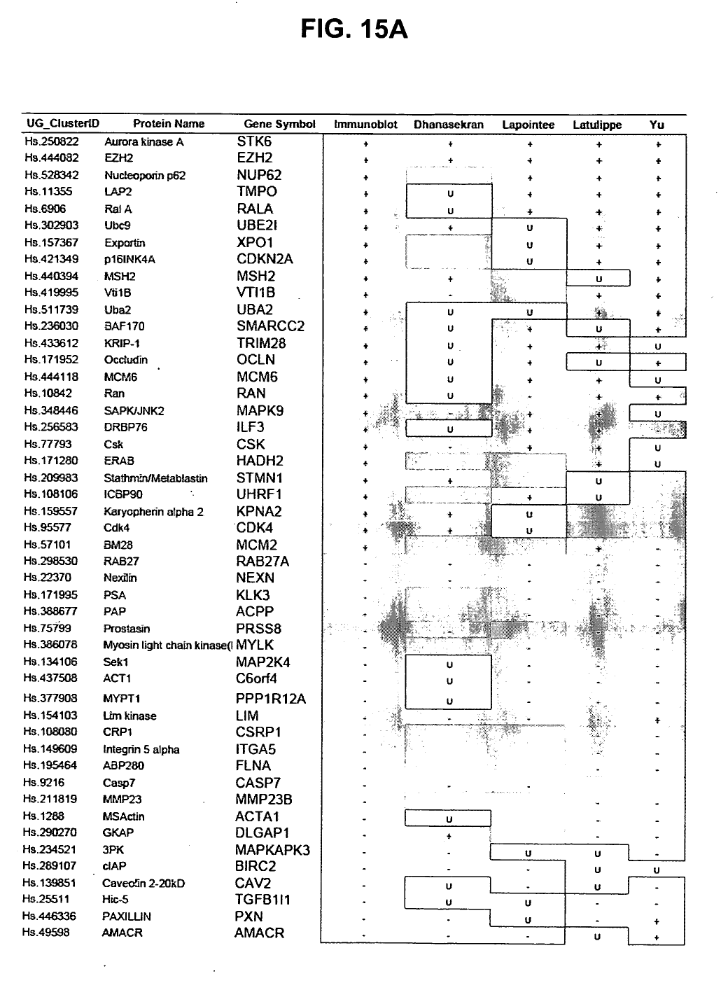

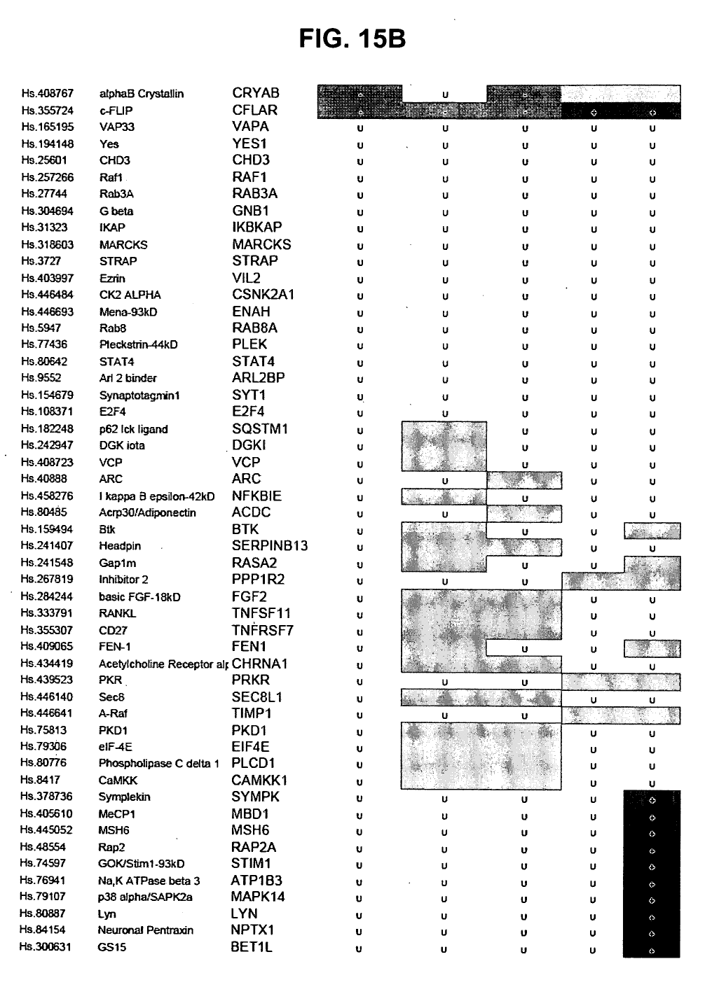

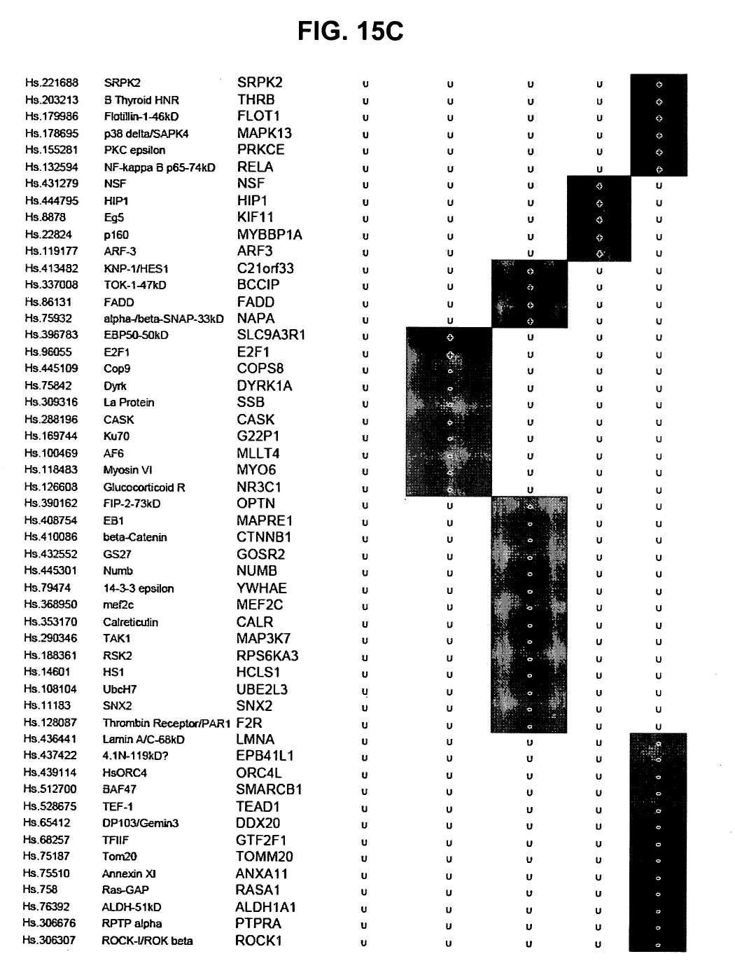

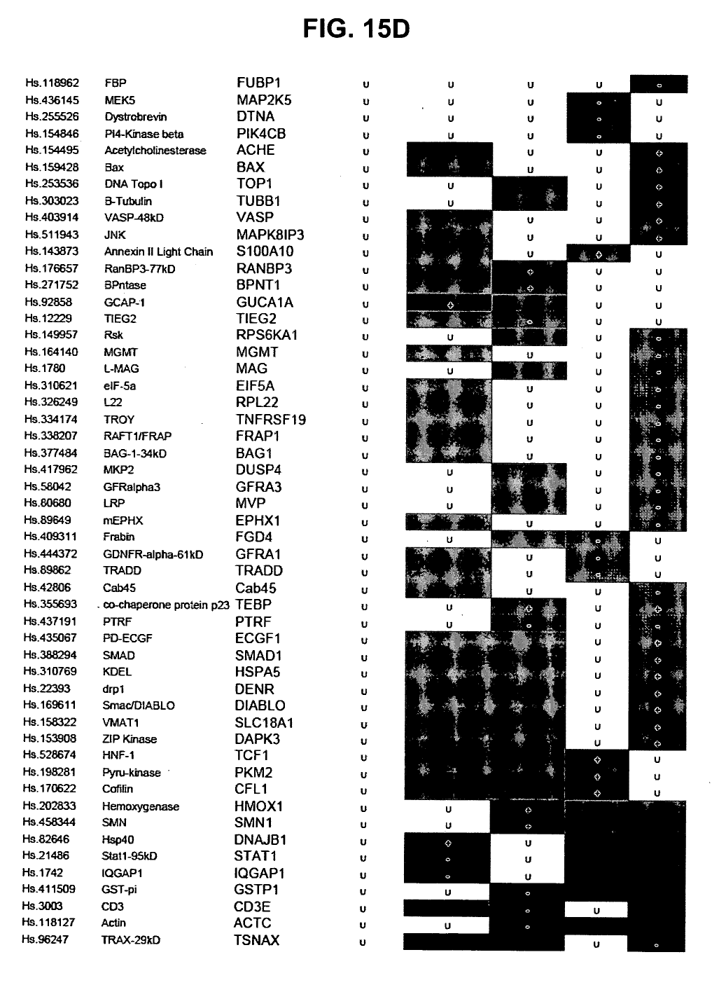

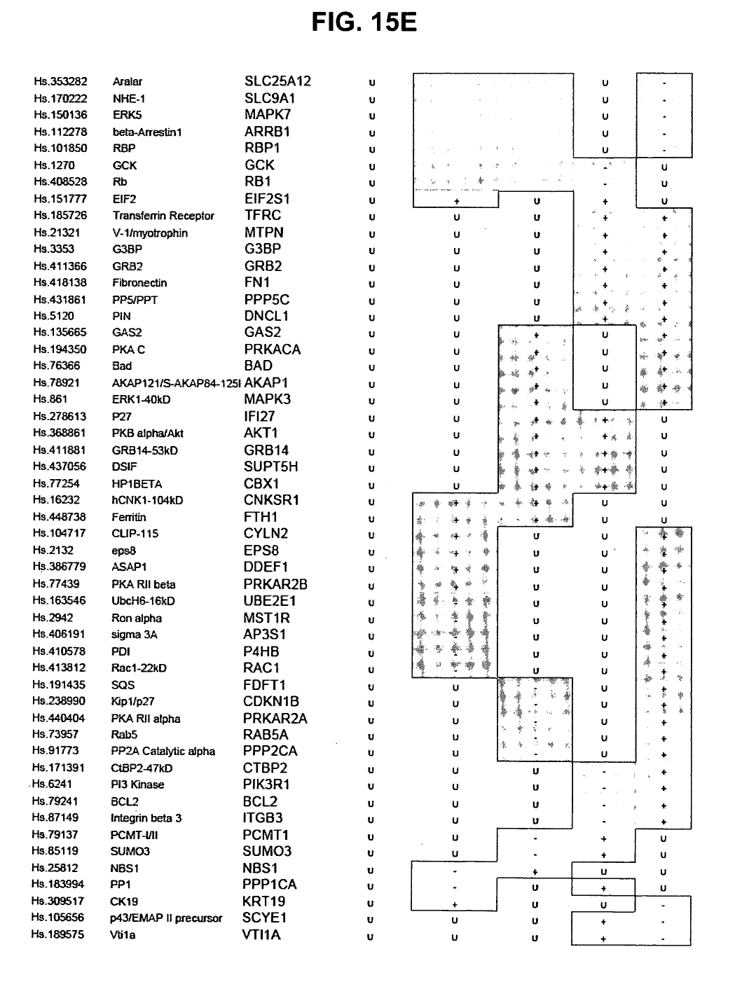

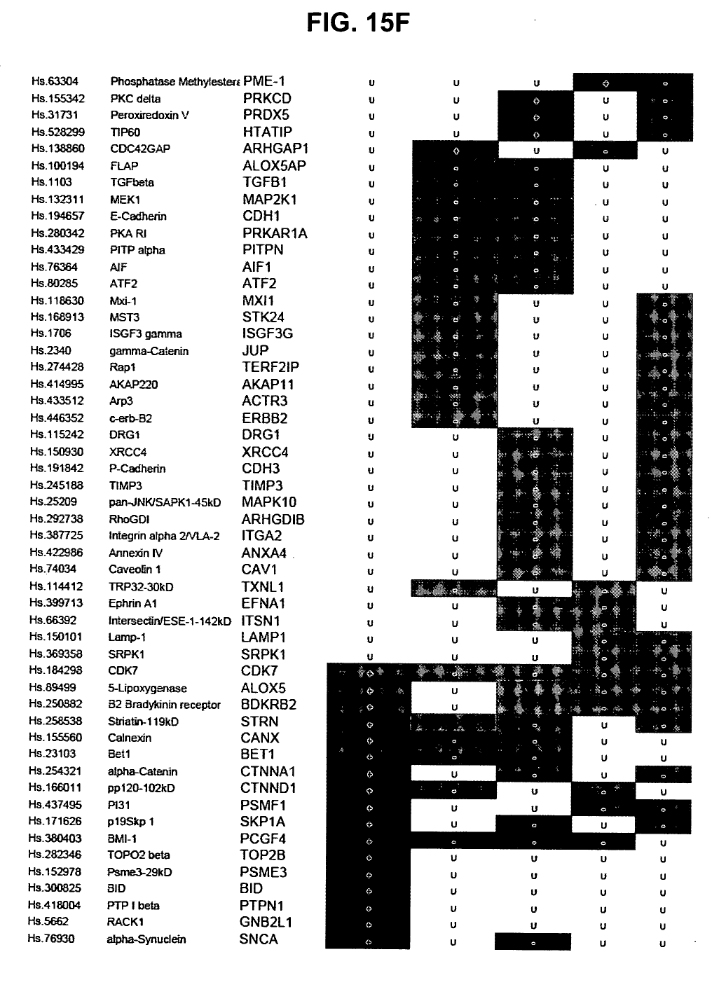

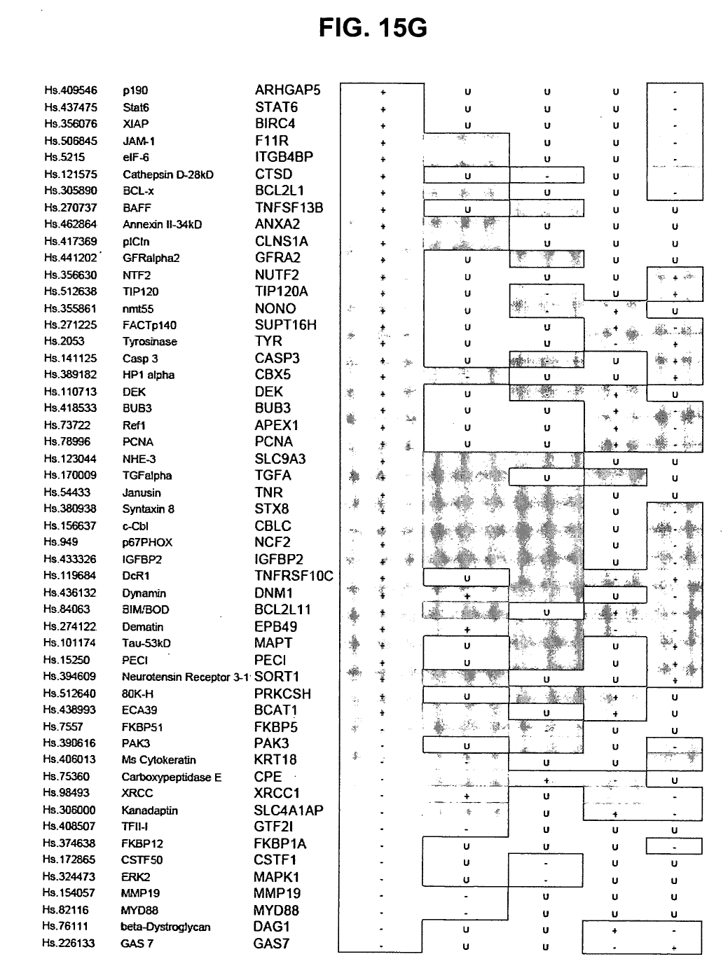

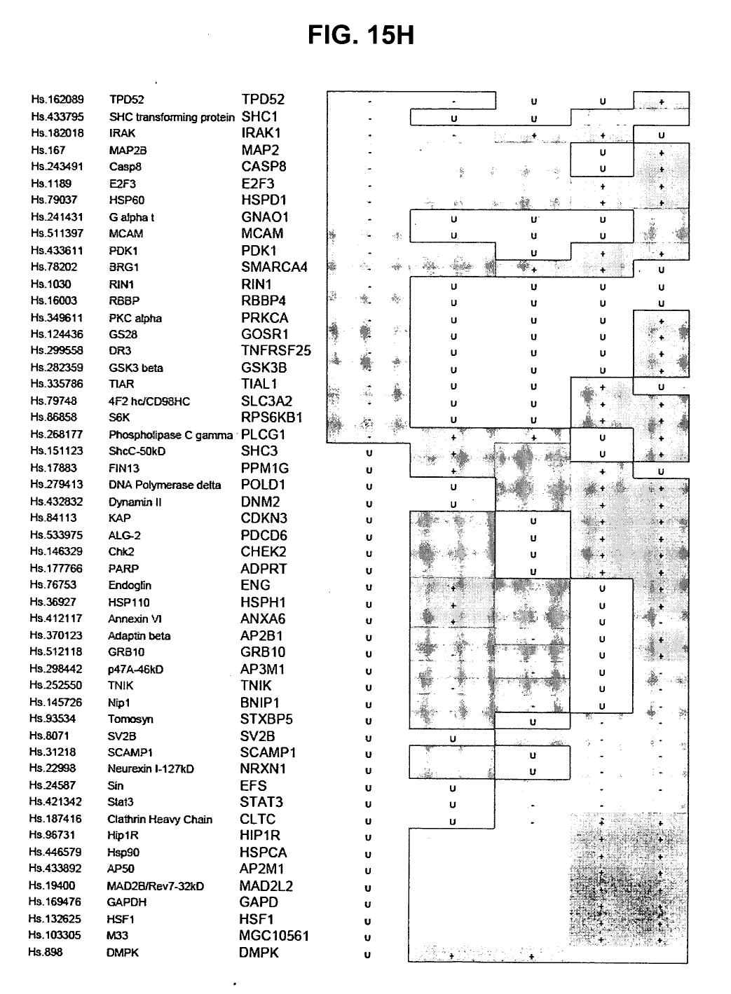

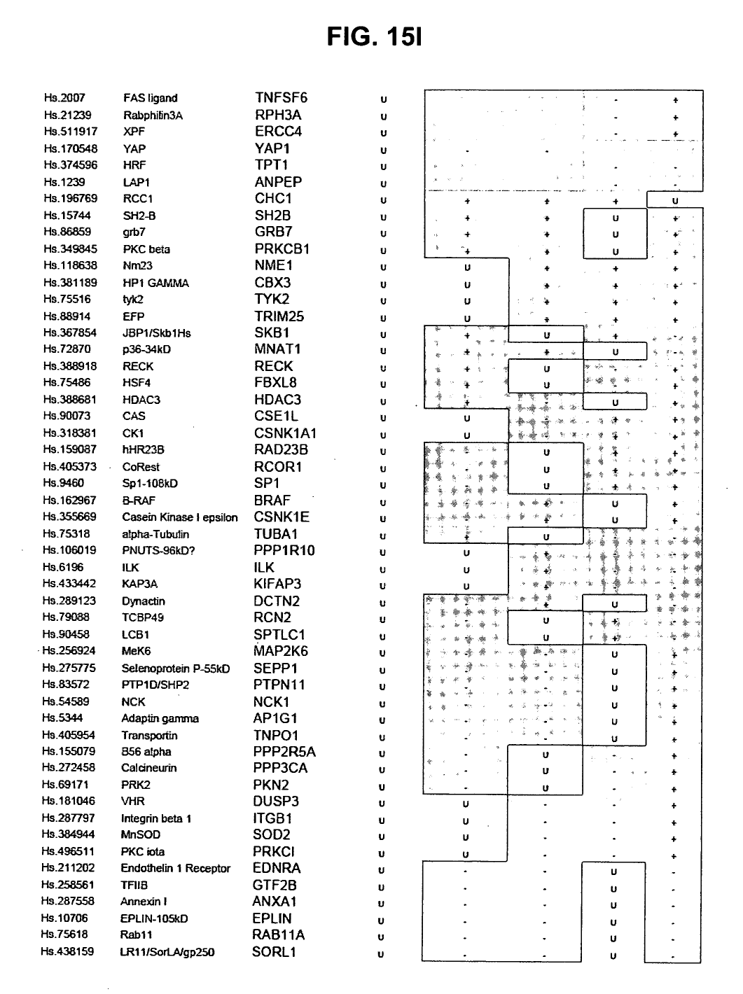

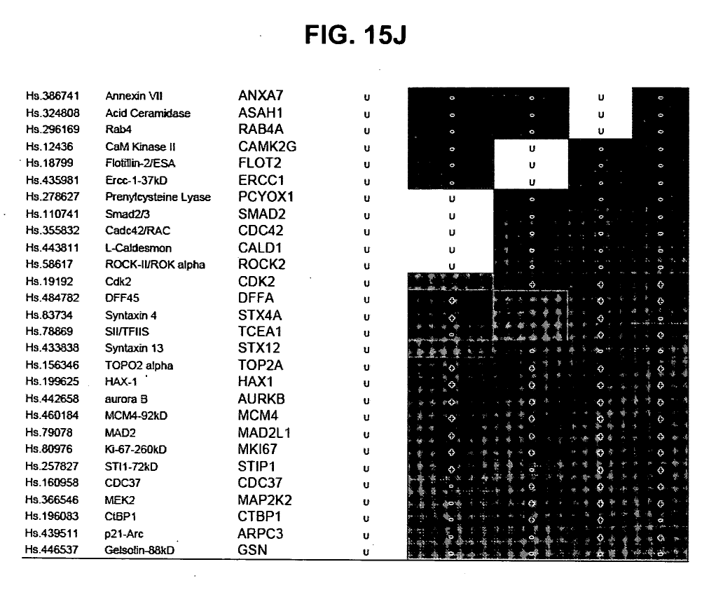

Table 10 (FIG. 15) shows proteomics alterations mapped to gene expression in metastatic prostate cancer relative to clinically localized prostate cancer.

TABLE 11
Concordant proteomic/genomic signature in metastatic prostate
cancer relative to clinically localized

| | | disease* | | | | |
|---|---|---|---|---|---|---|
| UG_ClusterID | NAME | Immunoblot | Dhanasekran et al. | Lapointee et al. | Latulippe et al. | Yu et al. |
| Hs.250822 | Aurora kinase A | + | + | + | + | + |
| Hs.444082 | EZH2 | + | + | + | + | + |
| Hs.528342 | Nucleoporin p62 | + | | + | + | + |
| Hs.11355 | LAP2 | + | U | + | + | + |
| Hs.6906 | Ral A | + | U | + | + | + |
| Hs.302903 | Ubc9 | + | + | U | + | + |
| Hs.157367 | Exportin | + | | U | + | + |
| Hs.421349 | p16INK4A | + | | U | + | + |
| Hs.440394 | MSH2 | + | + | | U | + |
| Hs.419995 | Vti1B | + | − | | + | + |
| Hs.511739 | Uba2 | + | U | U | + | + |
| Hs.236030 | BAF170 | + | U | + | U | + |
| Hs.433612 | KRIP-1 | + | U | + | + | U |
| Hs.171952 | Occludin | + | U | + | U | + |
| Hs.444118 | MCM6 | + | U | + | + | U |
| Hs.10842 | Ran | + | U | − | + | + |
| Hs.348446 | SAPK/JNK2 | + | − | + | + | U |
| Hs.256583 | DRBP76 | + | U | + | + | − |
| Hs.77793 | Csk | + | − | + | + | U |
| Hs.171280 | ERAB | + | | | + | U |
| Hs.209983 | Stathmin/Metablast | + | + | | U | |
| | in | | | | | |
| Hs.108106 | ICBP90 | + | | + | U | |
| Hs.159557 | Karyopherin alpha 2 | + | + | U | | |
| Hs.95577 | Cdk4 | + | + | U | | |
| Hs.57101 | BM28 | + | | | + | − |
| Hs.298530 | RAB27 | − | − | − | − | − |
| Hs.22370 | Nexilin | − | − | − | − | − |
| Hs.171995 | PSA | − | | − | − | − |
| Hs.388677 | PAP | − | | − | − | − |
| Hs.75799 | Prostasin | − | | − | − | − |
| Hs.386078 | Myosin light chain | − | − | | − | − |
| | kinase(MLCK) | | | | | |
| Hs.134106 | Sek1 | − | U | − | − | − |
| Hs.437508 | ACT1 | − | U | − | − | − |
| Hs.377908 | MYPT1 | − | U | − | − | − |
| Hs.154103 | Lim kinase | −− | − | − | − | + |
| Hs.108080 | CRP1 | − | | − | | − |
| Hs.149609 | Integrin 5 alpha | − | | − | | − |
| Hs.195464 | ABP280 | − | | − | | − |
| Hs.9216 | Casp7 | −− | − | | | |
| Hs.211819 | MMP23 | − | | − | | − |
| Hs.1288 | MSActin | −U | | − | | − |
| Hs.290270 | GKAP | −+ | | − | | − |
| Hs.234521 | 3PK | −− | U | U | | − |
| Hs.289107 | cIAP | −− | − | U | | U |
| Hs.139851 | Caveolin 2-20 kD | −U | − | U | | − |
| Hs.25511 | Hic-5 | −U | U | − | | − |
| Hs.446336 | PAXILLIN | −− | U | − | | + |
| Hs.49598 | AMACR | −− | − | U | | + |
| Hs.408767 | alphaB Crystallin | −U | − | | | |
| Hs.355724 | c-FLIP | −− | − | + | | + |

*“+”, “U”, or “−” denotes that the corresponding protein is increased, unchanged, or decreased respectively in metastatic prostate cancer relative to clinically localized prostate cancer.

### Example 4

### Breast Cancer Analysis

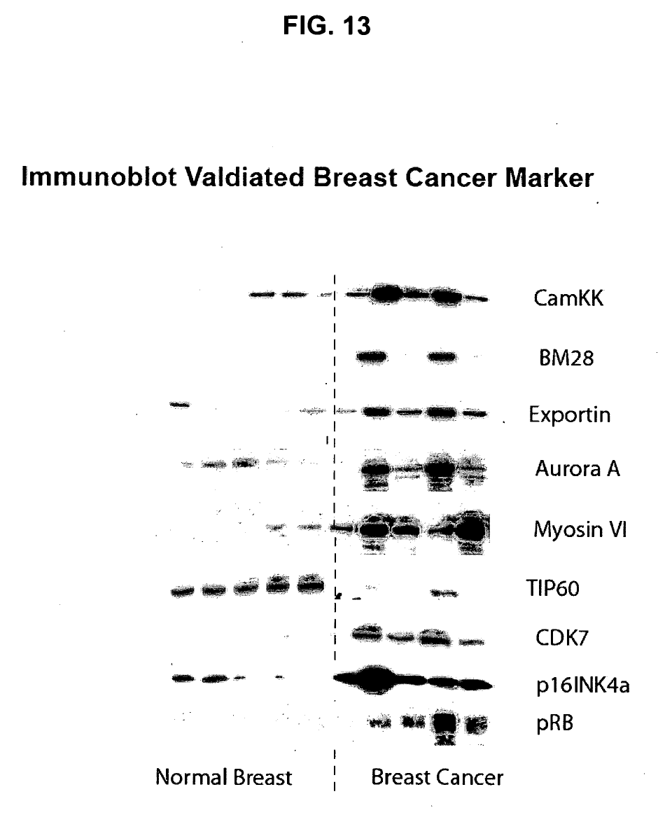

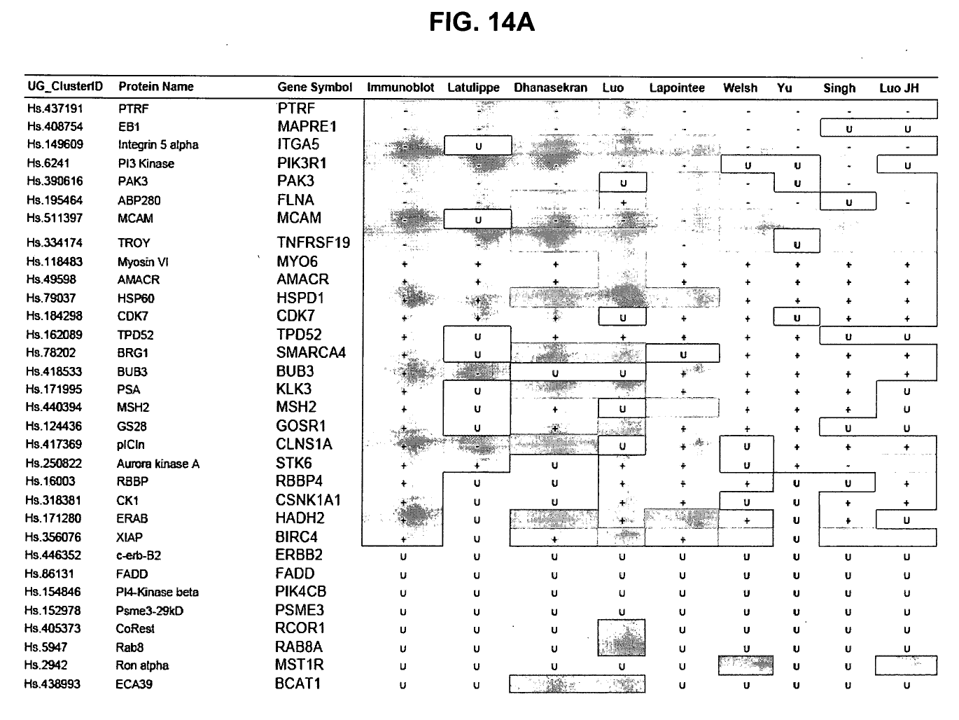

Further experiments were performed that analyzed expression profiles in breast cancer. Exemplary markers identified include CamKK, Myosin VI, Auroara A, exportin, BM28, CDK7, TIP60, and p16 INK 4a. Tissue microarray analysis is shown in FIG. 13.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

We claim:

1. A method for characterizing prostate tissue in a subject, comprising:
a) providing a prostate tissue sample from a subject; and
b) detecting the level of expression of a cancer marker selected from the group consisting of E2 ubiquitin ligase, UBc9, the cytosolic phosphoprotein stathmin, the death receptor DR3, and the Aurora A kinase (STK15), KRIP1 (KAP-1), Dynamin, CDK7, LAP2, Myosin VI, ICBP90, ELP/XIAP, CamKK, JAM1, PICIn, and p23 in said sample, thereby characterizing said prostate tissue sample.

2. The method of claim 1, wherein said detecting the level of expression of a cancer marker comprises detecting the presence of cancer marker mRNA.

3. The method of claim 2, wherein said detecting the level of expression of a cancer marker mRNA comprises exposing said cancer marker mRNA to a nucleic acid probe complementary to said cancer marker mRNA.

4. The method of claim 1, wherein said detecting the level of expression of a cancer marker comprises detecting the presence of a cancer marker polypeptide.

5. The method of claim 4, wherein said detecting the level of expression of a cancer marker polypeptide comprises exposing said cancer marker polypeptide to an antibody specific to said cancer marker polypeptide and detecting the binding of said antibody to said cancer marker polypeptide.

6. The method of claim 1, wherein said subject is a human subject.

7. The method of claim 1, wherein said sample comprises tumor tissue.

8. The method of claim 1, wherein said characterizing said prostate tissue comprises identifying a stage of prostate cancer in said prostate tissue.

9. The method of claim 8, wherein said stage of prostate cancer is selected from the group consisting of prostate carcinoma and metastatic prostate carcinoma.

10. The method of claim 1, further comprising the step of c) providing a prognosis to said subject.

11. The method of claim 10, wherein said prognosis comprises a risk of developing prostate cancer.

12. A kit for characterizing prostate tissue in a subject, comprising:
a) a reagent sufficient for the detection of the level of expression of two or more cancer markers selected from the group consisting of a cancer marker selected from the group consisting of E2 ubiquitin ligase, UBc9, the cytosolic phosphoprotein stathmin, the death receptor DR3, and the Aurora A kinase (STK15), KRIP1 (KAP-1), Dynamin, CDK7, LAP2, Myosin VI, ICBP90, ILP/XIAP, CamKK, JAM 1, PICIn, and p23.

13. The kit of claim 12, wherein said two or more comprises three or more.

14. The kit of claim 12, wherein said two or more comprises five or more.

15. The kit of claim 12, wherein said reagent comprises a nucleic acid probe complementary to said cancer marker mRNA.

16. The kit of claim 12, wherein said reagent comprises an antibody that specifically binds to said cancer marker polypeptide.

17. A method for characterizing breast tissue in a subject, comprising:
a) providing a breast tissue sample from a subject; and
b) detecting the level of expression of a cancer maker selected from the group consisting of CamKK, Myosin VI, Auroara A, exportin, BM28, CDK7, TIP60, and p16 INK 4a in said sample, thereby characterizing said breast tissue sample.

18. The method of claim 17, wherein said detecting the level of expression of a cancer marker comprises detecting the presence of cancer marker mRNA.

19. The method of claim 17, wherein said detecting the level of expression of a cancer marker comprises detecting the presence of a cancer marker polypeptide.

20. The method of claim 18, further comprising the step of c) providing a prognosis to said subject.
